(19) United States
(12) Patent Application Publication
(10) Pub. No.: US 2018/0326102 A1
(43) Pub. Date: Nov. 15, 2018

(54) SYSTEMS, METHODS, AND COMPOSITIONS FOR IMAGING ANDROGEN RECEPTOR AXIS ACTIVITY IN CARCINOMA, AND RELATED THERAPEUTIC COMPOSITIONS AND METHODS

(71) Applicant: Memorial Sloan Kettering Cancer Center, New York, NY (US)

(72) Inventor: David Ulmert, New York, NY (US)

(21) Appl. No.: 15/777,319

(22) PCT Filed: Nov. 18, 2016
(86) PCT No.: PCT/US16/62818

§ 371 Date: May 18, 2018

Related U.S. Application Data
(60) Provisional application No. 62/257,179, filed on Nov. 18, 2015.

(51) Int. Cl. A61K 51/10; A61K 49/00; A61K 31/568; A61K 31/57

(52) U.S. Cl. CPC A61K 51/1051; A61K 51/1072; A61K 31/57; A61K 31/568; A61K 49/0058

(57) ABSTRACT

Presented herein are systems, compositions, and methods for immuno-PET/SPECT and/or immuno-fluorescence-guided imaging of tissue for diagnosing, localizing, radiation dose planning, and/or evaluating therapy response (e.g., anti-androgen receptor therapeutics, surgery and external irradiation) in cancer (e.g., androgen receptor (AR) positive breast cancer). In other embodiments, for example, the invention is directed to radio-immunotherapy (RIT) treatment of AR-positive breast cancer by administration (e.g., injection) of a free-PSA and/or free hK2 antibody labelled with a radioisotope after KLK2 and KLK3 induction by progesterone, testosterone, and/or irradiation.

### CROSS-REFERENCE TO RELATED APPLICATION

This application claims the benefit of U.S. Provisional Application No. 62/257,179, filed Nov. 18, 2015, the contents of which is hereby incorporated by reference herein in its entirety.

### GOVERNMENT SUPPORT

This invention was made with government support under grant numbers CA096945, CA127768, CA092629, and CA008748 awarded by the National Institutes of Health. The government has certain rights in the invention.

### TECHNICAL FIELD

Presented herein are systems, methods, and compositions for imaging, diagnosing, and/or treating cancer, for example, androgen receptor positive breast cancer.

### BACKGROUND

Activation of the androgen receptor (AR) signaling axis contributes to prostate cancer (PCa) progression throughout the entire course of the disease, including the castration resistant state. Following initial response to inhibition with androgen deprivation treatment, a mainstay of PCa treatment, AR-pathway reactivation inevitably occurs. Reactivation has been attributed to gene amplification, intratumoral androgen synthesis, constitutively active AR variants, and other mechanisms. AR is also differentially expressed in several breast cancer (BCa) subtypes, though without a clearly defined role. This includes aggressive triple negative BCa (TN-BCa), where AR expression is correlated with decreased survival. Recent trials have focused on AR inhibition as an approach to stabilize this otherwise unmanageable disease. Thus, quantifying lesion-specific AR pathway activity represents a critical unmet need that assists in treatment selection, as a pharmacodynamic marker of pathway inhibition, and represents a non-invasive biomarker of therapeutic efficacy.

Prostate specific antigen (PSA), also known as kallikrein-3 (KLK3), is a commonly used biomarker of prostate cancer. Although PSA is androgen regulated, concentrations in the blood are a function of degree of tumor differentiation, physiological factors and total tumor burden, making PSA unsuitable as a measure of pathway activation.

In humans, KLK2 is a gene that encodes kallikrein related peptidase 2 (hK2), a trypsin-like enzyme with AR-driven expression specific to prostate tissue, PCa and AR-positive BCa tissues. hK2 is activated by Transmembrane Protease, Serine 2 (TMPRSS2) and secreted into the ducts of the prostate, where it initiates a cascade that cleaves semenogelin, the extracellular matrix in ejaculate, to enhance sperm motility. hK2 in man is exclusively expressed in prostatic tissues (FIG. 9D). Similar to PSA, retrograde release of catalytically inactive hK2 into the blood occurs when the highly structured organization of the prostate is compromised upon hypertrophy or malignant transformation.

The AR axis is active in many difficult-to-treat breast cancer (BCa) subtypes, such as triple negative breast cancer (TN-BCa), and in treatment-resistant disease (e.g., anti-estrogen therapy or tamoxifen resistance). KLK2 expression is low in BCa cells, but can be increased by treatment with progesterone, testosterone, and/or external irradiation.

Inhibition of disease processes by drug binding to secreted antigens is established in clinical practice. Targets of biologics include vascular endothelial growth factor, receptor activator of nuclear factor kappa-B and tissue necrosis factor, among others. Imaging agents or drug conjugates directed to secreted antigens have been far less successful, as antibody-bound complexes wash out of the disease site. This has limited targets for PCa to cell surface receptors, which usually have poor tissue- or disease-restricted expression (FIGS. 9A-9C), as indicated from the integrated in silico transcriptomics database (IST, Medisapiens).

Direct imaging of AR abundance and measurement of receptor occupancy has previously been achieved using ¹⁸F-FDHT (a radiolabeled analog of the androgen testosterone). However, uptake of this agent does not correlate with PSA decline or response, both of which are positively tied to AR pathway activity and not simply the amount of receptor. In the VCaP prostate cancer model, the rapid metabolism and abdominal clearance of this agent (FIG. 28) results in limited contrast of tumor to background structures.

Therefore, there is a need for improved systems, methods, and compositions to characterize disease, guide therapy, and monitor response of treatment.

### SUMMARY

Presented herein are systems, compositions, and methods involving the use of murine and/or humanized antibodies targeting free PSA (such as 5A10) and/or free hK2 (such as 11B6) for in vivo targeting of androgen receptor (AR) positive cancer (e.g., breast cancer, e.g., prostate cancer). For example, the antibodies can be used alone (e.g., 5A10 or 11B6) or in combination (e.g., 5A10 and 11B6).

For example, in certain embodiments, the present disclosure is directed to immuno-PET/SPECT and/or immuno-fluoresce-guided imaging for diagnosing, localizing, radiation dose planning, and/or evaluating therapy response (e.g., anti-androgen receptor therapeutics, surgery and external irradiation) in androgen receptor (AR) positive breast cancer or PCa. Evaluation can include monitoring of AR-upregulation of KLK2 and KLK3 in response to external irradiation.

In other embodiments, for example, the present disclosure is directed to radio-immunotherapy (RIT) treatment of AR-positive breast cancer by administration (e.g., injection) of a free-PSA and/or free hK2 antibody labelled with a radioisotope after KLK2 and KLK3 induction by progesterone, testosterone or irradiation.

In other embodiments, for example, the present disclosure provides an antibody-based platform directed to a secreted antigen that uses Fc-receptor mediated internalization for cancer imaging and therapy.

In one aspect, the invention is directed to a method of assessing androgen receptor activity in a subject, the method comprising: administering, to the subject, a tracer-labelled hK2-specific or PSA-specific antibody; and detecting the presence of the labeled antibody in a tissue of the subject.

In certain embodiments, the tissue comprises breast tissue.

In certain embodiments, the antibody comprises a murine or humanized antibody. In certain embodiments, the antibody comprises murine or humanized 11B6, and/or murine or humanized 5A10.

In certain embodiments, the tracer comprises a radionuclide. In certain embodiments, the radionuclide is a member selected from the group consisting of ¹¹C, ⁶⁴Cu, ¹²⁴I, ¹¹¹In, ¹⁷⁷Lu, ¹⁵O, ¹⁸F, ⁶⁸Ga, ⁸⁹Zr, and ⁸²Rb.

In certain embodiments, the method comprises administering hu11B6 labeled with ⁸⁹Zr or administering ⁸⁹Zr-DFO-hu11B6.

In certain embodiments, the detecting is performed via PET imaging, CT imaging, SPECT imaging, and/or in vivo imaging. In certain embodiments, the method comprises detecting the presence and/or activity of the androgen receptor (AR) axis. In certain embodiments, the method comprises detecting the presence of the labeled antibody in the tissue at a time frame selected from the group consisting of at least 24 hours after administration of the labeled antibody to the subject, at least 48 hours after administration of the labeled antibody to the subject, at least 100 hours after administration of the labeled antibody to the subject, and at least 120 hours after administration of the labeled antibody to the subject.

In certain embodiments, the labeled antibody accumulates and internalizes in tumor cells, thereby allowing visualization/tracking over long periods of time.

In certain embodiments, the tissue has metastasized to bone.

In certain embodiments, the method comprises detecting the presence of the labeled antibody in the tissue over a period of multiple time intervals. In certain embodiments, the detecting is for real-time monitoring/visualization.

In certain embodiments, the method comprises detecting the presence of the labeled antibody in the tissue at multiple times, including at least one detection after a time selected from the group consisting of at least 24 hours following administration of the labeled antibody, after at least 48 hours following administration of the labeled antibody, after at least 100 hours following administration of the labeled antibody, and after at least 120 hours following administration of the labeled antibody.

In certain embodiments, the method further comprises one or more of (i) to (vi), as follows: (i) identifying the presence of cancer in the subject; (ii) localizing a cancer in the subject; (iii) quantitatively assessing androgen receptor pathway activity in the subject/cancer; (iv) planning radiation dose(s) in a course of treatment of the subject; (v) determining one or more pharmacodynamics parameters for the subject; and (vi) evaluating treatment efficacy. In certain embodiments, the cancer comprises a member selected from the group consisting of breast cancer (BCa), AR-positive breast cancer, triple negative breast cancer (TN-BCa), and any metastasis of BCa. Ar-positive breast cancer, and TN-BCa.

In certain embodiments, the determining of one or more pharmacodynamics parameters for the subject is in conjunction with treatment of the subject with one or more drugs.

In certain embodiments, the evaluating comprises evaluating therapy response.

In certain embodiments, the method comprises monitoring AR-upregulation of KLK2 and/or KLK3. In certain embodiments, the AR-upregulation of KLK2 and/or KLK3 is in response to external irradiation.

In another aspect, the invention is directed to a method of assessing androgen receptor activity in a subject, the method comprising: administering, to the subject, a tracer-labelled hK2-specific or PSA-specific antibody; and detecting the presence of the labeled 11B6 in a tissue of the subject.

In certain embodiments, the tissue comprises breast tissue.

In certain embodiments, the tracer-labelled hK2-specific or PSA-specific antibody comprises a murine or humanized antibody. In certain embodiments, the murine or humanized antibody comprises a murine or humanized 11B6 (hu11B6), and/or murine or humanized 5A10 (hu5A10).

In certain embodiments, the tracer comprises a fluorophore.

In certain embodiments, the method comprises administering hu11B6 labeled with a tag comprising a member selected from the group consisting of a near infrared fluorophore and a Cy5.5.

In certain embodiments, the detecting is performed via fluorescent imaging or in vivo imaging. In certain embodiments, the method comprises detecting the presence and/or activity of the androgen receptor (AR) axis.

In certain embodiments, the method further comprises one or more of (i) to (vi), as follows: (i) identifying the presence of cancer in the subject; (ii) localizing the cancer in the subject; (iii) quantitatively assessing androgen receptor pathway activity in the subject/cancer; (iv) planning radiation dose(s) in a course of treatment of the subject; (v) determining one or more pharmacodynamics parameters for the subject; and (vi) evaluating treatment efficacy.

In certain embodiments, the cancer comprises a member selected from the group consisting of breast cancer (BCa), AR-positive breast cancer, triple negative breast cancer (TN-BCa), and any metastasis of BCa, AR-positive breast cancer, and TN-BCa.

In certain embodiments, the determining of one or more pharmacodynamics parameters for the subject is determined in conjunction with treatment of the subject with one or more drugs.

In certain embodiments, the method comprises monitoring AR-upregulation of KLK2 and/or KLK3. In certain embodiments, the AR-upregulation of KLK2 and/or KLK3 is in response to external irradiation.

In another aspect, the invention is directed to a method of treating AR-positive breast cancer with one or more agents/treatments selected from the group consisting of: (i) a radionuclide-labelled hK2-specific or PSA-specific antibody; and (ii) at least one member selected from the group consisting of progesterone, testosterone, and external irradiation, which method comprises administering the one or more agents/treatments to a subject suffering from or susceptible to AR-positive breast cancer, so that the subject is receiving therapy with a combination of (i) and (ii) above.

In certain embodiments, the radionuclide comprises a member selected from the group consisting of ⁹⁰Y, ¹³¹I, ²¹¹At, ¹¹¹In, ¹⁷⁷Lu, ²²⁷Th, ¹⁴⁹Tb, ²¹²Bi, ²¹³Bi, ²²⁵Ac, ⁸²Rb, and ²²³Ra. In certain embodiments, the radionuclide-labelled hK2-specific or PSA-specific antibody comprises a member selected from the group consisting of a humanized 11B6 (hu11B6), humanized 5A10 (hu5A10), hu11B6 labeled with an alpha-particle-emitting radionuclide, hu11B6 labeled with ²²⁵Ac, and ²²⁵Ac-DOTA-hu11B6.

In another aspect, the invention is directed to a method of treating AR-positive breast cancer or any metastasis of AR-positive breast cancer, the method comprising administering, to a subject suffering from or susceptible to the disease or condition, a radionuclide-labelled hK2-specific or PSA-specific antibody.

In certain embodiments, the radionuclide comprises a member selected from the group consisting of ⁹⁰Y, ¹³¹I, ²¹¹At, ¹⁴⁹Tb, ²¹²Bi, ²¹³Bi, ²²⁵Ac, ¹¹¹In, ¹⁷⁷Lu, ²²⁷Th, and ²²³Ra.

In another aspect, the invention is directed to a composition comprising one or more agents selected from the group consisting of: (i) a radionuclide-labelled hK2-specific or PSA-specific antibody; and (ii) at least one member selected from the group consisting of progesterone, testosterone, and external irradiation, for use in a method of treating AR-positive breast cancer in a subject suffering from or susceptible to AR-positive breast cancer, wherein the treating comprises: delivering a combination of (i) and (ii) above to the subject.

In another aspect, the invention is directed to a composition comprising one or more agents selected from the group consisting of: (i) a radionuclide-labelled hK2-specific or PSA-specific antibody; and (ii) at least one member selected from the group consisting of progesterone, testosterone, and external irradiation, for use in therapy.

In another aspect, the invention is directed to a composition comprising one or more agents selected from the group consisting of: (i) a radionuclide-labelled hK2-specific or PSA-specific antibody; and (ii) at least one member selected from the group consisting of progesterone, testosterone, and external irradiation, for use in a method of in vivo diagnosis of AR-positive breast cancer in a subject in a subject suffering from or susceptible to AR-positive breast cancer, wherein the in vivo diagnosis comprises: delivering a combination of (i) and (ii) above to the subject.

In another aspect, the invention is directed to a composition comprising one or more agents selected from the group consisting of: (i) a radionuclide-labelled hK2-specific or PSA-specific antibody; and (ii) at least one member selected from the group consisting of progesterone, testosterone, and external irradiation, for use in in vivo diagnosis.

In another aspect, the invention is directed to a composition comprising one or more agents selected from the group consisting of: (i) a radionuclide-labelled hK2-specific or PSA-specific antibody; and (ii) at least one member selected from the group consisting of progesterone, testosterone, and external irradiation, for use in (a) a method of treating AR-positive breast cancer in a subject or (b) a method of in vivo diagnosis of AR-positive breast cancer in a subject, wherein the method comprises: delivering a combination of (i) and (ii) above to the subject.

In certain embodiments, the radionuclide comprises a member selected from the group consisting of ⁹⁰Y, ¹³¹I, ²¹¹At, ¹¹¹In, ¹⁷⁷Lu, ²²⁷Th, ¹⁴⁹Tb, ²¹²Bi, ²¹³Bi, ²²⁵Ac, ⁸²Rb, and ²²³Ra. In certain embodiments, the radionuclide-labelled hK2-specific or PSA-specific antibody comprises a member selected from the group consisting of a humanized 11B6 (hu11B6), humanized 5A10 (hu5A10), hu11B6 labeled with an alpha-particle-emitting radionuclide, hu11B6 labeled with ²²⁵Ac, and ²²⁵Ac-DOTA-hu11B6.

In another aspect, the invention is directed to a composition comprising a radionuclide-labelled hK2-specific of PSA-specific antibody for use in a method of treating AR-positive breast cancer or any metastasis of AR-positive breast cancer in a subject suffering from or susceptible to the disease or condition, wherein the treating comprises delivering the composition to the subject.

In another aspect, the invention is directed to a composition comprising a radionuclide-labelled hK2-specific of PSA-specific antibody for use in therapy.

In another aspect, the invention is directed to a composition comprising a radionuclide-labelled hK2-specific of PSA-specific antibody for use in a method of in vivo diagnosis of AR-positive breast cancer or any metastasis of AR-positive breast cancer in a subject suffering from or susceptible to the disease or condition, wherein the in vivo diagnosis comprises delivering the composition to the subject.

In another aspect, the invention is directed to a composition comprising a radionuclide-labelled hK2-specific of PSA-specific antibody for use in in vivo diagnosis.

In another aspect, the invention is directed to a composition comprising a radionuclide-labelled hK2-specific of PSA-specific antibody for use in (a) a method of treating AR-positive breast cancer or any metastasis of AR-positive breast cancer in a subject or (b) a method of in vivo diagnosis of AR-positive breast cancer or any metastasis of AR-positive breast cancer in a subject, wherein the method comprises delivering the composition to the subject.

In certain embodiments, the radionuclide comprises a member selected from the group consisting of ⁹⁰Y, ¹³¹I, ²¹¹At, ¹⁴⁹Tb, ²¹²Bi, ²¹³Bi, ²²⁵Ac, ¹¹¹In, ¹⁷⁷Lu, ²²³Ra, and ²²⁷Th.

The description of elements of one aspect of the invention (e.g., features of a system, method, or composition) can be applied as elements of other aspects of the invention (e.g., features of a system, method, and/or composition) as well.

### BRIEF DESCRIPTION OF THE DRAWINGS

FIGS. 1A-1D show prostate cancer targeting and accumulation of Active-hK2 Targeted Radiolabeled Antibody.

FIG. 1A shows coronal slices through xenograft (LNCaP) bearing mice, over time. The long-lived PET isotope ⁸⁹Zr enables longitudinal imaging, which shows continued uptake over 10 d. Schematic of tumor location Tumor (T) on flank, and Liver (L). B

FIGS. 1B and 1C show biodistribution of mass escalation study at 320 h, with time time activity curves in % IA/g of tumor (squares) and blood (circles) for 50, 150 and 300 μg doses (top to bottom of FIG. 1C).

FIG. 1D shows greater uptake in the higher-hK2 producing VCaP in comparison to the LNCaP and non-producing DU145 xenografts indicates specificity, which can also be blocked with cold antibody (1 mg).

FIGS. 2A-2C show that ⁸⁹Zr-DFO-11B6 delineates osteolytic and osteoblastic bone metastases. The radiotracer is able to distinguish both LNCaP osteolytic (FIG. 1A), VCaP osteoblastic tumors (FIG. 1B), and PC3 AR- and hK2-negative osteolytic lesions (FIG. 1C) in the mouse tibia. X-ray computed tomography of the electron dense bone (left-most column; CT) shows the loss of bone in the LNCaP and PC3 models. The intensity of signal again recapitulates the relative expression levels of the two AR-positive cell lines (PET column). 3-dimensional PET/CT fusion images with opaque bone (second from right) and transparent bone (right-most column) show that these metastases are restricted from the surrounding. Low-levels of nonspecific ⁸⁹Zr uptake at the epiphyseal growth plate is seen in all models. Quantitation of uptake and kinetics are shown in FIGS. 13A-13B.

FIGS. 3A-3G show intracellular accumulation of 11B6-hK2.

FIGS. 3A-3E show that the whole prostate and seminal vesicles (prostate package) were removed from Pb_KLK2 mice 72 h after injection of Cy5.5-11B6 and ⁸⁹Zr-DFO-11B6 for whole mount fluorescence (FIG. 3A), confocal microscopy (FIG. 3B), and autoradiography (FIG. 3C). Intense uptake in the glandular structures of the ventral prostate (arrow), with lower uptake in the dorsolateral prostate (*). No uptake in non-transgenic mice was observed (not shown). Radio- and fluorescent signal correlated with the ventral prostate gland by H&E (FIG. 3D), which is confirmed by Androgen Receptor (AR) staining that is intense in the ventral prostate (scale is 500 μm) (FIG. 3E).

FIGS. 3F-3G show that following incubation with LNCaP prostate cancer cells, the 11B6 antibody co-localizes with FcRN early (FIG. 3F) and is then trafficked to acidified lysosomes as indicated by increased fluorescence from pH-responsive dye labeled 11B6 (pH-11B6) (FIG. 3G).

FIG. 4 shows noninvasive annotation of prostate cancer development by ⁸⁹Zr-11B6. Representative pelvic fused ⁸⁹Zr-11B6 (50 μg) PET/CT acquisitions of cancer susceptible hK2-expressing mice (Pb_KLK2×Hi-Myc mice) throughout development of adenocarcinoma. The age in weeks is displayed with insert.

FIGS. 5A-5F show lesion response to treatment.

FIG. 5A shows representative PET imaging with ⁸⁹Zr-11B6 on an intra-osseous LNCaP-AR model before (left) and after (right) castration.

FIGS. 5B-5C show that quantification of imaging results of ⁸⁹Zr-11B6 radiotracer uptake reflects AR-driven luciferase signal changes in the LNCaP-AR cell line.

FIG. 5D shows, in contrast to FIG. 5C, that PSA blood concentration values remained unchanged.

FIG. 5E shows that conventional ¹⁸F—NaF imaging was also conducted before (left) and after castration (right) prior to ⁸⁹Zr-11B6.

FIG. 5F shows that quantitation of bone scan uptake values illustrates continued bone turnover at the site of the resolved lesion. Imaging experiments (n=5) and PSA assay (n=4), per group (Table 6).

FIGS. 6A-6E show characterization of drug response to surgical castration and adjuvant androgen receptor blockage. Noninvasive longitudinal quantification of castration and anti-androgen therapy with ⁸⁹Zr-11B6. Pb_KLK2×Hi-Myc mice were imaged before treatment, after castration (6 weeks post-surgery) and after adjuvant therapeutic intervention (4 weeks after either vehicle or enzalutamide (ENZ). Two representative subjects in both the vehicle (PBS) (FIG. 6A) and Enzalutamide treatment-groups (FIG. 6B).

FIG. 6C shows quantification (mean % ID/g) enabled by ⁸⁹Zr-11B6 of uptake of all mice pre- and post-castration.

FIGS. 6D and 6E show the mean uptake in the Vehicle (n=6) (FIG. 6D) and Enzalutamide groups (n=4) (FIG. 6E) through the entire adjuvant treatment regimen. Reactivation in the castration plus androgen receptor blockade group was not significant (n.s.).

FIGS. 7A and 7B show AR-increase after irradiation of two AR-positive BCa cell lines (BT474 and MFM223).

FIG. 8 shows a survival graph after injecting ²²⁵Ac-DOTA-hu11B6 in DHT-stimulated (e.g., expression of KLK2) and in non-DHT stimulated mice (e.g., non-KLK2 expression).

FIGS. 9A-9D show anatomical and disease-specific gene expression of candidate targets. Targeted agents for disease identification, characterization, and therapy include FIG. 9A) six transmembrane epithelial antigen of the prostate 1 (STEAP1), (FIG. 9B) prostate-specific membrane antigen (FOLH1), and (FIG. 9C) GCPR bombesin receptor (BSR3). FIG. 9D shows AR-activity regulated human kallikrein-related peptidase 2 (KLK2) is restricted to the prostate and prostate-derived tissue, as well as adenocarcinoma of the breast under sex-steroid stimulation. Median expression is shown as a horizontal line, with 25 and 75 percentiles as lower and upper bounds of the boxes, with whiskers and outlier points extending to cover the remaining data. Data from the In Silico Transcriptomics Online database, an integrated human gene expression catalog of 60 healthy tissues (light speckling), 104 malignant, and 64 other disease types (dark speckling).

FIG. 10 shows a competition assay comparing the affinity of non-labeled 11B6 (square) to DFO-conjugated (open circle), as well as ⁸⁹Zr-labeled DFO-11B6 (closed circle). No significant differences in capture efficacy of free hK2 are noted for the conjugated or radiolabeled constructs.

FIGS. 11A and 11B show ⁸⁹Zr-DFO-11B6 uptake and hK2 expression.

FIG. 11A shows protein expression and uptake of the tracer were correlated. Percent injected activity values were assessed by gamma-counting, and hK2 from lysate was measured by time-resolved immunofluorometric assay. hK2 protein values are expressed as ng per mg of total protein.

FIG. 11B shows that implanted 22Rv1 xenografts into the flank of castrated Balb/c nu/nu mice was used to evaluate the uptake of the tracer in a model of patients who have failed hormone therapy. Biodistribution demonstrates uptake at the tumor, through continued AR-driven hK2 expression.

FIGS. 12A-12D show relative expression of putative prostate markers in prostate cancer cell lines. RT-PCR was performed on 7 commonly used prostate cancer cell lines for genes of interest which included (FIG. 12A) KLK2 (encoding hK2), (FIG. 12B) FOLH1 (encoding PSMA, prostate-specific membrane antigen), and (FIG. 12C) KLK3 (encoding PSA). FIG. 12D shows neonatal Fc Receptor Gene Expression encoding the IgG-binding neonatal Fc receptor, across a panel of prostate cancer cell lines.

FIGS. 13A and 13B show time-activity curves of LNCaP-AR subcutaneous and orthotopic xenografts. FIG. 13A shows the kinetics of uptake measured as % IA/g in the flank xenograft model are faster than in (FIG. 13B) an orthotopic bone model. Time-activity curves were plotted noninvasively from dynamic PET acquisitions at the times indicated and show tumor (square) and blood (circle) values. Blood values were assessed from the mean % IA/g of volumes of interest defined around the heart from PET datasets.

FIGS. 14A-14C show ⁸⁹Zr-DFO-11B6 prostate and hK2-specific imaging in transgenic healthy and diseased mice. Sagittal and oblique views of three-dimensional ⁸⁹Zr-11B6 (50 μg) PET/CT fusion volumes of the pelvis in representative mice, with surface-rendered skeleton, 96 h after administration.

FIG. 14A shows no uptake of the radiotracer is seen in a wild-type C57Bl/6 mouse (42 weeks).

FIG. 14B shows a representative image of a mouse (51 weeks) that has been engineered to express the active hK2 protein under a prostate-specific promoter. At tracer dose, the 11B6 imaging agent is able to define the two ventral lobes (which express the most protein).

FIG. 14C shows that crossing these transgenic animals with established models of prostate cancer, for example, this representative hK2×Hi-Myc mouse, yields greater uptake of the tracer in the cancerous prostate. Note that intact antibodies are excreted primarily through the liver, and therefore bladder signal is not expected or seen.

FIGS. 15A-15D show Cy5.5-11B6 cellular uptake.

FIG. 15A-15B show white light and fluorescence imaging of a single cell suspension of hK2-expressing mouse prostate after intravenously administered Cy5.5-11B6, respectively.

FIG. 15C shows confocal microscopy of cultured VCaP cells incubated with Cy5.5-11B6 overlaid on differential interference contrast light image of cells.

FIG. 15D shows three dimensional rendering of fluorescence distribution within the cells in XY (top) and YZ (bottom) perspectives.

FIGS. 16A and 16B show FcRn-specific transport.

FIG. 16A shows SPR determined dissociation constants for FcRn for 11B6 and H435A-11B6 at pH 6 and 7.4.

FIG. 16B shows exploiting the receptor's pH-dependent affinity, FcRn-mediated uptake is confirmed by increased uptake kinetics at low extracellular pH. Uptake is abrogated with H435A-modified 11B6.

FIGS. 17A-17F show investigation of FcRn-mediated uptake of 11B6 complexed with hK2.

FIG. 17A shows a comparison of uptake in LNCaP xenografts (and blood clearance from heart measurements) of the 11B6 antibody, and the single point mutated H435A-11B6.

FIG. 17B shows ex vivo organ and tumor biodistribution of antibody uptake at 320 h.

FIG. 17C shows in vitro verification of binding of both 11B6 and the H435A mutant to hK2 by immunofluorimetric competition assay.

FIG. 17D shows validation of the uptake of the intact antibody (11B6) and the lack of uptake of the antibody with an Fc-specific single amino acid point mutation (H435A) in hK2-expressing GEM (Pb_KLK2).

FIG. 17E shows ex vivo biodistribution of the two non-accumulating constructs (non-specific IgG1 and H435A) that demonstrates a requirement for both hK2 binding and FcRn internalization.

FIG. 17F shows biodistribution data at 320 h of hu11B6 and H435A.

FIG. 18 shows uptake of pH-dye labeled 11B6. Top row: 11B6, bottom row: control IgG. Prostate cancer cells (LNCaP) were pulsed with pH indicator dye-labeled antibody. Internalized 11B6 is not in an acidic environment at 12 h (but has been internalized; FIGS. 3A-3G). Fluorescence intensity increased in the acidic late endosomes at later time points. Control IgG was not detected.

FIGS. 19A-19G show imaging cross-activation of the AR pathway in LREX′ models.

FIGS. 19A-19E show biodistribution of —Zr-DFO-11B6 in flank xenografts of the enzalutamide-resistant LREX′ model in castrated animals with daily enzalutamide and dexamethasone treatment. A model of LREX′ liver metastasis was developed by orthotopic implantation of the cells in Matrigel in animals similarly supplemented with dexamethasone and enzalutamide. Metastasis burden was monitored by (FIG. 19B) bioluminescent imaging and (FIGS. 19C-19E) PET/MR using ⁸⁹Zr-DFO-11B6.

FIGS. 19F and 19G show H&E and autoradiography of the distribution of the tracer at metastatic deposits within the liver.

FIG. 20 shows accumulation of re-engineered anti-PSA antibody. Radiolabeled ⁸⁹Zr-antibody uptake in LNCaP flank tumors in nude mice. 5A10, an antibody targeting free PSA, experiences transient uptake in LNCaP xenografts (black, closed circles). When the CDR binding regions were grafted onto the 11B6 antibody scaffold and retaining free PSA specificity (5A10^{H435-wt}, it was observed that non-transient tumoral accumulation of the antibody (blue, open circles).

FIGS. 21A-21E show hK2 production after DHT stimulation. AR-positive breast cancer cell lines were found to secrete hK2 into the cell culture medium as detected by immunofluorimetric assay after DHT stimulation. The values for free hK2 (fhK2) for the positive cell lines (FIG. 21A) BT-474 and (FIG. 21B) MFM-223 are shown here without treatment (vehicle; VEH), with irrelevant hormone addition (estrogen; EST), and with testosterone (DHT). Note that the plots are on a log 10 scale. RT-PCR was performed on the cells to compare the expression of KLK2 and FOLH1 in (FIG. 21C) BT-474 and (FIG. 21D) MFM-223. FIG. 21E shows in vivo biodistribution of ⁸⁹Zr-11B6 in BT474 xenografts with estrogen and DHT stimulation.

FIG. 22 shows a PET/CT image of ⁸⁹Zr-DFO-11B6 in a subcutaneous MFM223 model following DHT stimulation, revealing the presence of AR+ triple negative breast cancer.

FIG. 23 shows intracellular accumulation of 11B6-hK2 in breast cancer cells. Under DHT stimulation, the AR-positive BT474 expresses hK2. Conjugated 11B6 is internalized in a time-dependent manner by the stimulated cells. Cy5.5-11B6, red; DAPI, blue.

FIGS. 24A-24E show quantitation of ⁸⁹Zr-11B6 uptake in transgenic PCa mice.

FIG. 24A shows ⁸⁹Zr-DFO-11B6 uptake in the transformed prostate was determined non-invasively by volume of interest measurement at baseline (ages 18-24 weeks).

FIGS. 24B-24D show ex vivo autoradiography and histology confirm prostate and tumor specific uptake.

FIG. 24E shows quantification of PET before and after castration.

FIGS. 25A-25E show serial PET/CT monitoring ⁸⁹Zr-11B6 uptake before, during, and after reversible castration by GNRH receptor blockade.

FIG. 25A shows treatment and ⁸⁹Zr-11B6 PET imaging schedule throughout testosterone-depleting degarelix therapy.

FIG. 25B shows initial PET/CT prior to treatment (12 weeks of therapy consisting of 2 consecutive depot injections of degarelix acetate).

FIGS. 25C-25E show representative images 2, 10, and 14 weeks after treatment initiation, respectively.

FIGS. 26A-26C show noninvasive monitoring of AR status with ⁸⁹Zr-DFO-11B6.

FIG. 26A shows relative expression of KLK2 in prostate tissue collected from Pb_KLK2 XHi-Myc mice without treatment, with castration and vehicle (saline) and with castration and adjuvant enzalutamide.

FIG. 26B shows PCR analysis of KLK2 gene expression in tissues treated with castration and enzalutamide resected by ⁸⁹Zr-DFO-11B6 guidance (shaded) and prostate tissues negative for 11B6 uptake (white).

FIG. 26C shows a plot of the amount of hK2 protein (normalized to the total protein concentration) of tissues from Pb_KLK2 XHi-Myc treated with full androgen blockade correlated with ⁸⁹Zr-DFO-11B6 uptake minimum (blue) and maximum (red) values. The plot shows that uptake quantified by PET correlates to the actual hK2 protein level.

FIGS. 27A-27N show multimodality imaging for pre- and intra-operative guidance and post-operative confirmation.

FIG. 27A shows volume-rendered PET/CT demonstrates localization of signal in the prostate for pre-operative planning.

FIGS. 27B-27G show white light (left), fluorescence (middle), and composite (right) images obtained at different stages during dissection of the prostate.

FIG. 27B shows detection of fluorescence corresponding to prostate lobes through an intact peritoneum and abdomen.

FIG. 27C shows fluorescence signal outlines the hK2 positive tissue of the intact ventral prostate lobes.

FIG. 27D shows a post-hemiectomy: an intact right ventral prostate lobe after left lobe removal.

FIG. 27E shows imaging after gross removal of both ventral lobes. Bladder indicated with (*).

FIG. 27F shows delineation of intact dorsal-lateral lobes after rostral/caudal manipulation of the bladder (*).

FIG. 27G shows stereoscope magnification (ruler separations are approximately 800 μm) of area outlined in E. The resected prostate lobes imaged with (FIG. 27H) conventional white light, (FIG. 27I) fluorescence, and (FIG. 27J) radio-signal.

FIG. 27K shows a post-surgical PET/CT reveals a small remnant focus of signal (arrow). After excision at autopsy, seminal vesicles, urethra, and remnant tissue were sectioned and imaged by (FIG. 27L) autoradiography and (FIG. 27M) fluorescent microscopy.

FIG. 27N shows a hematoxylin-eosin stain confirmed adenocarcinoma.

FIG. 28 shows ¹⁸F-FDHT imaging. The distribution at 1.5 h after administration in a representative VCaP (arrow) bearing mouse. ¹⁸F-FDHT is a radiolabeled analog of the non-aromatizable dihydrotestosterone. Liver, bile and kidney uptake is equivalent to or exceeds tumor uptake.

FIGS. 29A-29B show pathological analysis of sections of GEM model of disease after castration.

FIG. 29A shows 10 μm sections of tissue that did not demonstrate uptake of 11B6 imaging probe after castration.

FIG. 29B shows sections from a 11B6 signal-positive tissue. 10× micrographs (scale is 500 μm) with 40× insert (scale is 50 μm). Clockwise from top left: staining for androgen receptor, Ki-67, haemotoxylin and eosin and c-MYC.

FIG. 30 shows concordance between quantitative ex vivo imaging and protein content. Uptake of ⁸⁹Zr-DFO-11B6 on PET (measured as % IA/g in volumes of interest from PET imaging) in individual prostate lobes correlated to tissue content of hK2 (normalized to the total protein concentration). R² is 0.9928.

FIG. 31 shows comparison of human and murine 11B6. Humanization of the antibody did not affect the binding and uptake of radiolabeled antibody in xenograft models of prostate cancer (LNCaP; n=4). Serial microPET images were analyzed for uptake at the tumor site and in the blood (assessed from manually delineated volumes of interest of the xenograft and heart, respectively), and mean volume of interest values are presented as % IA/g.

FIGS. 32A-32D show h11B6 immunohistochemistry. To evaluate 11B6 binding of kallikrein-related peptidase (free hK2), application of the murine 11B6 antibody with an anti-rodent secondary antibody to human prostate and prostate cancer biopsy specimens. Hematoxylin counterstained specimens showed the glandular structure of the prostate and hK2 distribution in the prostatic alveoli and intraluminal secretions of representative samples, including (FIG. 32A) the normal prostate, (FIG. 32B, 32C) two representative prostate tumor tissue specimens, and (FIG. 32D) metastatic foci (lesion in the bone). Scale bar in 4× images is 250 μm, in 40× inserts it is 50 μm.

FIGS. 33A-33B show schematic representation of prostate-specific active hK2 in a genetically engineered KLK2 expressing mouse model.

FIG. 33A shows a schematic representation of the generation of the Furin protease activated pre-pro-hK2 GEM to yield a prostate-specific, catalytically active hK2 in vivo. Insertion of a Furin cleavage site sequence upstream of the catalytic region of pre-pro-hk2.

FIG. 33B shows that the Furin protease cleavage site is selectively severed by prostate-specific Furin expression, releasing catalytically active hK2.

FIGS. 34A-34B show genotyping data.

FIG. 34A shows a southern blot of BAMHI-digested samples from control (lane annotated WT), and transgenic mice hybridized with a 2.3 Kb Probasin-fur-hK20-SV40 site probe (annotated 43). This positive founder was used for further breeding. The size markers on the right correspond to BAMHI digested fragments of lambda Hind III, at a dilution corresponding to 10 copies (annotated 10C).

FIG. 34B shows PCR evaluation of candidate transgenic and control mice for the incorporation of FurhK2 cDNA (upper bands) and GAPDH cDNA (bottom bands) levels indicated equal loading. Lane numbers refer to individual genotyped animals. Sample number 25 correlates to the selected mouse for further breeding (Founder line 43). Controls include non-crossed animal (annotated 17), HK2 spiked (31) and FurinhK2 spiked (32) wild type animals. Invitrogen 100 base pair ladder shown at right.

### DETAILED DESCRIPTION

It is contemplated that systems, methods, and compositions of the present disclosure encompass variations and adaptations developed using information from the embodiments described herein. Adaptation and/or modification of the systems, methods, and compositions described herein may be performed by those of ordinary skill in the relevant art.

Throughout the description, where systems are described as having, including, or comprising specific components, or where processes and methods are described as having, including, or comprising specific steps, it is contemplated that, additionally, there are systems of the present disclosure that consist essentially of, or consist of, the recited components, and that there are processes and methods according to the present disclosure that consist essentially of, or consist of, the recited processing steps. Moreover, where compositions are described as having, including, or comprising specific components, it is contemplated that, additionally, there are compositions of the present disclosure that consist essentially of, or consist of, the recited components.

It should be understood that the order of steps or order for performing certain actions is immaterial so long as the process remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Subject headers are provided herein for convenience only. They are not intended to limit the scope of embodiments described herein.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., Intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time. As is known in the art, antibody therapy is commonly administered parenterally (e.g., by intravenous or subcutaneous injection).

“Biomarker”: The term “biomarker” is used herein, consistent with its use in the art, to refer to a to an entity whose presence, level, or form, correlates with a particular biological event or state of interest, so that it is considered to be a “marker” of that event or state. To give but a few examples, in some embodiments, a biomarker may be or comprises a marker for a particular disease state, or for likelihood that a particular disease, disorder or condition may develop. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be an entity of any chemical class. For example, in some embodiments, a biomarker may be or comprise a nucleic acid, a polypeptide, a lipid, a protein (e.g., an antibody), a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc.

“Cancer”: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. Cancer cells include precancerous (e.g., benign), malignant, pre-metastatic, metastatic, and non-metastatic cells.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Marker”: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

“Peptide” or “Polypeptide”: The term “peptide” or “polypeptide” refers to a string of at least two (e.g., at least three) amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/^{˜}dadgrp/Unnatstruct.gif, which displays structures of non-natural amino acids that have been successfully incorporated into functional ion channels) and/or amino acid analogs as are known in the art may alternatively be employed). In some embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Radiolabel” or “Radionuclide”: As used herein, “radiolabel” or “radionuclide” refers to a moiety comprising a radioactive isotope of at least one element. Exemplary suitable radiolabels include but are not limited to those described herein. In some embodiments, a radiolabel is one used in positron emission tomography (PET). In some embodiments, a radiolabel is one used in single-photon emission computed tomography (SPECT). In some embodiments, radioisotopes comprise ^{99m}Tc, ¹¹¹In, ⁶⁴Cu, ⁶⁷Ga, ⁶⁸Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁸⁶Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, ⁸²Rb, ²¹²Bi, ²¹³Bi, and ¹⁹²Ir.

“Sample”: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is or comprises a tumor, tumor tissue, or tumor cells. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

“Subject”: As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment can be of a subject who does not exhibit signs of the relevant disease, disorder and/or condition and/or of a subject who exhibits only early signs of the disease, disorder, and/or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, and/or condition.

Targeting the androgen receptor (AR) pathway by receptor blockade and androgen depletion prolongs survival in patients with prostate cancer and a subset of breast cancers, but drug resistance rapidly develops. Understanding this resistance is confounded by a lack of non-invasive means to assess AR activity in vivo. Here is presented an approach involving intracellular accumulation of a secreted antigen targeting antibody (SATA) for disease characterization and therapy. AR-regulated human kallikrein-related peptidase (free-hK2) is a prostate tissue-specific antigen produced in prostate cancer and androgen-stimulated breast cancer cells. Fluorescent and radio-conjugates of 11B6, an antibody targeting free-hK2, are internalized and non-invasively report AR-pathway activity in metastatic and genetically engineered models of cancer development and treatment. Uptake is mediated by a previously unrecognized mechanism involving the neonatal Fc-receptor. The technology described herein transforms the current antibody landscape by demonstrating cell-specific SATA uptake for diagnosis and therapy in other cancers and/or metastases.

Presented herein are systems, methods, and compositions involving the use of murine and/or humanized antibodies targeting free PSA (such as 5A10) and/or free hK2 (such as 11B6) for in vivo targeting of androgen receptor (AR) positive cancer (e.g., breast cancer, e.g., prostate cancer). For example, the antibodies can be used alone (e.g., 5A10 or 11B6) or in combination (e.g., 5A10 and 11B6).

For example, in certain embodiments, the present disclosure is directed to immuno-PET/SPECT and/or immuno-fluoresce-guided imaging for diagnosing, localizing, radiation dose planning, and/or evaluating therapy response (e.g., anti-androgen receptor therapeutics, surgery and external irradiation) in androgen receptor (AR) positive breast cancer or PCa. Evaluation can include monitoring of AR-upregulation of KLK2 and KLK3 in response to external irradiation.

In other embodiments, for example, the present disclosure is directed to radio-immunotherapy (RIT) treatment of AR-positive breast cancer by administration (e.g., injection) of a free-PSA and/or free hK2 antibody labelled with a radioisotope after KLK2 and KLK3 induction by progesterone, testosterone or irradiation.

In other embodiments, for example, the present disclosure provides an antibody-based platform directed to a secreted antigen that uses Fc-receptor mediated internalization for cancer imaging and therapy.

In other embodiments, a new approach is described herein using an antibody (e.g., 11B6) directed to an epitope accessible only on the free, catalytically active form of human kallikrein-related peptidase 2 (hK2). When 11B6 is bound to active hK2, this complex is permanently internalized and transported to lysosomal compartments. Despite the homology similarity between the kallikreins, 11B6 is specific for hK2 and does not bind PSA. Humanized IgG1 11B6 internalizes and accumulates in BCa cells that express human kallikrein 2 (hK2), which is only expressed when the AR-axis is active.

The antibody 11B6 specifically binds to an epitope in the catalytic pocket of hK2 that is blocked by protease inhibitors when the enzyme is shed/released into the blood circulation. By labeling humanized 11B6 (hu11B6) with the positron emitting radio-metal Zirconium-89 (⁸⁹Zr), or other compounds that can be detected by PET or SPECT, presented herein is an immune-imaging platform (hu11B6) that quantitatively detects presence and activity of the AR axis in BCa. In addition, experiments are also described in which 11B6 is labeled with Actinium-225 (²²⁵Ac), an alpha particle chemical element, for therapeutic applications.

Uptake of 11B6 is observed in free hK2-producing cancer cells, as described in the experiments presented herein, in a process facilitated by the neonatal Fc receptor (FcRn). This feature is recapitulated in AR-positive BCa models treated by hormones which stimulate hK2 production. Therefore, the present disclosure demonstrates a unique ability to profile and monitor AR activity in two commonly diagnosed non-cutaneous cancers, PCa and some types of BCa.

In certain embodiments, treatment methods are presented including administration of ²²⁵Ac-DOTA-hu11B6 in combination with induction of the AR-axis by progesterone treatment, testosterone treatment, and/or external irradiation, for better therapeutic effect.

In certain embodiments, 11B6 is applied for both positron emission tomography (PET) and fluorescent imaging in xenograft and genetically engineered models for disease detection to 1) quantitatively assess AR pathway activity, 2) determine pharmacodynamic parameters, and 3) evaluate treatment efficacy in immunocompetent models, and 4) guide treatment in clinically relevant scenarios.

11B6 immunoimaging resolves issues at key clinical decision points for both prostate and breast cancer patients to significantly improve management. Also, it is shown herein that the FcRn mediated uptake mechanism can be exploited to facilitate uptake by other SATA. There does not appear to be any previous report of targeted tissue specific uptake of a secreted antigen; thus, the technology described herein provides a new strategy for precision imaging of disease processes.

Results: Uptake of hK2-Targeting ⁸⁹Zr-11B6 Radiotracer Correlates to Expression Level of its Target Enzyme

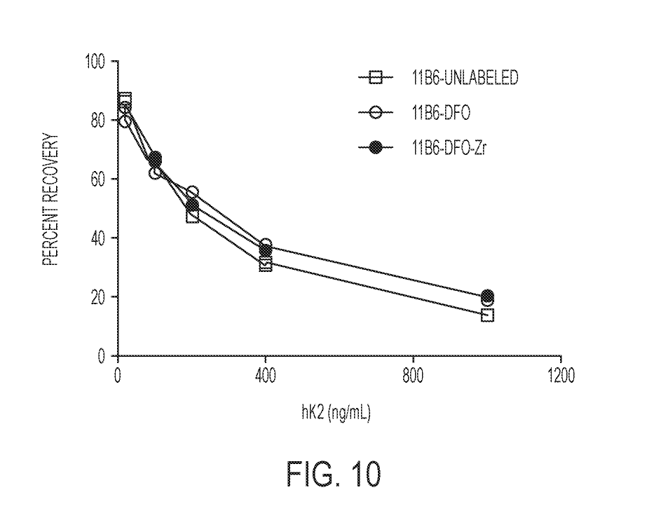

Studies have revealed that hK2 is an anatomically and disease restricted protein. Described herein is a generated murine antibody, 11B6, with specificity for the catalytic pocket of free hK2. Conjugated to desferrioxamine B (DFO) and subsequent Zirconium-89 (⁸⁹Zr) labeling yielded ⁸⁹Zr-11B6, a positron emission tomography (PET) radiotracer. A competition binding assay was conducted revealing that bioconjugation of 11B6 resulted in no significant loss of affinity for hK2 (FIG. 10). In vitro studies of ⁸⁹Zr-11B6 uptake showed expression-specific uptake, and specificity was verified by blocking with excess 11B6. Activity after washing revealed this SATA was internalized by hK2 expressing cells.

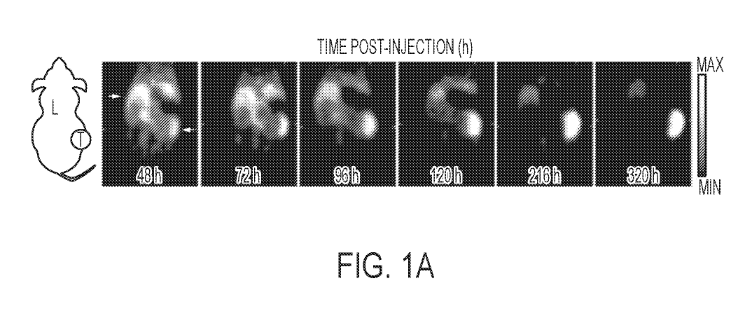

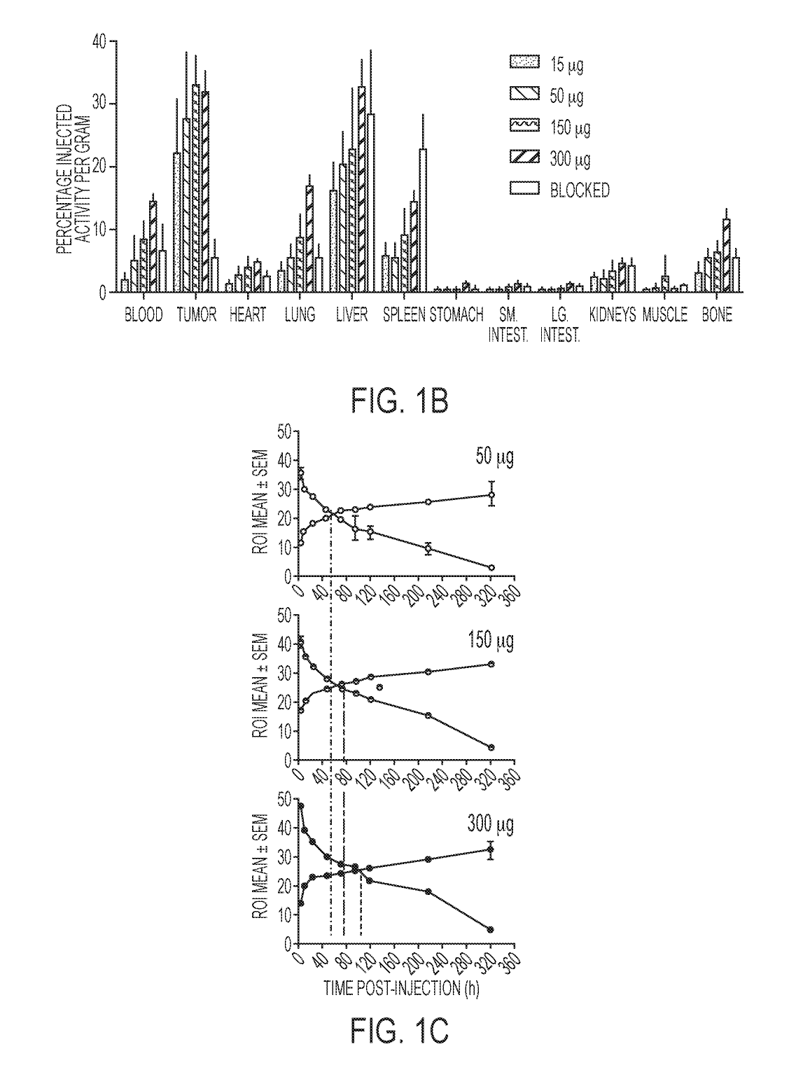

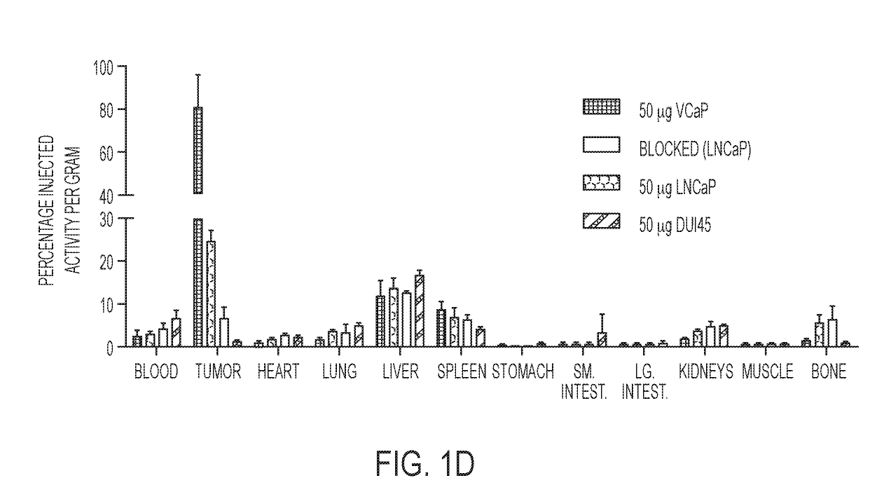

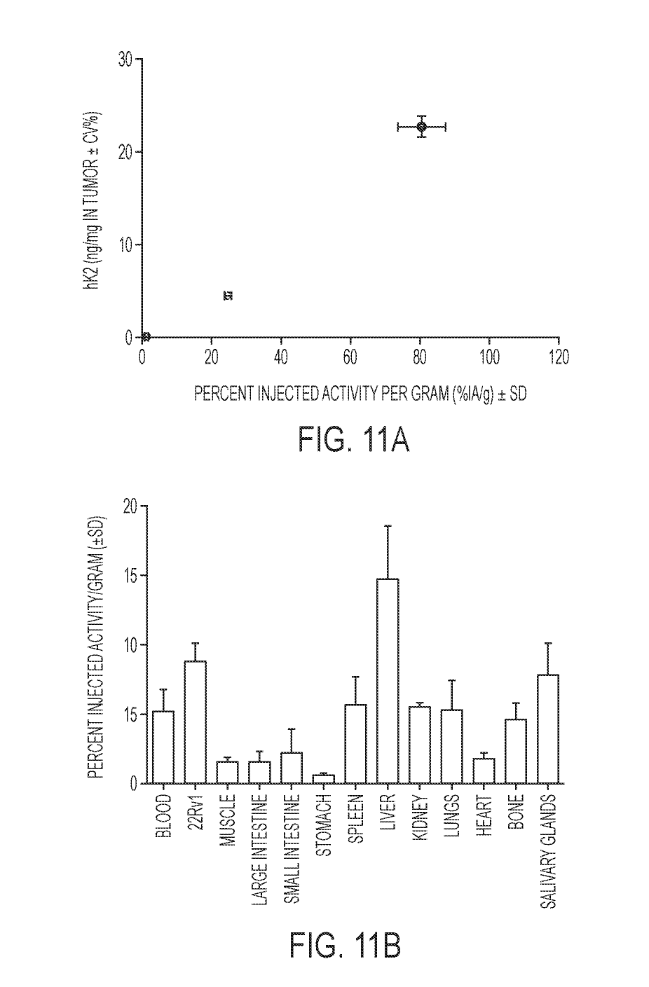

To test if uptake occurred in vivo, the dose of ⁸⁹Zr-11B6 was first optimized with an escalation study and PET quantification in mice bearing human prostate cancer xenografts (FIGS. 1A-1D). The time to tumor saturation inversely correlated with tracer mass and improved tumor/blood contrast; 2.4, 4.2, 7.7 and 13.7 hours for 300, 150, 50, and 15 μg of ⁸⁹Zr-11B6, respectively. Subsequent experiments utilized 50 μg ⁸⁹Zr-11B6, which achieved tumor saturation with low background activity after 120 hours (FIG. 1C). In vivo specificity was verified by blocking (1 mg, unlabeled-11B6) and using hK2-negative DU145 xenografts. To assess the potential for imaging of patients that have failed hormone therapy, uptake was measured in castration resistant 22Rv1 tumor xenografts, as well. Here, it was observed that there was robust localization to the tumor through continued AR-driven hK2 expression (FIG. 11).

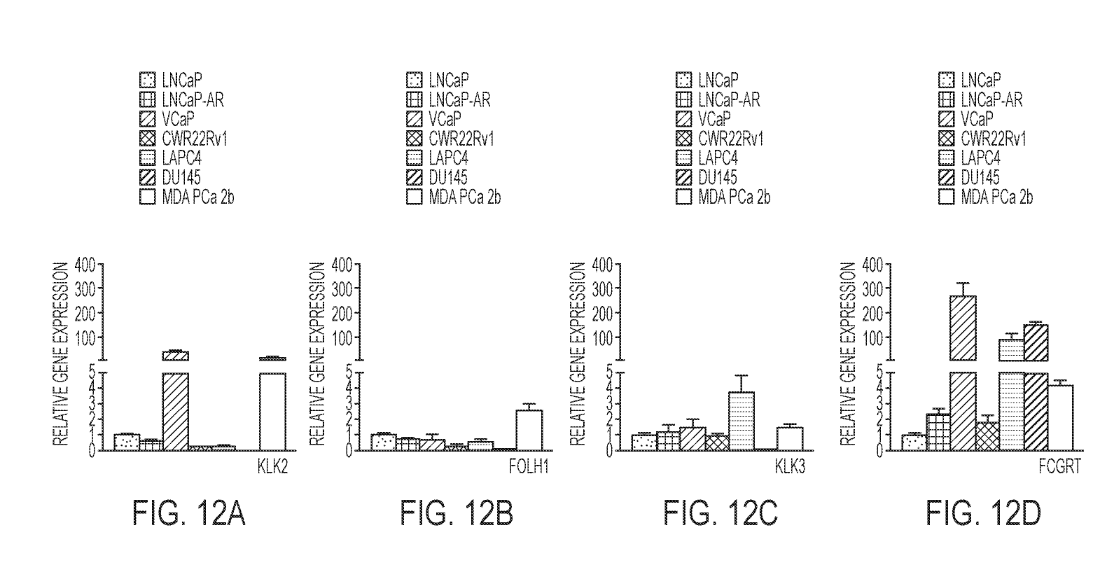

KLK2 expression was evaluated in 7 xenograft lines (FIG. 12A). VCaP exhibited the highest KLK2 levels and showed markedly higher ⁸⁹Zr-11B6 internalization (80.7 percent injected activity per gram (% IA/g)) compared to LNCaP (24.7% IA/g) (FIG. 1D), demonstrating the ability to determine hK2 expression status in vivo. The expression level of KLK2 did not correlate with two other AR-governed imaging targets, KLK3 (PSA) or FOLH1 (PSMA), underlining the use of hK2 as a distinct biomarker (FIGS. 12A-12D).

### Delineating Bone Metastases

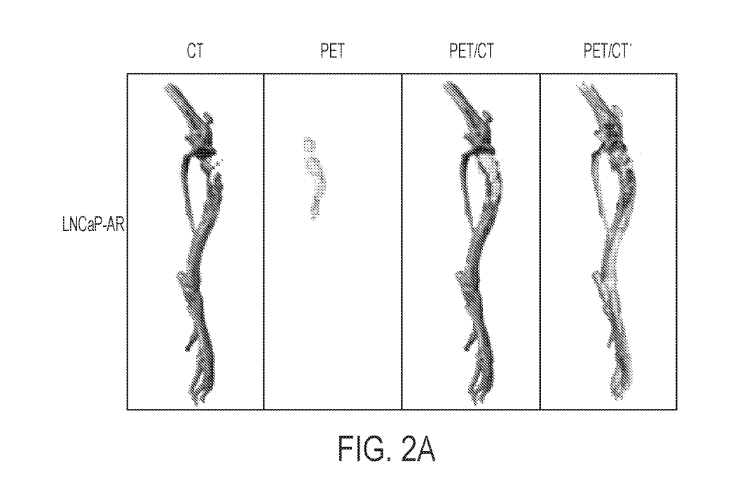

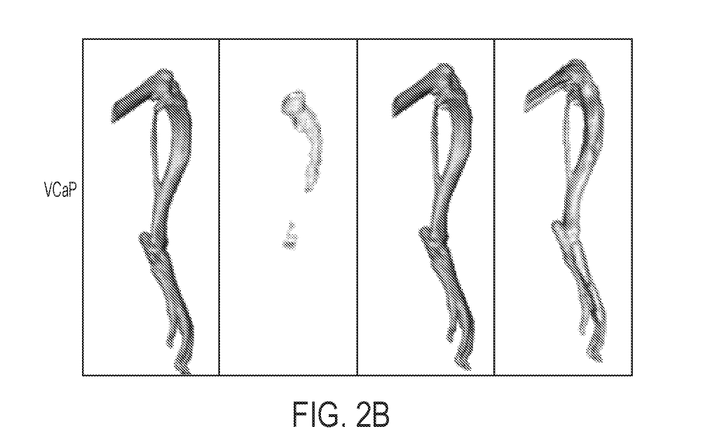

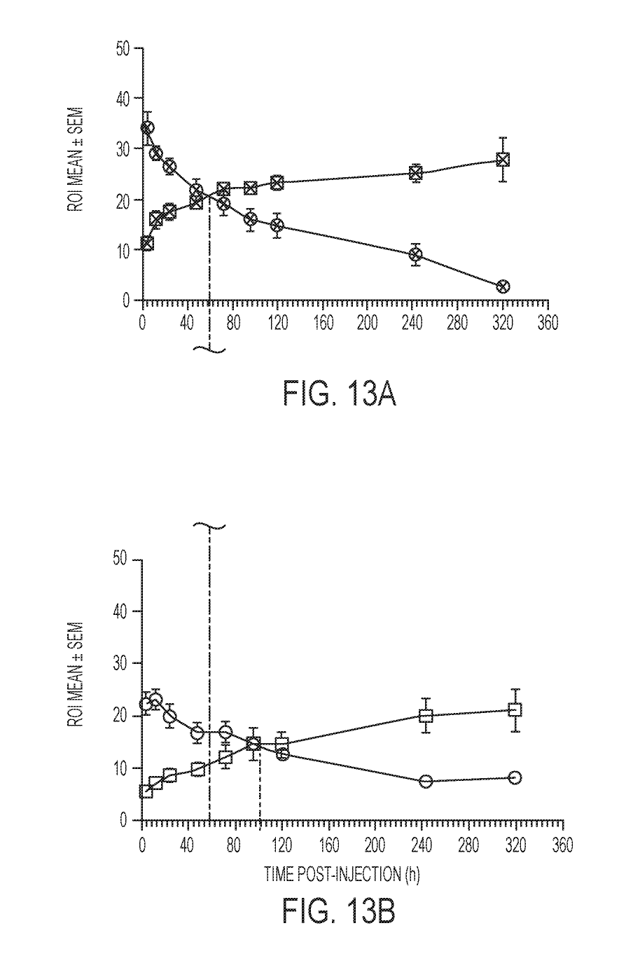

Bone is a common site of PCa and BCa metastasis, often manifesting a mixed bone forming/resorbing phenotype that complicates detection by current clinical imaging methods, which rely on the uptake at sites with increased osteoblastic activity. The ability of ⁸⁹Zr-11B6 to detect both phenotypes was evaluated using intraosseous LNCaP-AR (osteolytic) and VCaP (osteoblastic) bone metastases models with control PC3 (AR/hK2 negative osteolytic) bone lesions (FIGS. 2A-2C). ⁸⁹Zr-11B6 PET demonstrated robust delineation in both osteometastatic phenotypes of AR-positive disease, with uptake delayed relative to subcutaneously inoculated tumors (FIGS. 13A-13B).

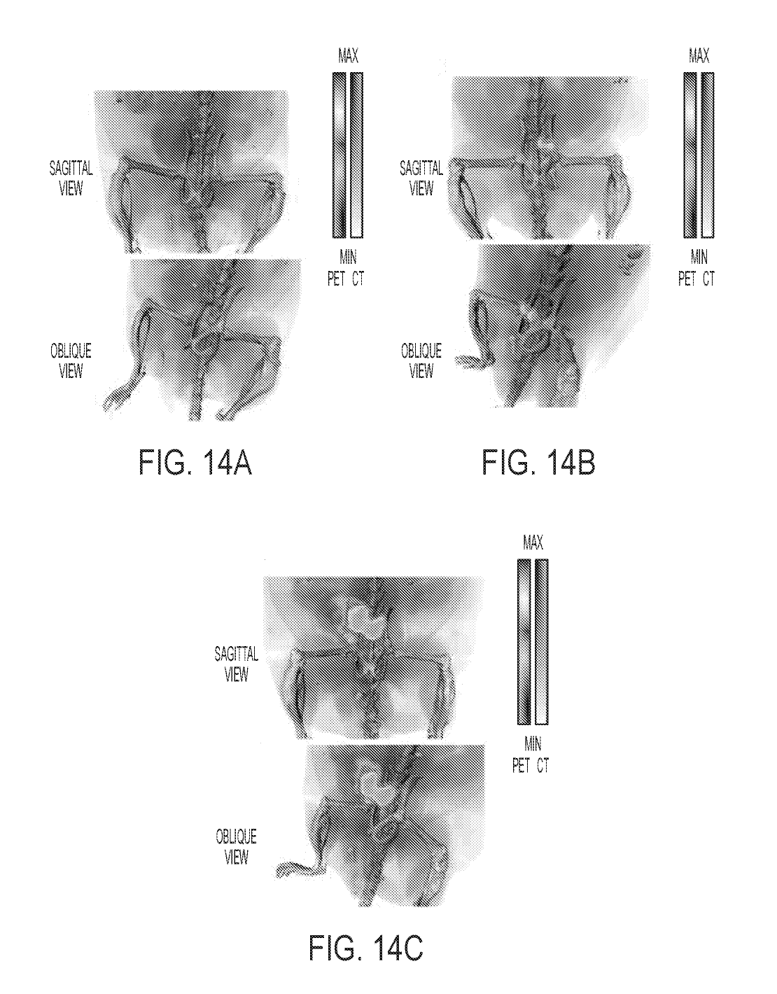

Faithful recapitulation of PCa for study in mice is particularly difficult given the absence of murine orthologs of several human prostate specific genes, including the prostate kallikreins. To test tracer kinetics in an immunocompetent milieu and measure uptake in autochthonous mouse tumors, a prostate full-length KLK2 construct encoding pre-pro-hK2 was cloned under control of the probasin promoter (Pb_KLK2), enabling prostate-specific and androgen driven expression of hK2. Using B6 mice as negative controls, ⁸⁹Zr-11B6 uptake was specific to hK2 positive prostatic tissue in vivo (FIGS. 14A-14C).

### 11B6-hK2 is Internalized by FcRn

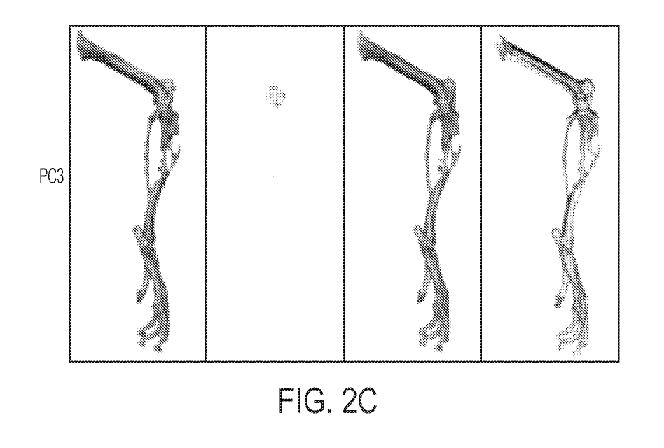

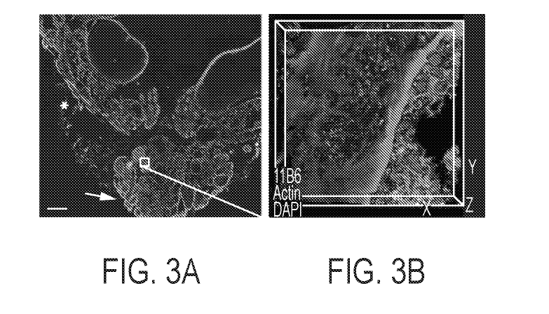

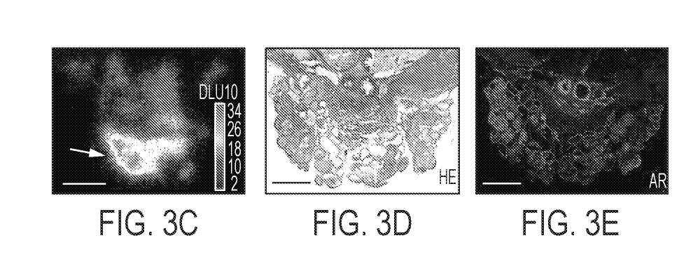

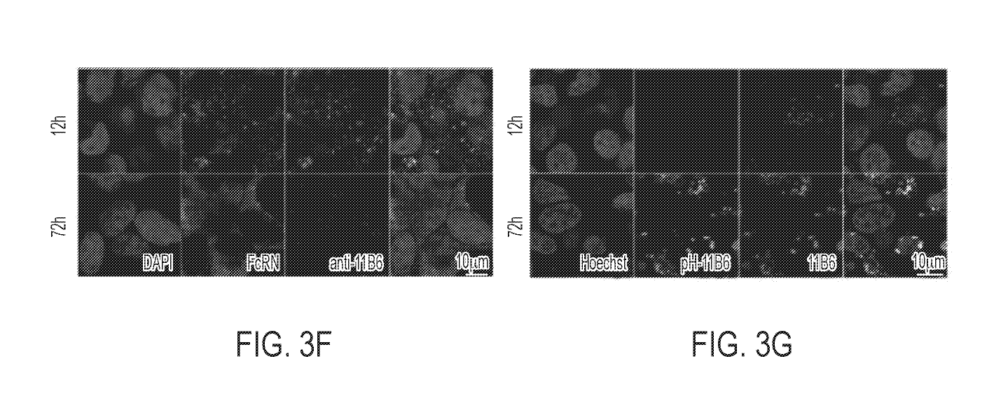

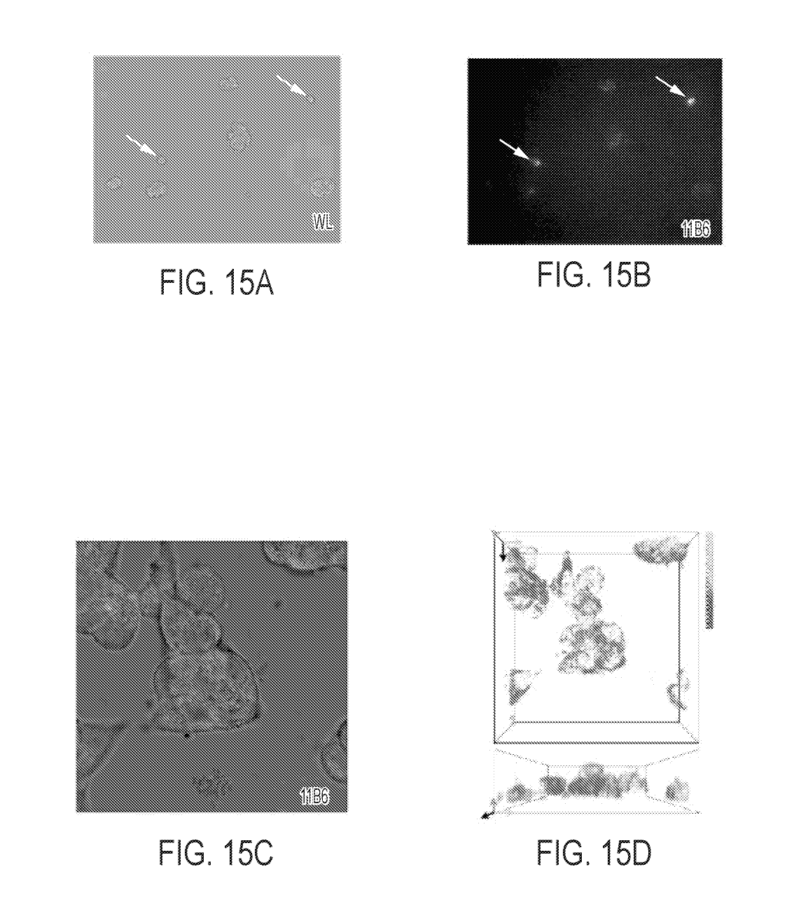

To investigate SATA internalization, conjugated 11B6 was first evaluated in whole-mount sections of prostate tissue from Pb_KLK2 mice. A high concordance between intravenously administered fluorescent and radioactive tracer was observed, as was an association between antibody uptake and staining for AR (FIGS. 3A-3E). 11B6 in the lumen of prostatic ducts suggested uptake by epithelial cells, confirmed by confocal microscopy (FIG. 3B). To verify, single cells extracted from this tissue were analyzed for fluorescent antibody uptake. In addition, analysis of PCa cell lines was performed in vitro (FIGS. 15A-15D).

The neonatal Fc receptor (FcRn) generally facilitates antigen recognition in luminal structures throughout the body and is expressed in a large set of PCa lines (FIG. 12D). Intracellular transport of the conjugate was determined by co-staining prostate cancer cells for FcRn and anti-IgG. Following pulsed exposure, 11B6 is associated with FcRn during the early phase of uptake. At late time-points, 11B6 appears intracellularly, and FcRn returns to the cell membrane. The 11B6-hK2 complex is shuttled from physiological pH early-endosomes to acidic late-endosomes, as shown using a pH-responsive dye conjugated to 11B6 and imaged in live cells (FIGS. 3F, 3G).

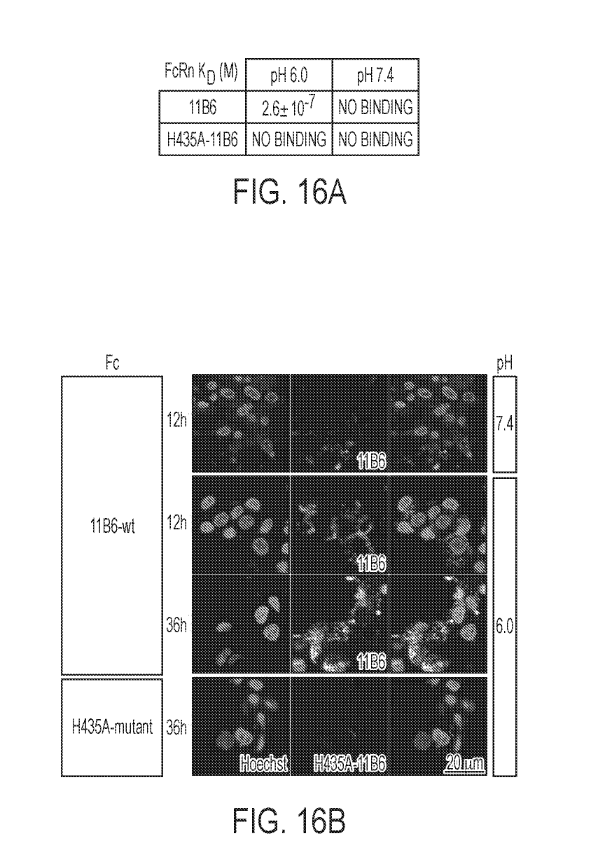

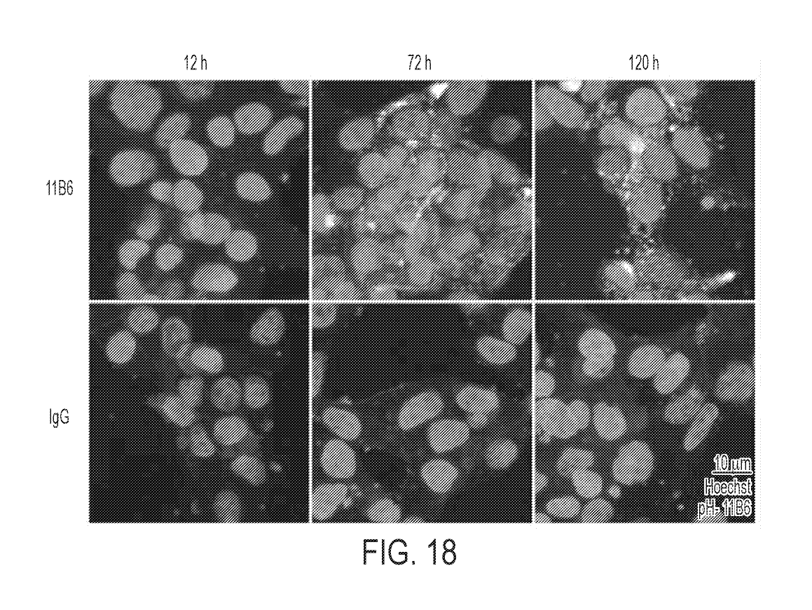

To confirm the specific role of FcRn in internalization of the SATA-antigen complex, recombinant mutant-11B6 IgG₁ was generated (modified at Histidine 435 to Alanine; H435A-11B6) to abrogate FcRn binding and cellular uptake was compared in physiological and acidified media. This mutation abrogates FcRn binding but does not affect variable region recognition or affinity. Surface plasmon resonance affinity of FcRn for 11B6 was pH dependent, and absent in the H435A-11B6 mutant (FIG. 16A). In culture, incubation at acidic pH conditions, as found in tumors and the prostate, augmented internalization (FIGS. 16A-16B). The presence of both FcRn and hK2 was required for internalization, and isotype matched control antibody did not bind cells or transport intracellularly via endosomes (FIG. 18).

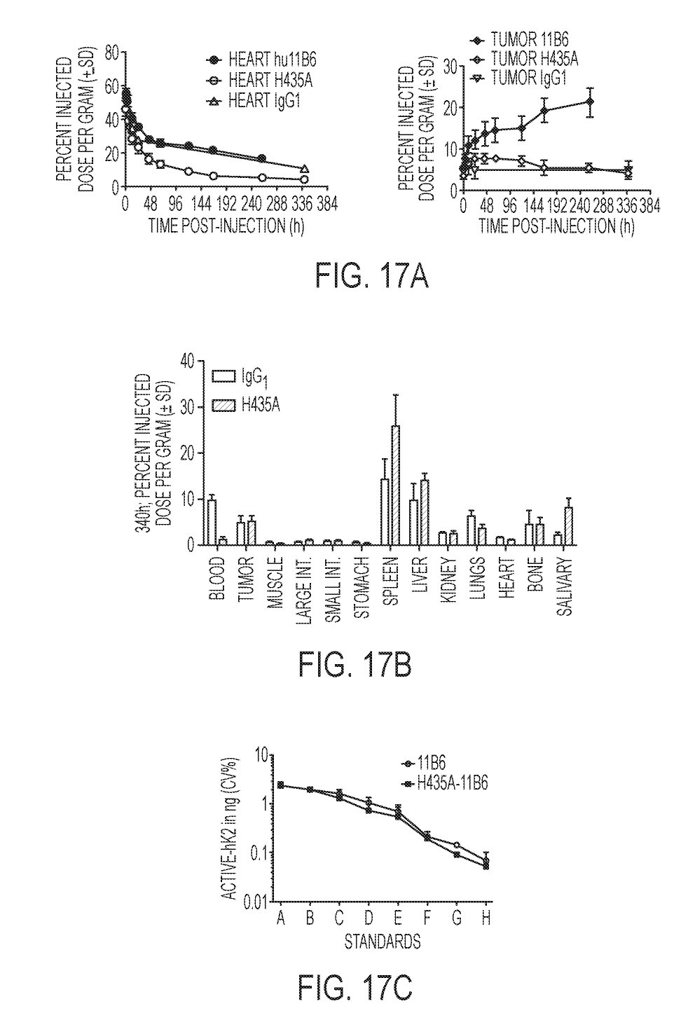

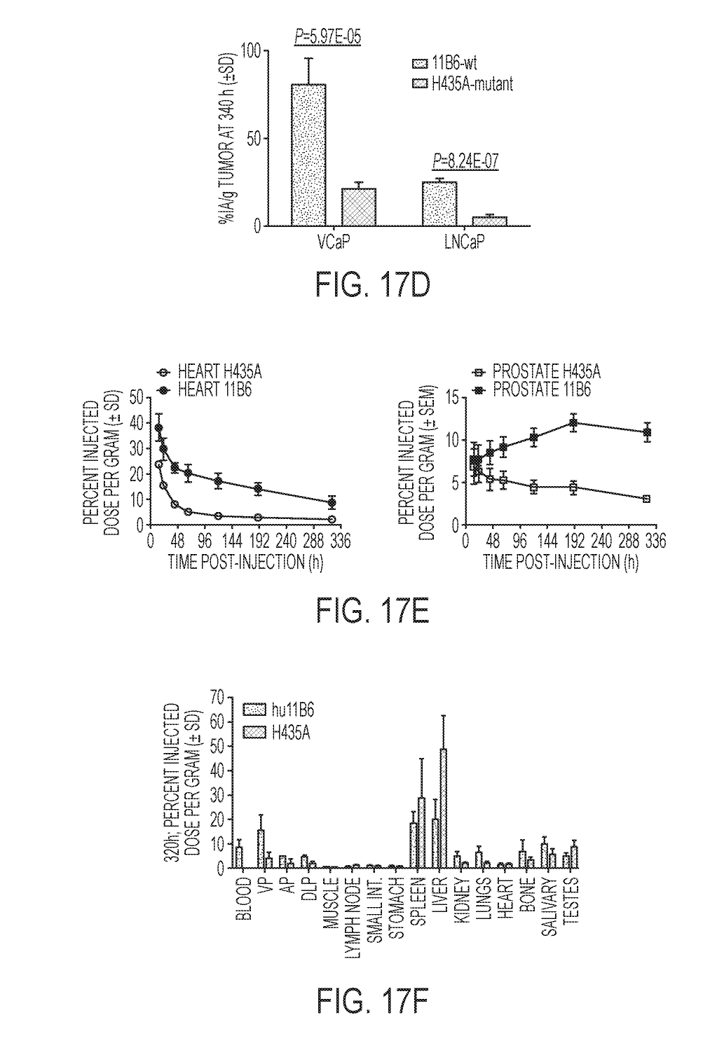

⁸⁹Zr-labeled 11B6 and mutant-Fc antibody were applied to establish FcRn dependence in vivo. Uptake of the mutant-Fc antibody matched that of control non-specific IgG (shown for LNCaP; (FIGS. 17A-17B), despite retained immunoreactivity of the FcRn binding-deficient antibody (FIG. 17C). Relative to the wild-type 11B6 antibody, H435A-11B6 uptake in immunodeficient xenograft models was significantly lower (21.2% IA/g for VCaP, P=5.97E-5; and 5.23% IA/g for LNCaP, P=8.24E-7); (FIG. 17D). Immunocompetent GEMM of adenocarcinoma (obtained by crossing Pb_KLK2 with ARR2/probasin-Myc (Hi-Myc), accumulate 11B6 in the transformed lobe of the prostate, while uptake of FcRn-binding deficient H435A-11B6 was abolished (FIGS. 17A-17E).

FcRn is widely expressed in tissues throughout the body, and particularly concentrated in the liver. Antibody imaging in this organ is difficult as non-specific uptake and clearance increase background. Thus, in addition to the demonstration of changes in uptake in GEM presented herein (FIGS. 17A-17E), it was desired to test if metastasis of PCa to the liver could be identified, an end-stage site of disseminated disease. ⁸⁹Zr-11B6 PET and magnetic resonance imaging revealed specific focal accumulation in hK2-expressing LREX′ metastases in the liver that were resistant to enzalutamide, a second-generation AR antagonist (FIG. 18). Autoradiography and histopathological findings correlate with the noninvasive assessment, demonstrating that targeting a secreted target downstream of central PCa biology is able to quantitate incipient resistance.

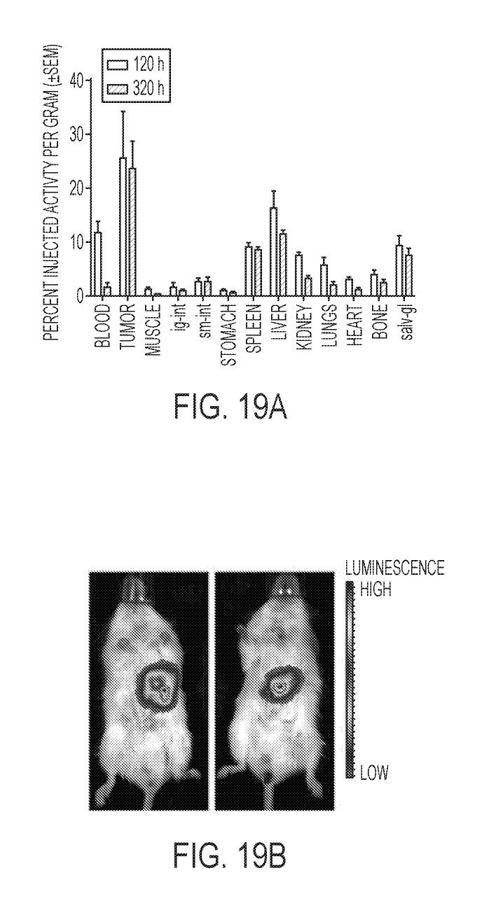

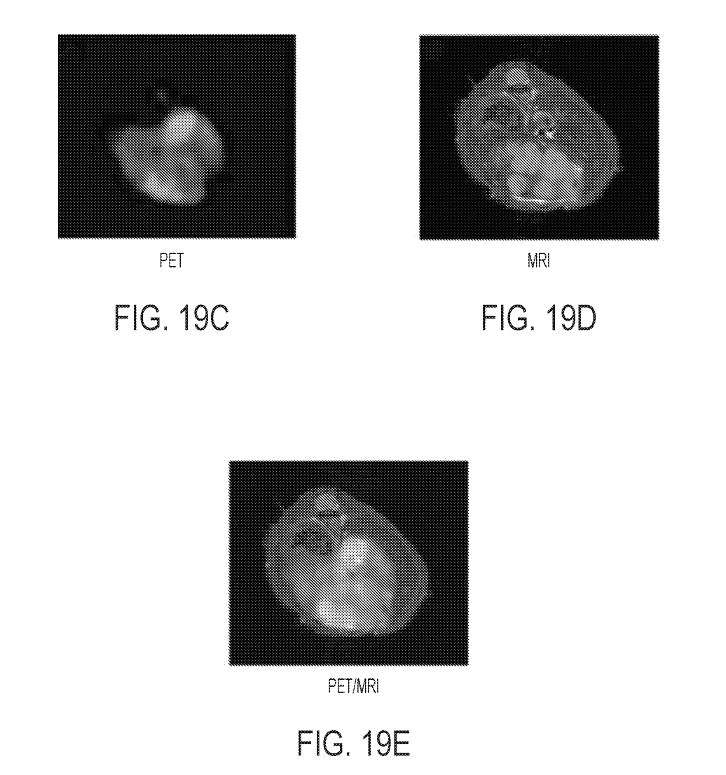

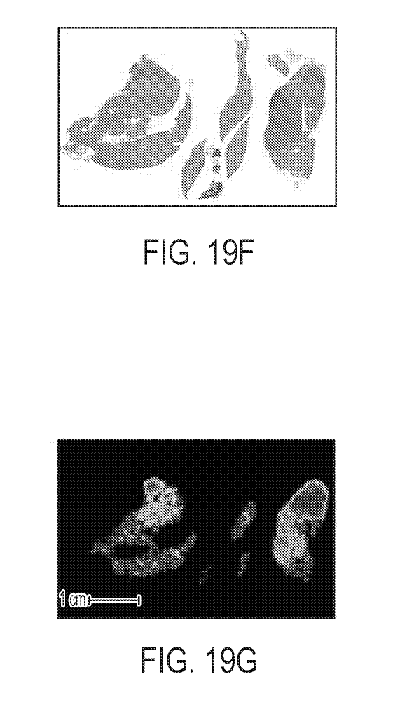

It was also tested whether uptake of antibody-secreted antigen complexes could be applied to other targets. Previously, it was shown that targeting free-PSA with an antibody (5A10) can delineate subcutaneous xenografts (for example, U.S. Pat. No. 8,663,600 describes a method involving injection of tracer-labelled antibodies, and visualizing PSA-producing or hK2-producing tissue for diagnosis of prostate cancer). Transient uptake was observed, as the SATA-antigen complex was not internalized and washed out of the tumor microenvironment. Previously identified residues at the constant heavy chain 2 and 3 (CH2/CH3) junction contribute to the pH-dependent affinity of the IgG interaction with FcRn, yielding multiple options for the inability of 5A10 to bind FcRn. The complementarity determining regions (CDRs) of 5A10 were grafted onto the 11B6 Fc-scaffold (bearing the histidine at residue 435; 5A10^{H435-wt}) In vivo, steadily increasing tumor uptake was observed using ⁸⁹Zr— 5A10^{H435-wt} in LNCaP xenografts, in contrast to the original PSA-targeting 5A10 (FIGS. 19A-19G).

### Antibody-hK2 Uptake in AR⁺ Breast Cancer

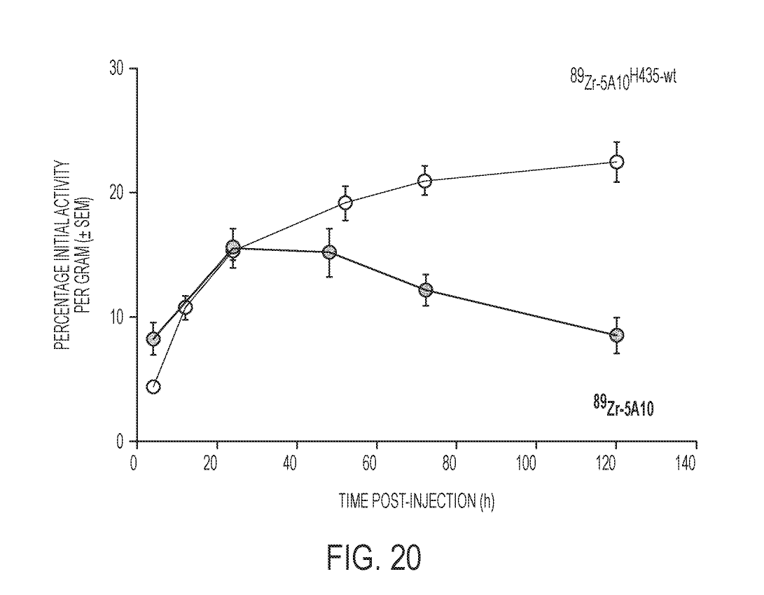

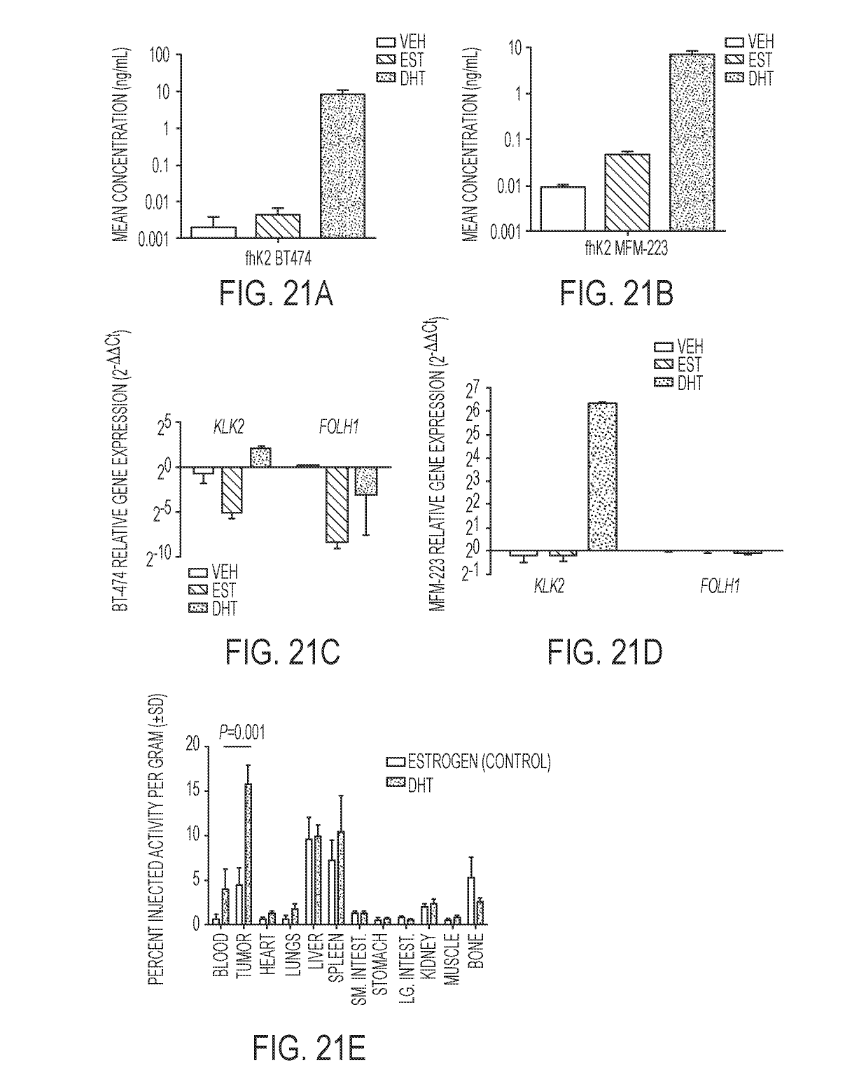

KLK2 expression is restricted to the prostate and PCa tissues in man, however it has been demonstrated that hK2 and PSA are detectable in (female) BCa cell lines and primary patient samples after appropriate activation of the AR-pathway by steroid hormones. Experiments were performed to investigate whether FcRn-mediated internalization of the antibody-bound hK2 is prostate specific. Under dihydrotestosterone (DHT), a subset of AR-positive BCa lines secrete hK2, including the triple-negative BCa line MFM-223 (FIG. 20). Androgen stimulation increased the AR-responsive KLK2 (FIGS. 21C-21D).

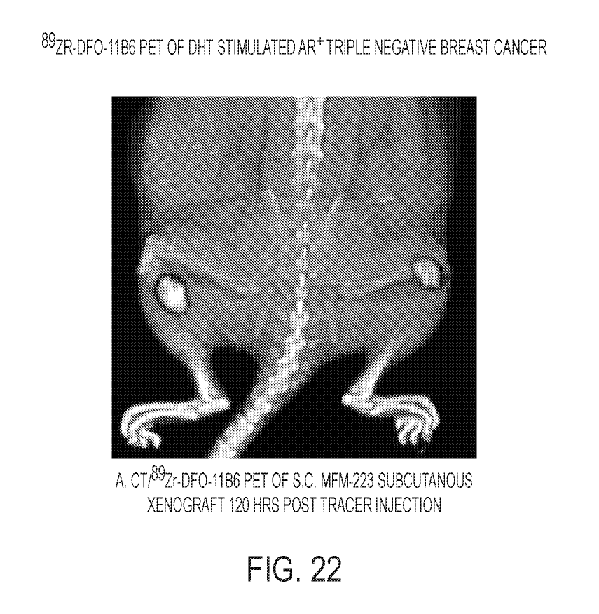

It was assessed whether 11B6 is internalized in a non-prostate derived cancer model. ⁸⁹Zr-11B6 was used to image AR-positive BCa with BT474 (ER⁺/PR⁺/HER2⁺/AR⁺) xenografts. ⁸⁹Zr-11B6 uptake was significantly greater in DHT treated female mice, compared to estrogen alone (P=0.001, FIG. 19E). As above, confocal microscopy reveals BT474 cells with and without DHT treatment internalize 11B6 in a time dependent manner (FIGS. 21A-21E). FIG. 22 is a PET/CT image of ⁸⁹Zr-DFO-11B6 in a subcutaneous MFM223 model following DHT stimulation, revealing the presence of AR+ triple negative breast cancer.

### Staging Adenocarcinoma and Monitoring Treatment

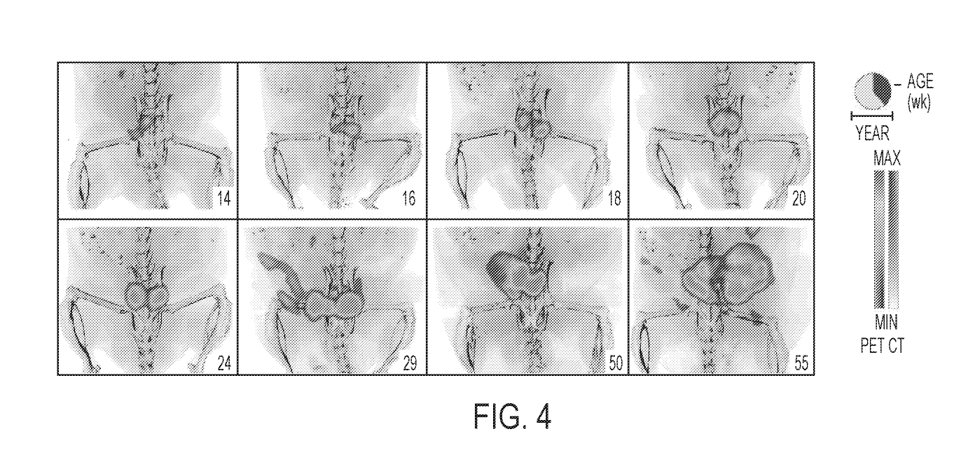

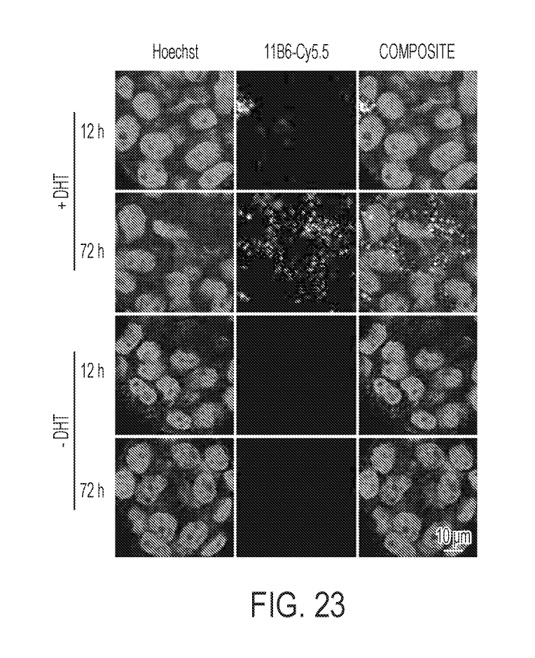

Next, ⁸⁹Zr-11B6 PET was applied to detect and monitor tumor progression in the prostate of transgenic models of adenocarcinoma (FIG. 4). Greater SATA uptake at sites of disease is noted, demonstrating heterogeneous progression, even at the small scale of the mouse prostate. Quantitation of tracer accumulation in the prostate corresponded with transformation from prostatic intraepithelial neoplasia through to adenocarcinoma. Ex vivo autoradiography of tracer microdistribution and histological adenocarcinoma is shown for a 50-week-old mouse (FIG. 23).

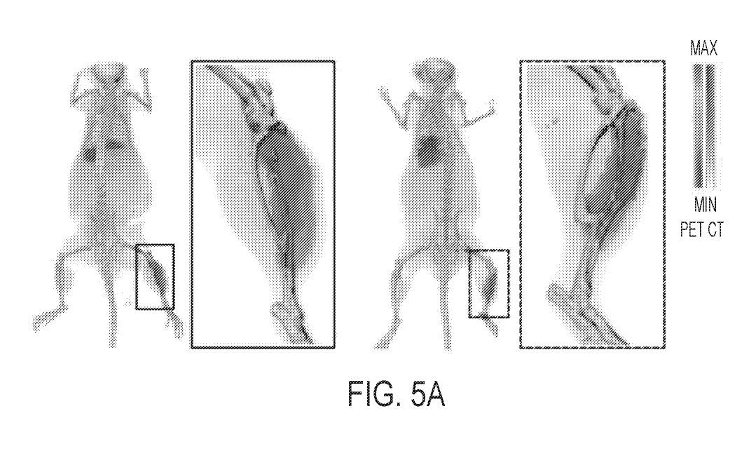

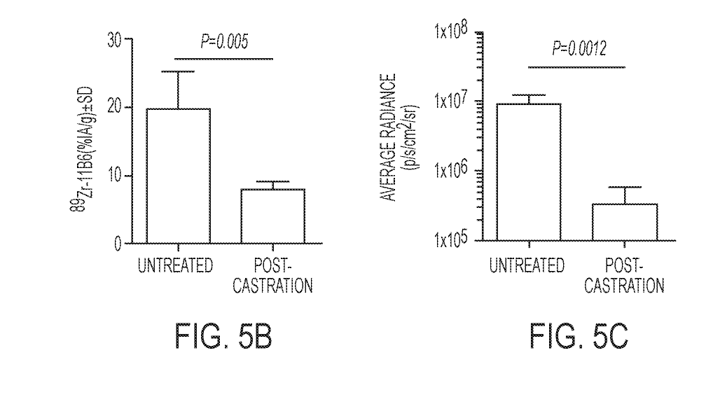

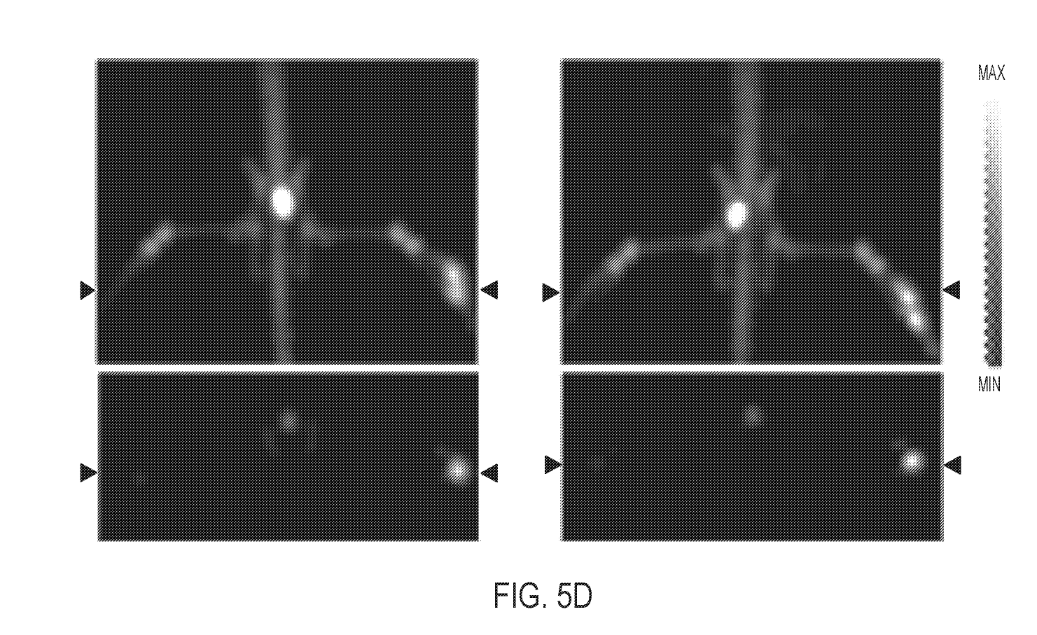

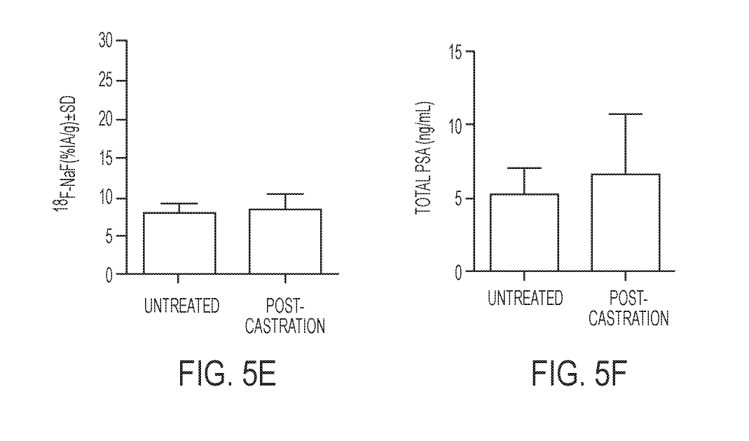

Use of the anti-hK2 tracer to assess AR-activity in response to intervention was studied in three clinical scenarios that currently lack (but would greatly benefit from) molecularly specific assessment. In the first sub-study, ⁸⁹Zr-11B6 was measured before and after surgical castration in a bone metastasis model using LNCaP-AR/luc (expressing luciferase under the control of ARR2-Pb). Standard-of-care blood measurements of PSA and ¹⁸F sodium fluoride (18 imaging. ⁸⁹F—NaF) PET bone scans were compared to hK2-targeted PET Zr-11B6 uptake decreased following castration (P=0.005; FIGS. 5A-5C), as did AR-driven luciferase (P=0.0012; FIG. 5D). Conventional metrics of prostate cancer bone lesion response, PSA and ¹⁸F—NaF, remained unchanged (FIGS. 5E-5F).

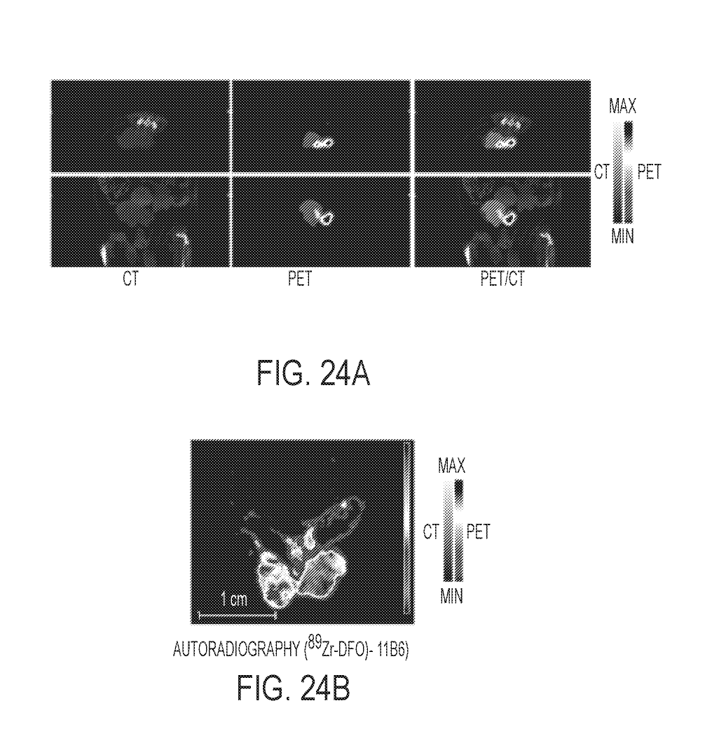

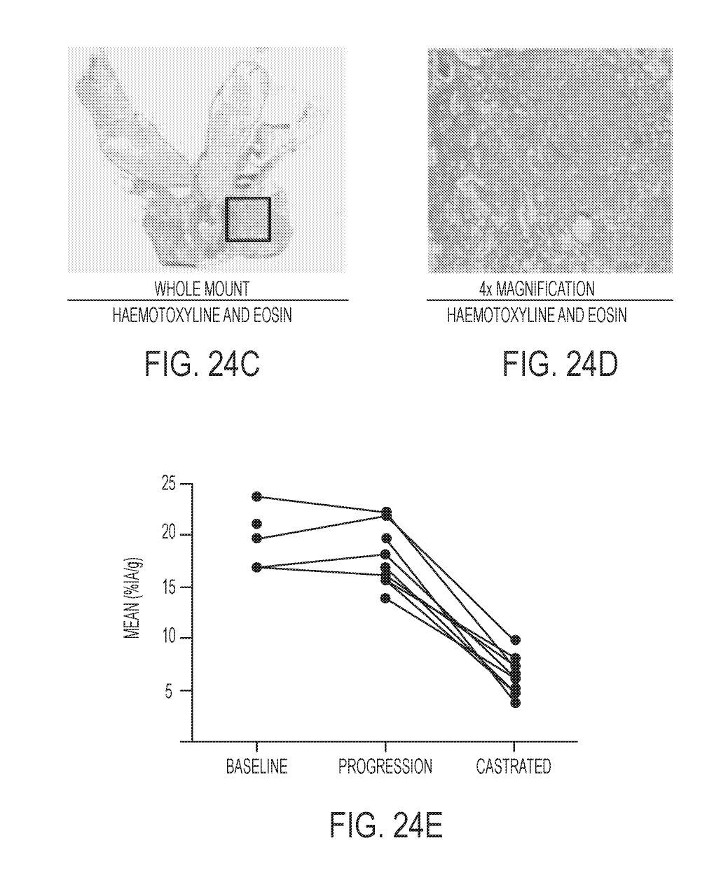

The second scenario simulated intermittent androgen deprivation (IAD) therapy. There is debate concerning the optimal treatment regime (between intermittent or continuous inhibition) for hormonal therapy. Pb_KLK2 XHi-Myc mice received depot injections of Degarelix, a gonadotrophin-releasing hormone (GnRH) antagonist, ablating androgen production for 2 months. ⁸⁹Zr-11B6 imaging was performed longitudinally to assess response to androgen deprivation, as well as reactivation following discontinuation. ⁸⁹Zr-11B6 decreased following castration but reemerged at the end of the treatment period, enabling readout of pharmacodynamic inhibition of the AR pathway (FIGS. 24A-24E).

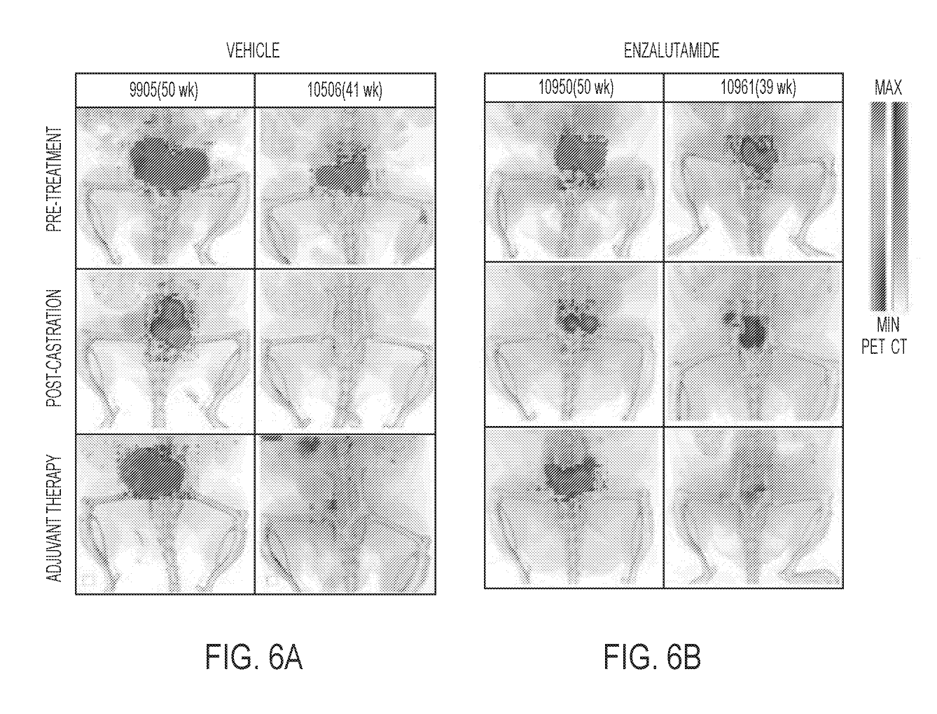

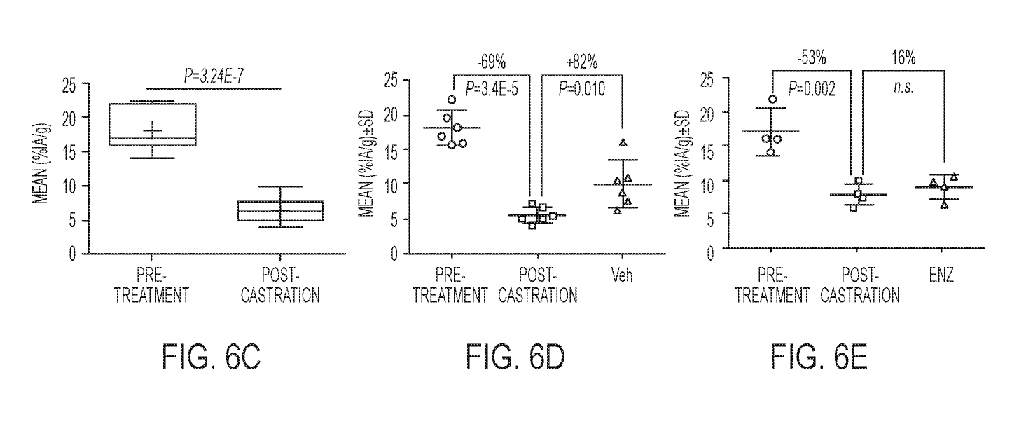

A final clinical simulation involved non-invasive imaging of the impact of different degrees of inhibition on AR activity in the tumor (intratumoral) and prostate itself (intraprostatic). Progression of disease was initially monitored in 14 Pb_KLK2 XHi-Myc mice using ⁸⁹Zr-11B6 PET. Thereafter, mice were randomized into 3 treatment groups: vehicle (n=4), castration (n=6), or castration plus Enzalutamide (n=4). Substantial heterogeneity was noted in individual subject's (e.g., animal's) prostatic uptake of the tracer during progression and in response to therapy (FIGS. 6A-6C).

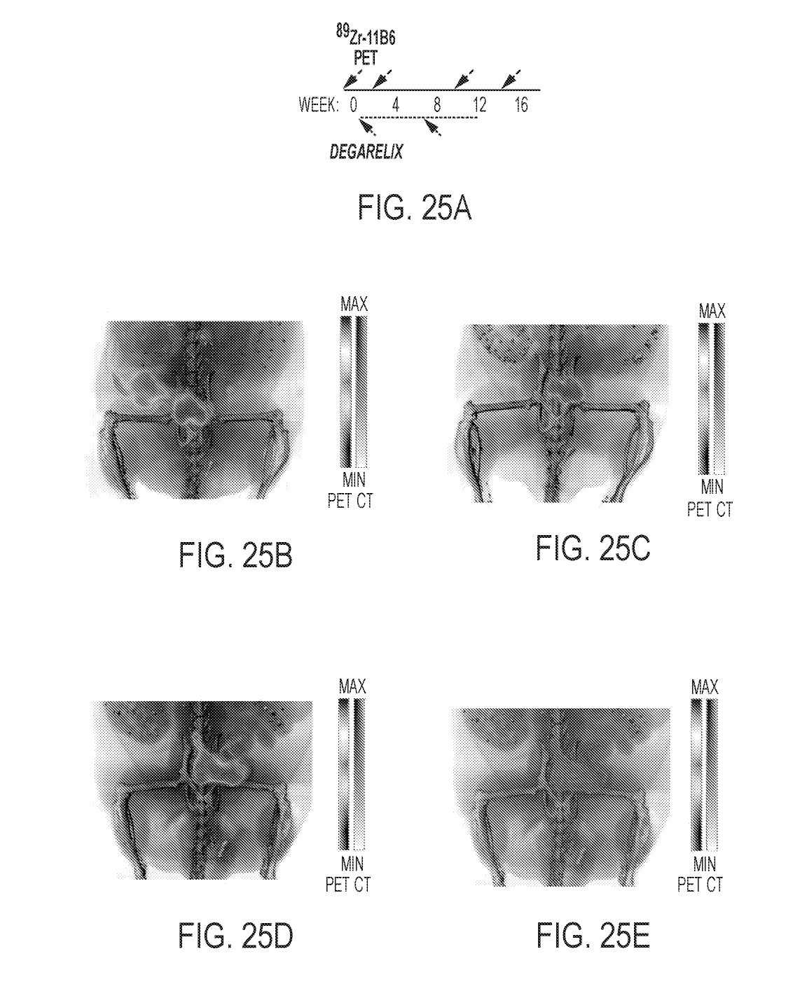

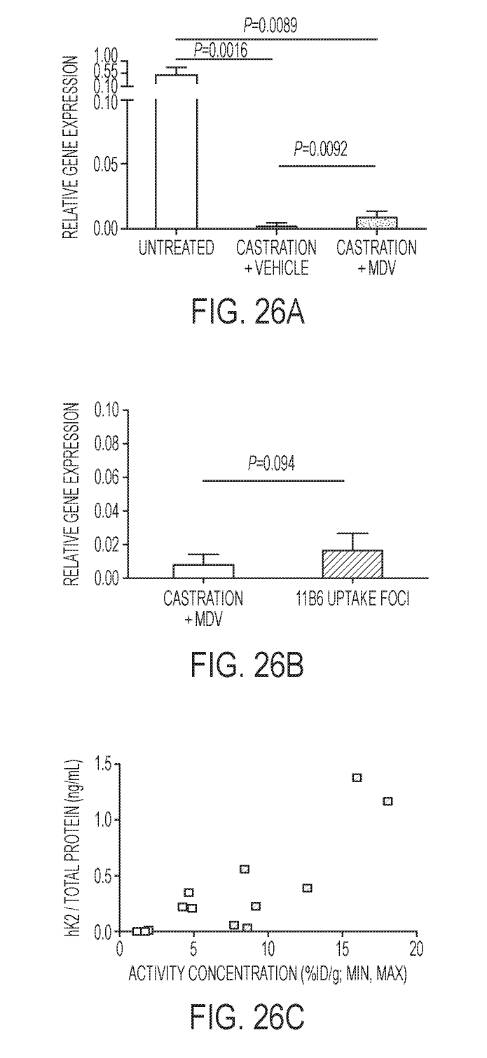

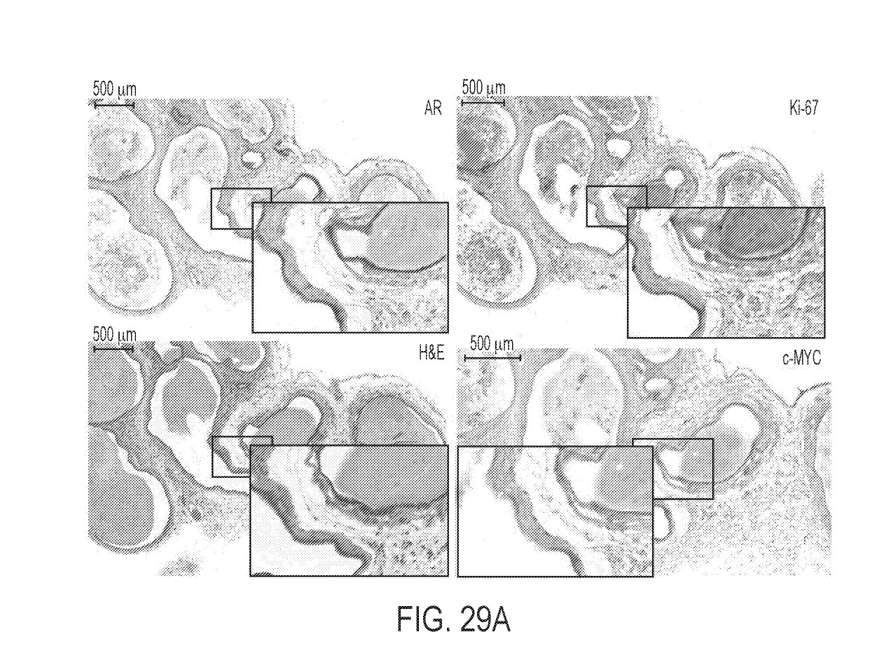

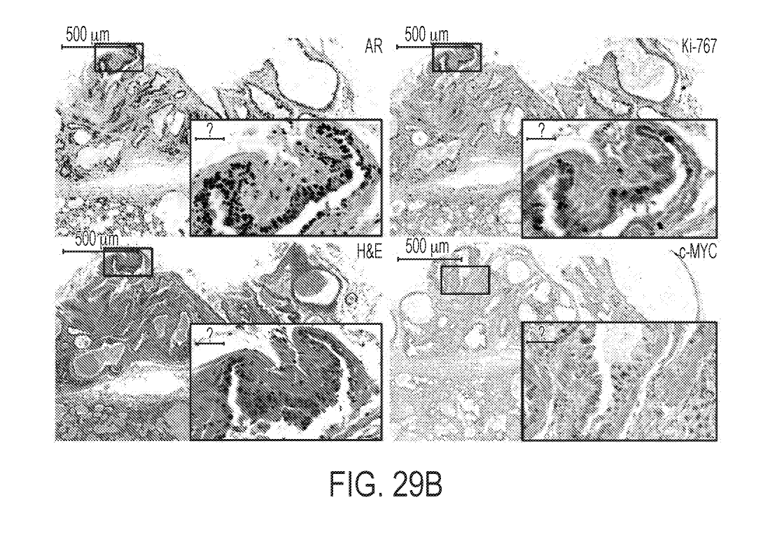

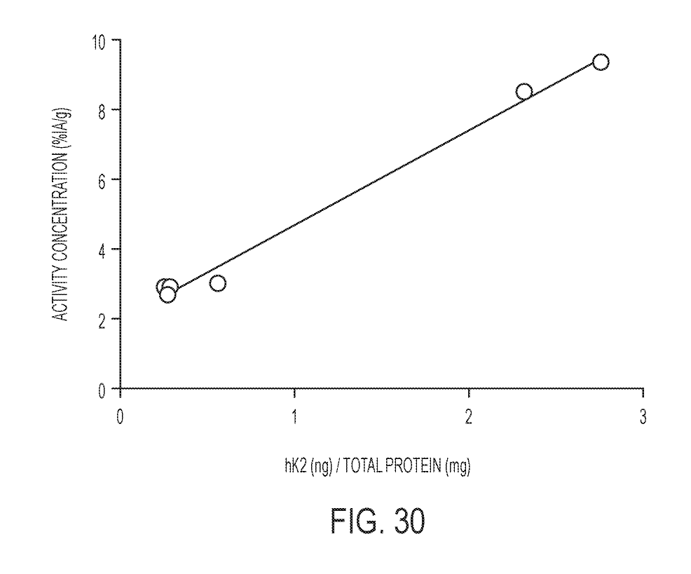

SATA uptake was repressed during the last months of treatment in mice receiving adjuvant AR blockade, indicating a benefit for adjuvant AR blockade using anti-androgens in the post-castration setting (FIGS. 6D, 6E). Reverse transcription polymerase chain reaction (RT-PCR) analysis of prostatic tissue harvested from the mice receiving AR-blockade displayed significantly lower KLK2 expression compared to other groups (P=0.0016 for castrate alone, and P=0.0089 for castration and enzalutamide; FIG. 26A). Expression differences with and without adjuvant therapy were small, as were between the lobes of the prostate containing focal sites of uptake from those that were negative (FIG. 26B). KLK2 This islikely due to the fact that RT-PCR reflects an average of the expression based on the whole lobe (FIGS. 25A-25E). However, the hK2 concentration in prostatic tissue lysate indicated a strong positive correlation between ⁸⁹Zr-11B6 uptake and AR-dependent hK2 production (FIG. 29), and immunopathology for proliferation marker Ki67 and AR (FIG. 30) reveal sub-regions that continue to proliferate following treatment which are selected by focal 11B6-signal. (FIGS. 23, 26A-26C).

### Directing Treatment in Real-Time

Radio- and fluorescently-labeled tracers indicated highly specific uptake in the cells of the prostate for noninvasive assessment (FIG. 3). To demonstrate the value of 11B6 imaging prostatic expression in the translational setting, the full treatment course was simulated to encompass pre-, intra-, and post-operative clinical decision points using dual-labeled ⁸⁹Zr-DFO and Cy5.5 for PET and fluorescence. This concept was explored using the Pb_KLK2×Hi-Myc model.

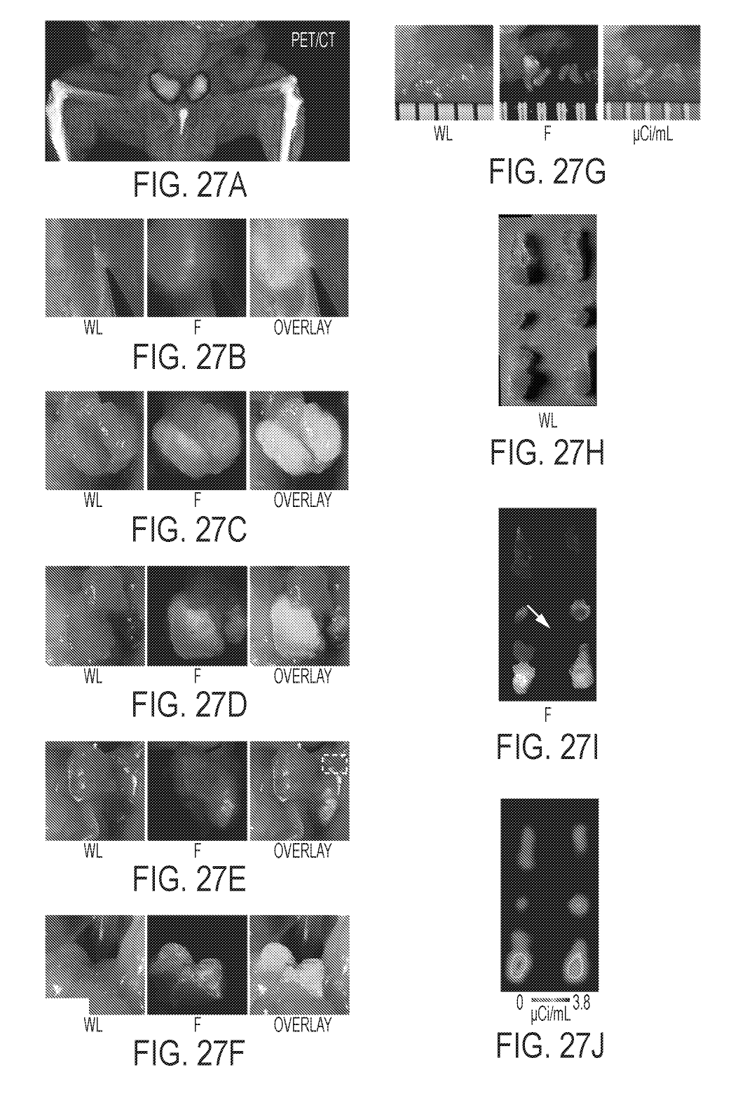

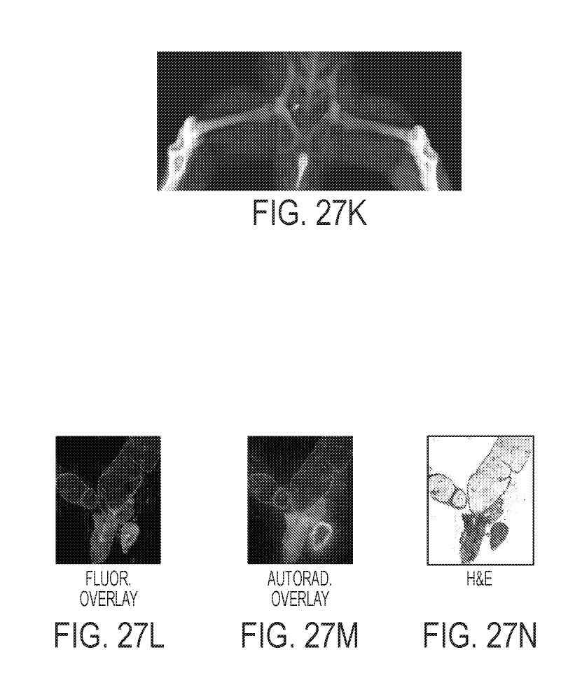

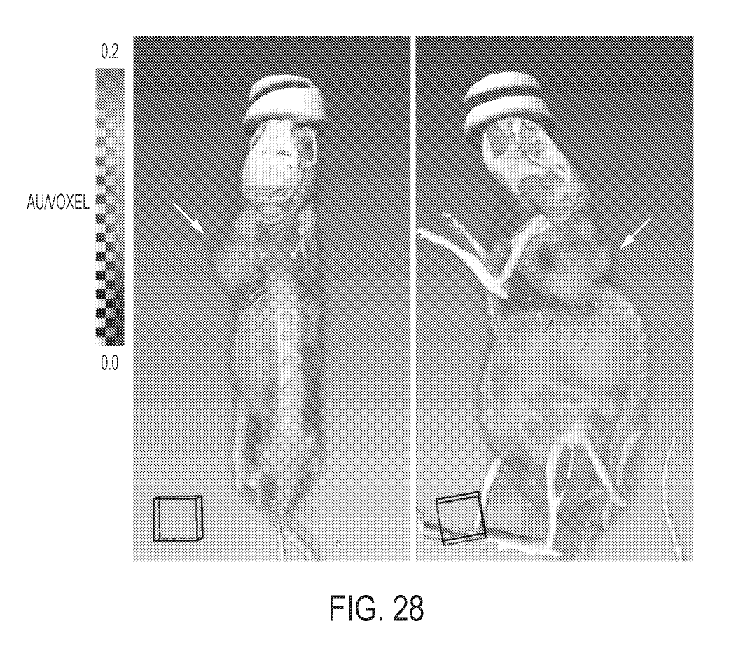

PET was performed to assess disease burden (FIG. 27A), which was then resected using a fluorescent surgical stereoscope for real-time guidance (FIGS. 27A-27G). Remnant prostatic tissue was harvested to confirm margins, and excised tissues were scanned for fluorescent and radio signals and hK2 protein (FIGS. 27H-27J). After removing fluorescent tissues, peritoneum and skin were sutured, and a post-operative PET was acquired (FIG. 27K). A region of tracer accumulation could be identified by post-operative PET/CT and was subsequently removed at autopsy. This was confirmed to be prostate tissue with fluorescence microscopy, autoradiography, and histochemistry (FIGS. 27L-27N).

### Humanized 11B6 and Toxicity

For intended use in humans, the rodent CDRs were grafted into a human immunoglobulin framework to yield hu11B6, without adverse effects on binding affinity or specificity. Surface plasmon resonance-determined dissociation and association rate constants for all versions of 11B6 were calculated to be in the range of 10-5 (koff) and 105 M−1 s−1 (kon), respectively. No statistical difference in the apparent affinity was observed between hu11B6 and its DFO conjugate (FIG. 10).

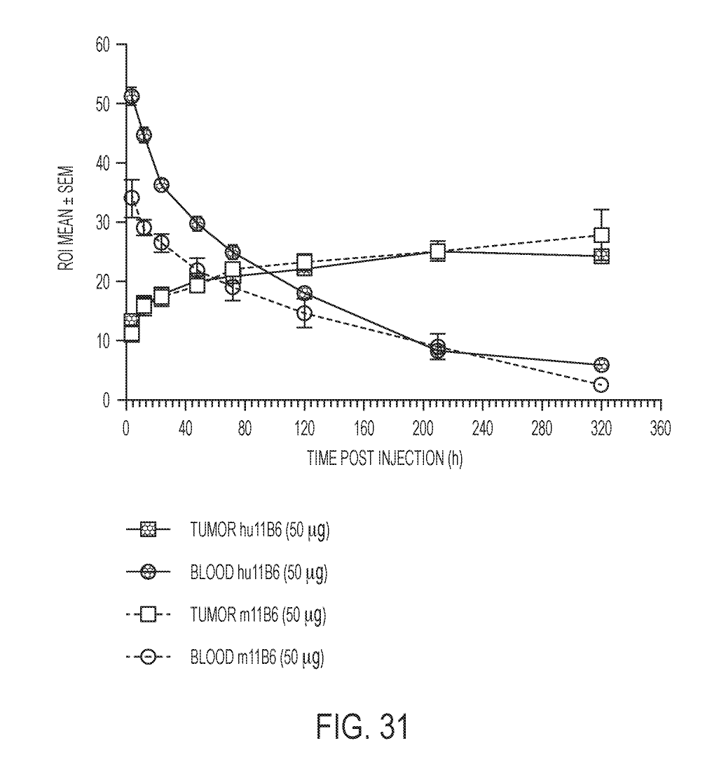

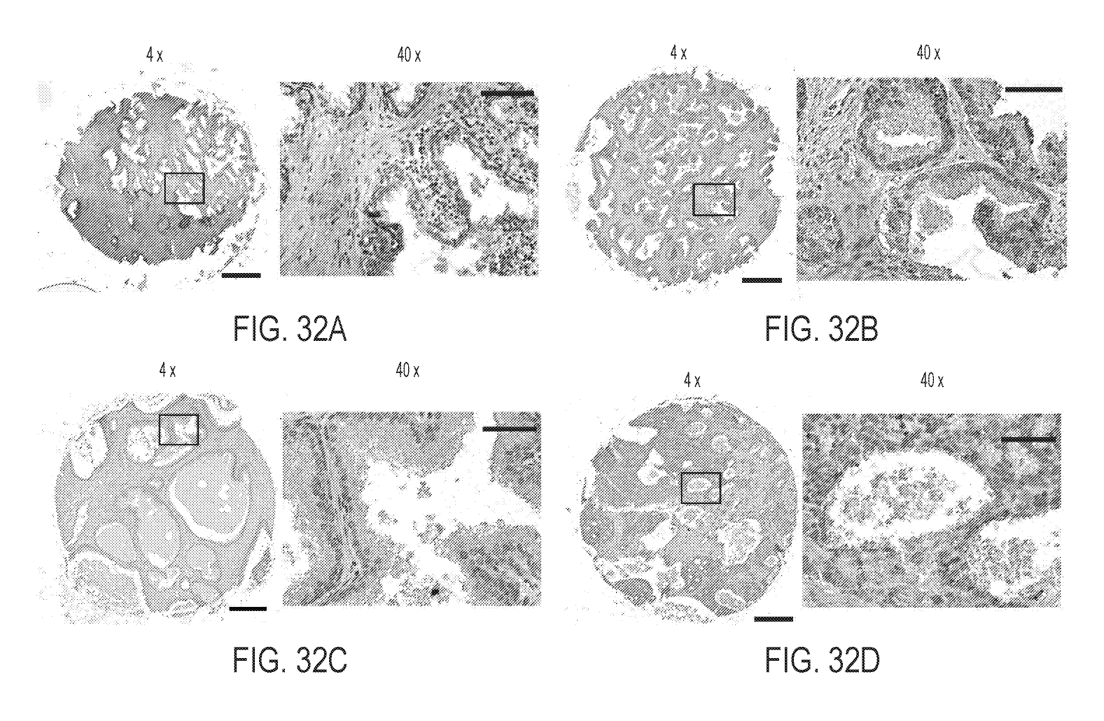

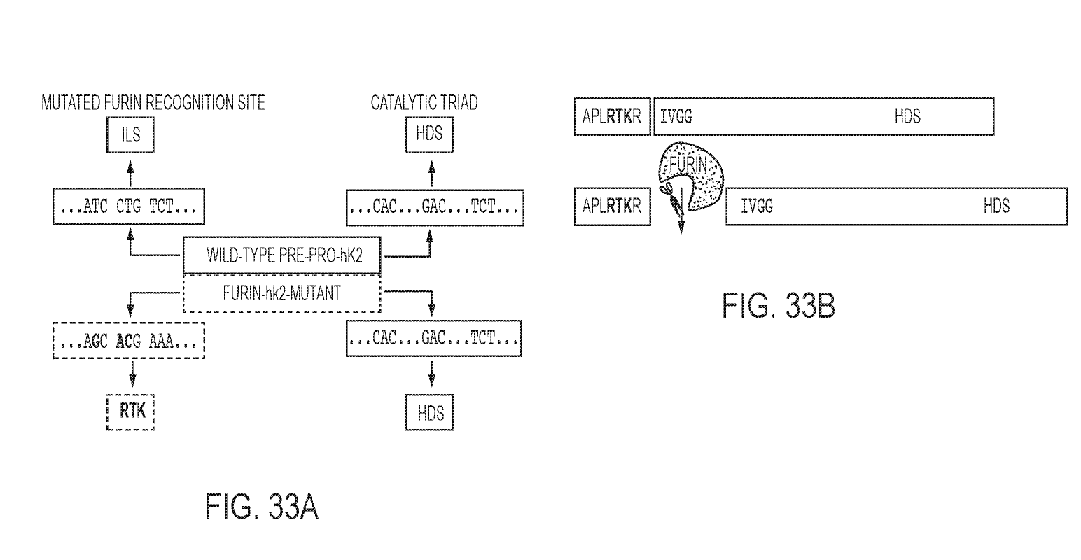

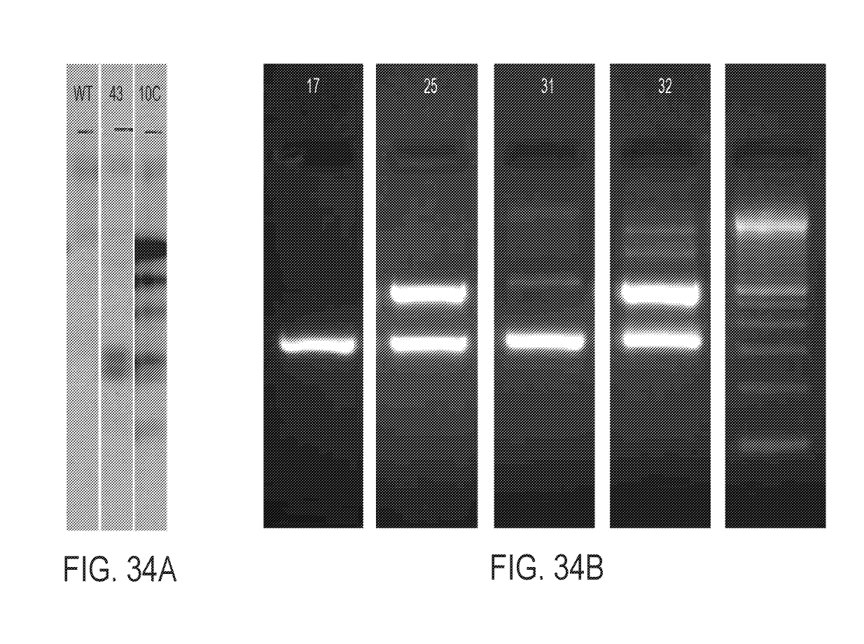

The kinetics and accumulation of the humanized conjugate, ⁸⁹Zr-DFO-hu11B6, were not significantly different from the mouse IgG1 version of 11B6 (FIG. 31). The affinity and favorable toxicity of this internalized SATA give it considerable translational potential. Finally, to assess the capacity to bind hK2 in human tissues, 11B6 was applied to human tissue specimens. The hK2 distribution in normal prostate, prostate adenocarcinoma, and a bone lesion can be identified by 11B6 immunodetection (FIGS. 32A-32D).

### Discussion

The ability to detect malignant cells, to monitor pathological processes, or to deliver therapeutic compounds is needed to improve PCa and TN-BCa management. Extracellular cytokines and proteins are recognized as important mediators of these diseases, and have been widely targeted with antibodies to combat disease or ameliorate its symptoms. However, biologics directed to these extracellular components have not enabled cellular targeting for imaging or treatment, limiting the ability to affect diseased cells themselves. Here, it is reported that an anti-hK2 antibody, 11B6, enables cell-specific accumulation of diagnostic and therapeutic agents to the most common invasive cancers in men and women.

Uptake of 11B6 in hK2-expressing tissues was FcRn-mediated, which is a unique demonstration of antibody-antigen internalization by cells which themselves express the target. FcRn enables passive transfer of IgG from mother to offspring in the early stages of life as well as a variety of physiologic functions in adult immunity. Notably, FcRn facilitates transport of IgG₁ and recycling of IgG-immune complexes across otherwise impermeable polarized epithelia. 11B6 exploits this mechanism, resulting in cellular accumulation of an immune complex which avoids the precipitous washout observed using a previous kallikrein-targeted construct. The wider applicability of this approach to enable cell specific accumulation of a second SATA to PSA (5A10^{H435-wt}) is demonstrated herein. FcRn binding is pH dependent and the lower pH at sites of disease may provide an even more favorable microenvironment to generate imaging contrast compared to non-malignant tissue. These results have immediate relevance for both PCa and BCa directed imaging and therapy and more widely as a strategy to improve both the magnitude and localization of internalizing SATA.

hK2 has traditionally been evaluated as a prostate biomarker; however, shown herein is uptake of ⁸⁹Zr-11B6 in AR-positive breast cancer xenografts under hormone stimulation. Questions surround the repercussions of AR status in BCa. While several studies implicate a role for AR in pathways that negatively impact survival, a correlation between AR and positive prognostic markers has also been identified. The application of androgen antagonists in AR-positive BCa indicates that AR inhibition may be best directed towards basal (triple-negative) rather than luminal B type/HER2 refractory subtypes. Without wishing to be bound to any theory, trials suggest that this may represent a new approach to treat TN-BCa. The 11B6 platform enables further study of the nuanced role of AR in the biology of breast cancer by offering the ability to guide and monitor treatment.

The multimodal methods employed against a range of models demonstrate an approach which eliminates long-standing impediments to non-invasively monitor disease biology and assist development of novel androgen receptor-targeted therapies as a pharmacodynamic tool. Biopsy is used in PCa and BCa disease assessment to provide direct readout of tissue organization but is restricted in time, access and accuracy. Conventional imaging to guide biopsy (ultrasound, computed tomography (CT) and MRI) suffers from modest sensitivities for detection and staging, with complication risks. If lesions are detectable, a direct biopsy can provide information on cellular processes, but is invasive, costly and difficult to repeat.

In contrast, ⁸⁹Zr-11B6 PET provides whole-body imaging of disease foci and provides a readout of AR activity for both primary and metastatic lesions. In a transgenic c-Myc driven model of adenocarcinoma, AR-activity was longitudinally evaluated during disease progression from the pre-malignant prostate through high disease burden (FIG. 4). The dynamics of androgen inhibition, for example with metronomic chemical castration, can be monitored quantitatively. This imaging platform can be extended to evaluate treatment regimens, which revealed low levels of AR-pathway reactivation at sub-organ resolution and enabled a comparison between models of surgical castration versus castration plus adjuvant therapy (FIGS. 6A-6E). The agent may also be used to guide treatment in real-time or assist in treatment delivery.

⁸⁹Zr-11B6 targets tumorous lesions themselves, rather than sites of remodeling, and is able to identify both osteoblastic and osteoclastic metastases (FIGS. 2A-2C). Conventional ¹⁸F—NaF bone scans have high sensitivity but lack specificity for disease, confounding the readout of disease burden especially post-therapy. The enhanced precision of treatment monitoring by SATA will help to accelerate preclinical and translational research towards answering critical clinical questions for optimal patient care.

The technology presented here has direct application in PCa and BCa patients. Humanized-11B6 retains binding characteristics of the original agent (FIGS. 25A-25E). The technology is applicable to individualized patient stratification and management at the molecular level. The approach of designing SATA which facilitate cellular uptake may be relevant to the detection, monitoring, and treatment of a wide variety of diseases and conditions.

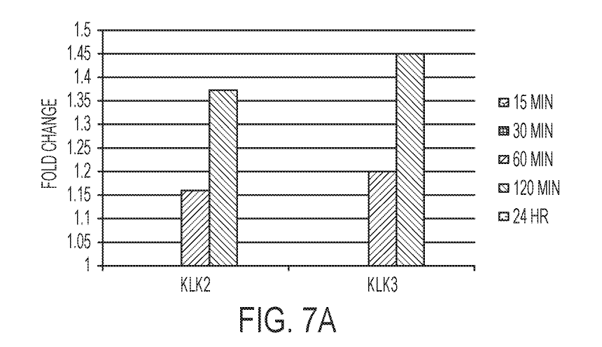

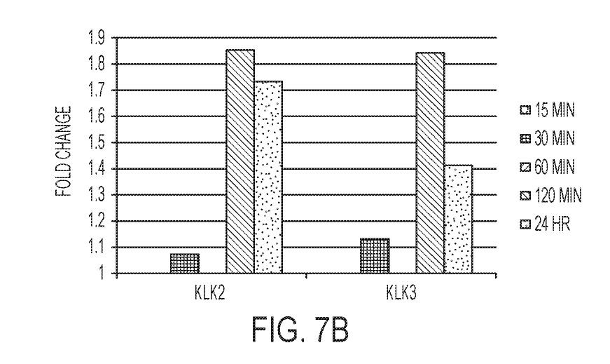

FIGS. 7A-7B present graphs that show an AR increase after irradiation of two AR-positive BCa cell lines (BT474 and MFM223). The change in KLK2 and KLK3 is shown for both BT474 and MFM following irradiation.

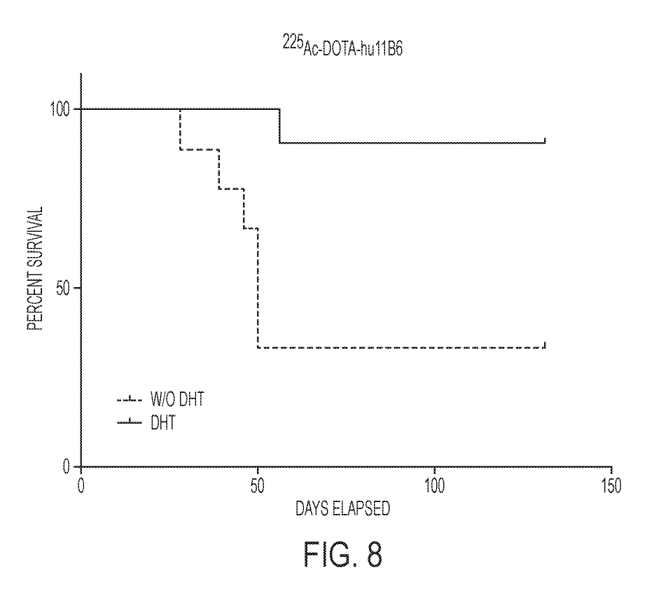

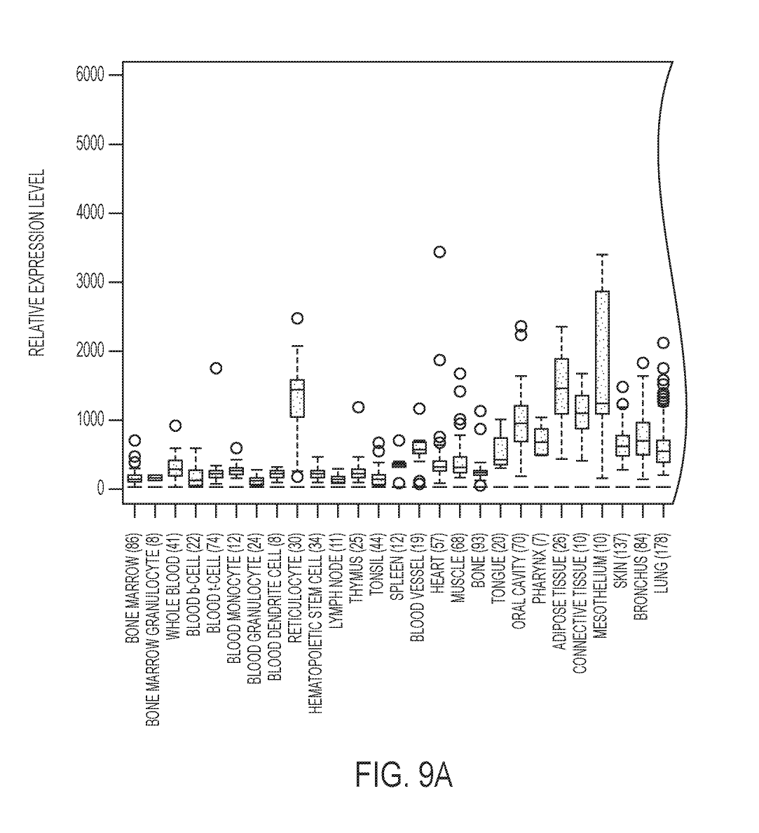

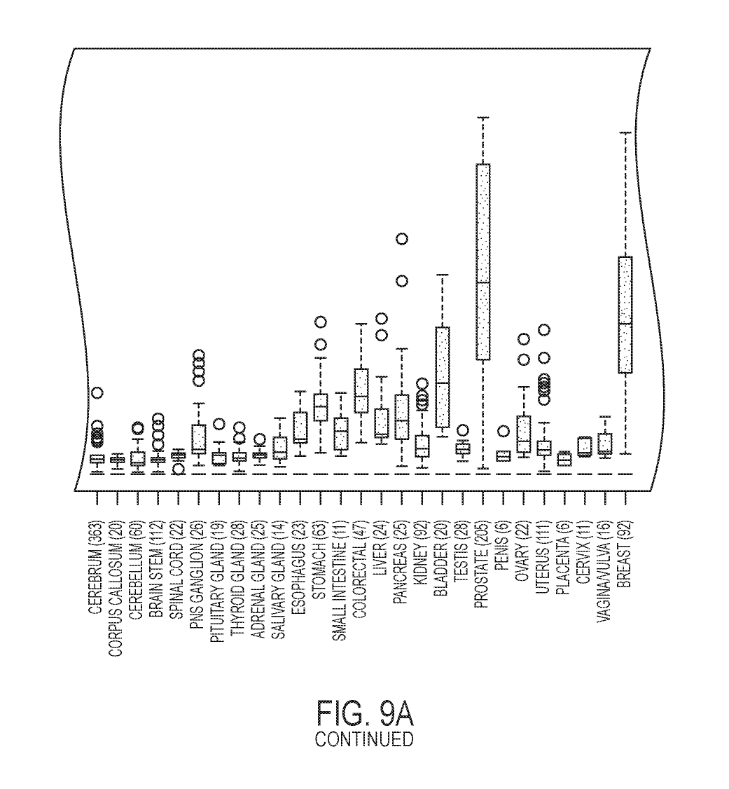

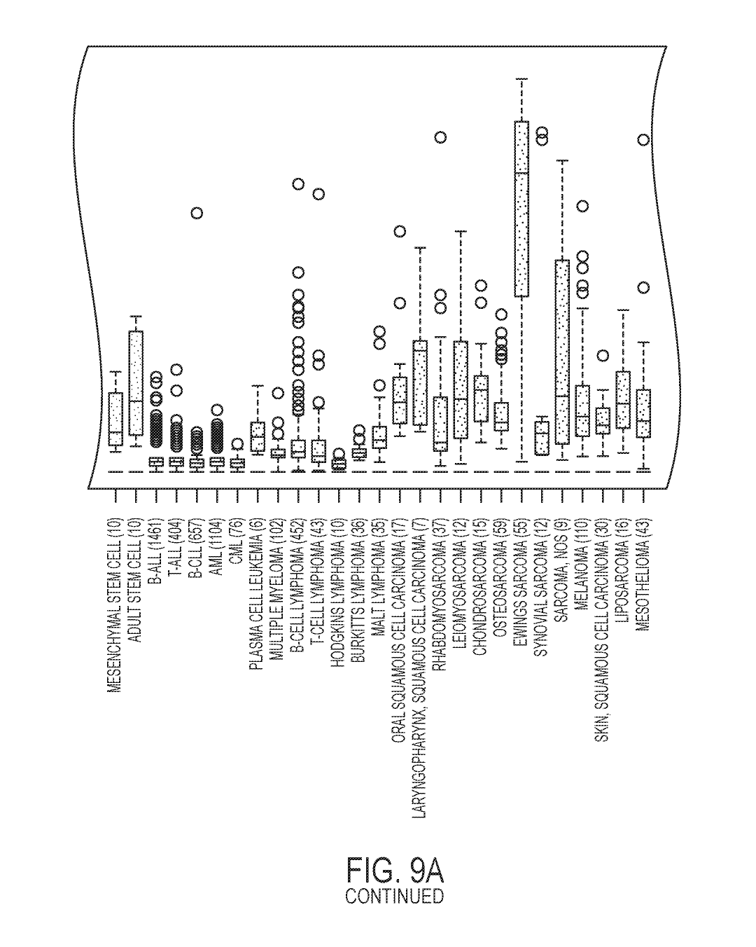

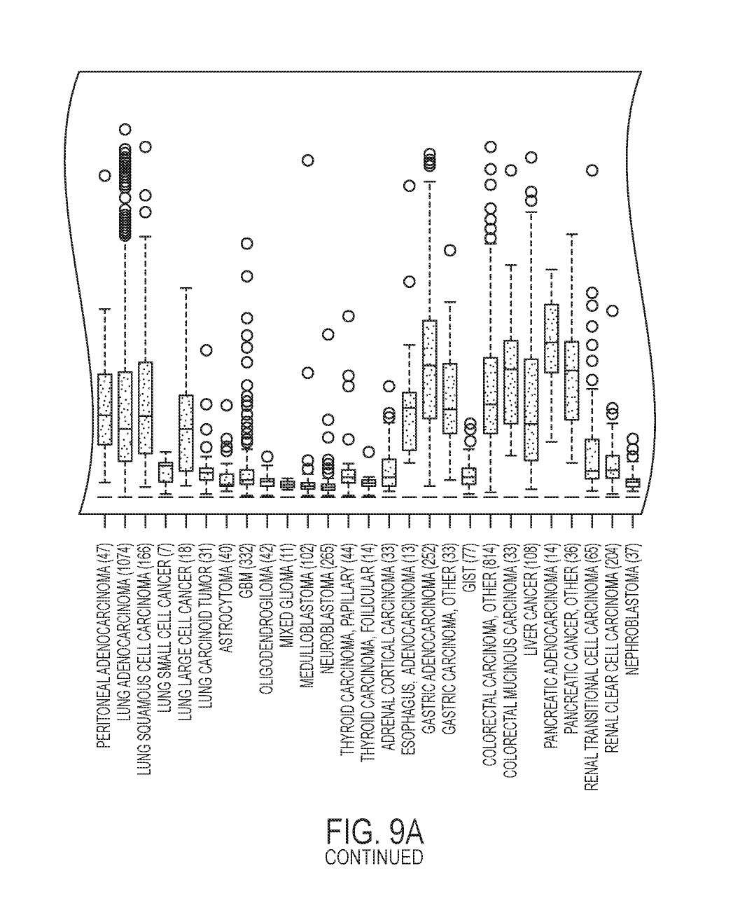

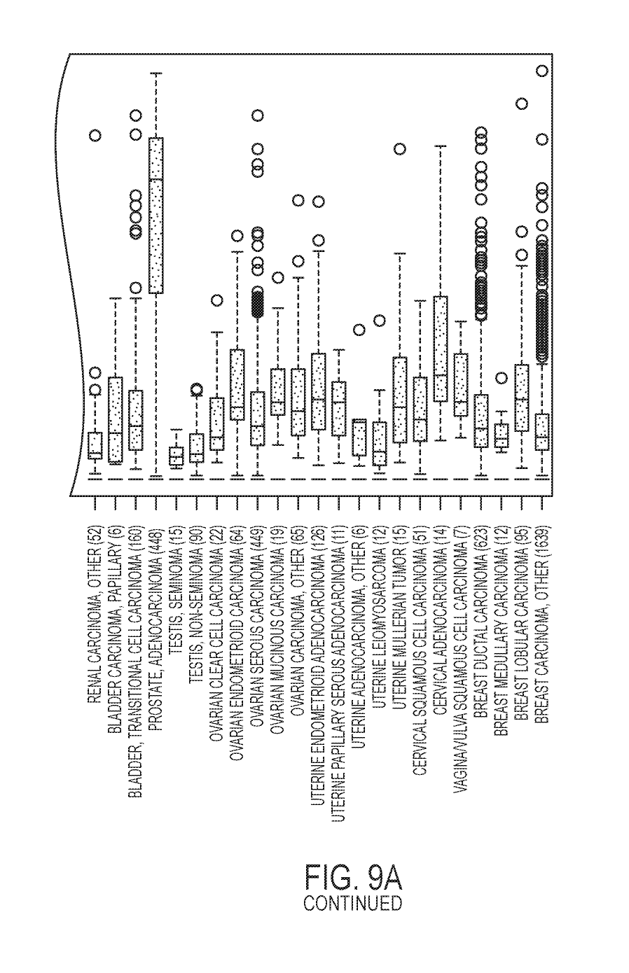

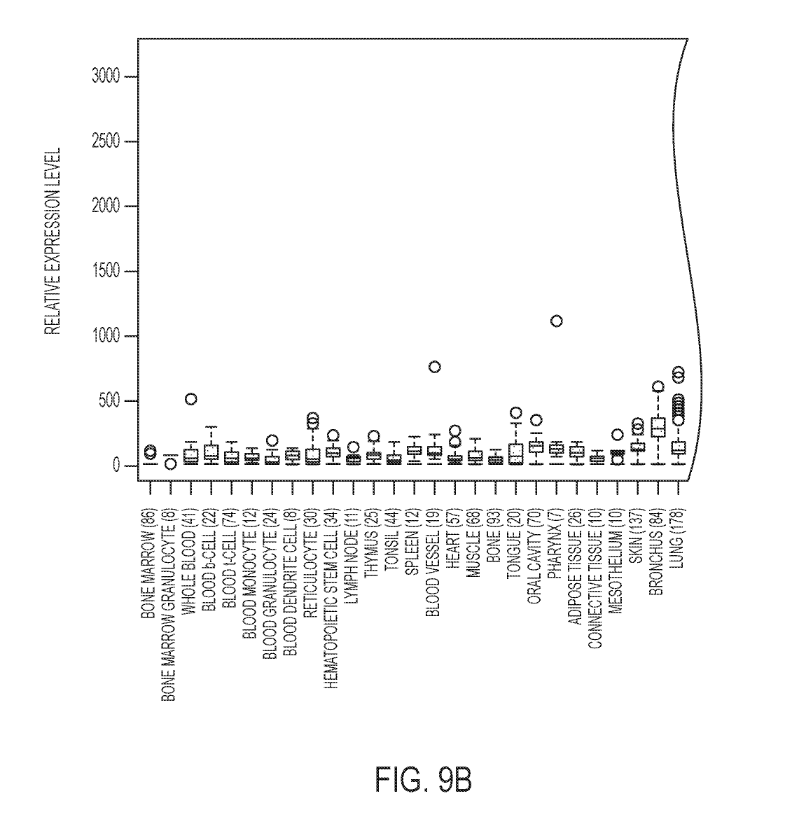

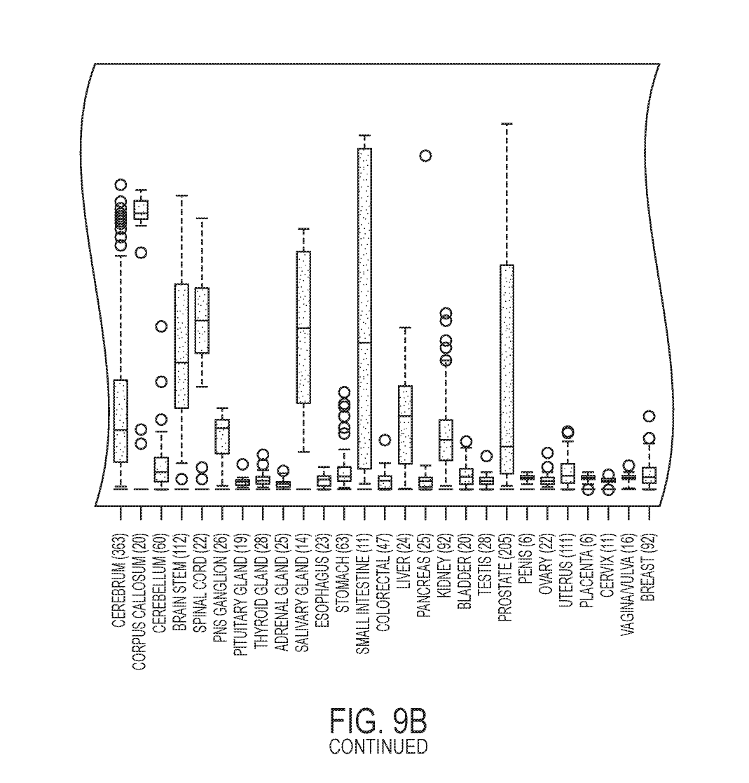

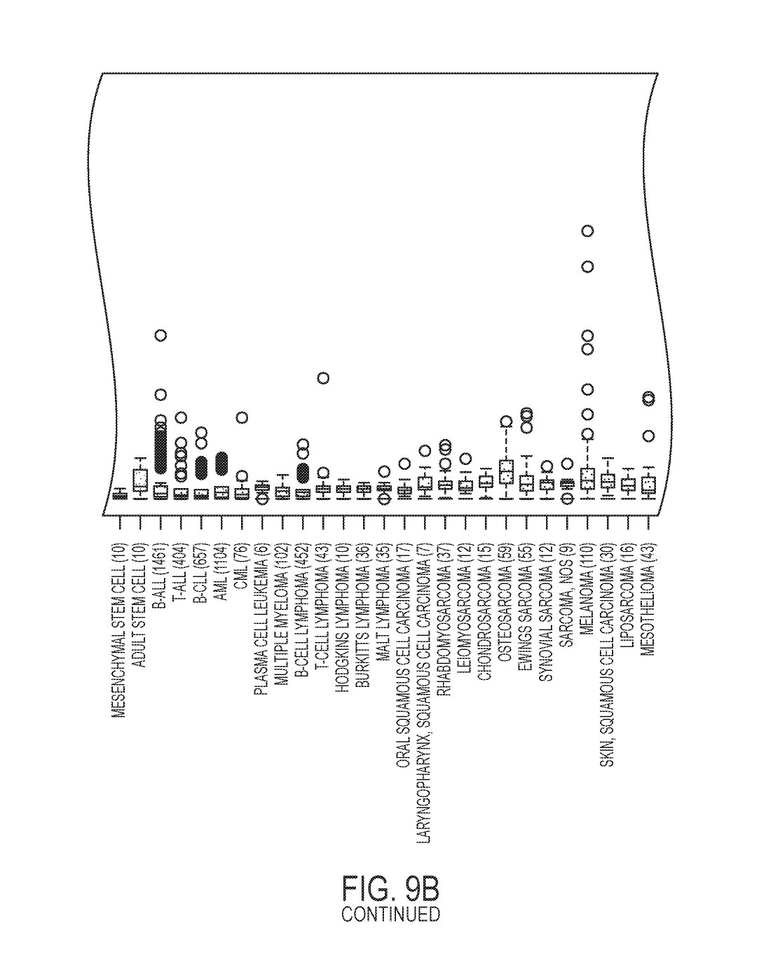

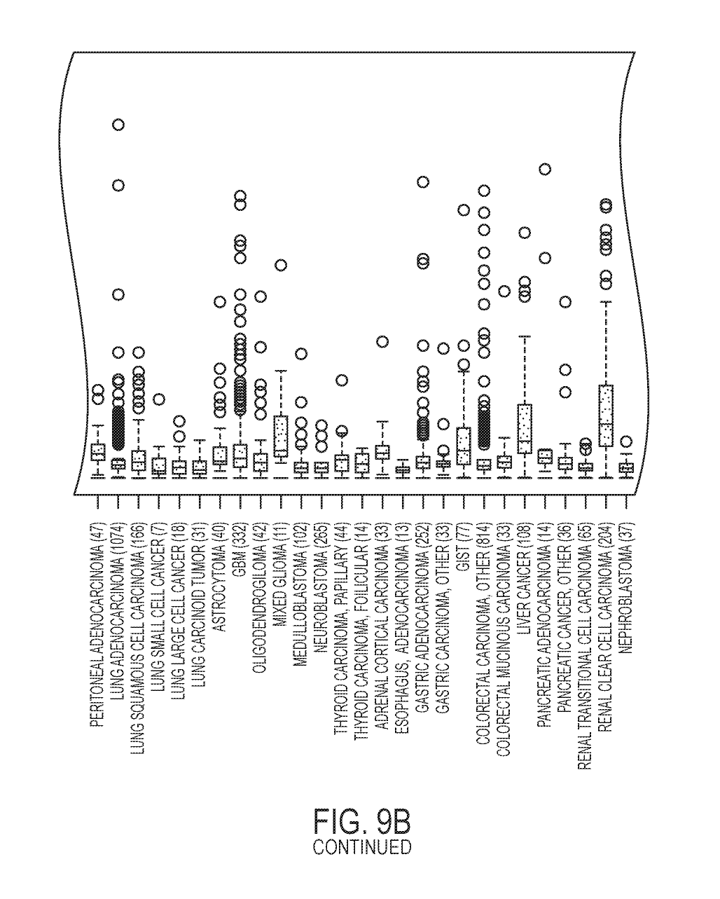

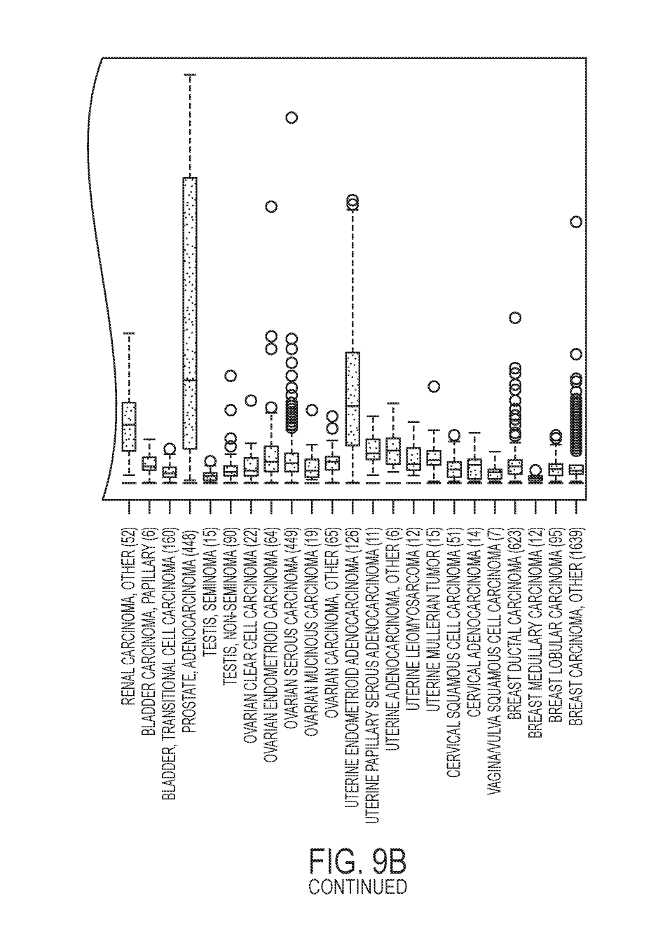

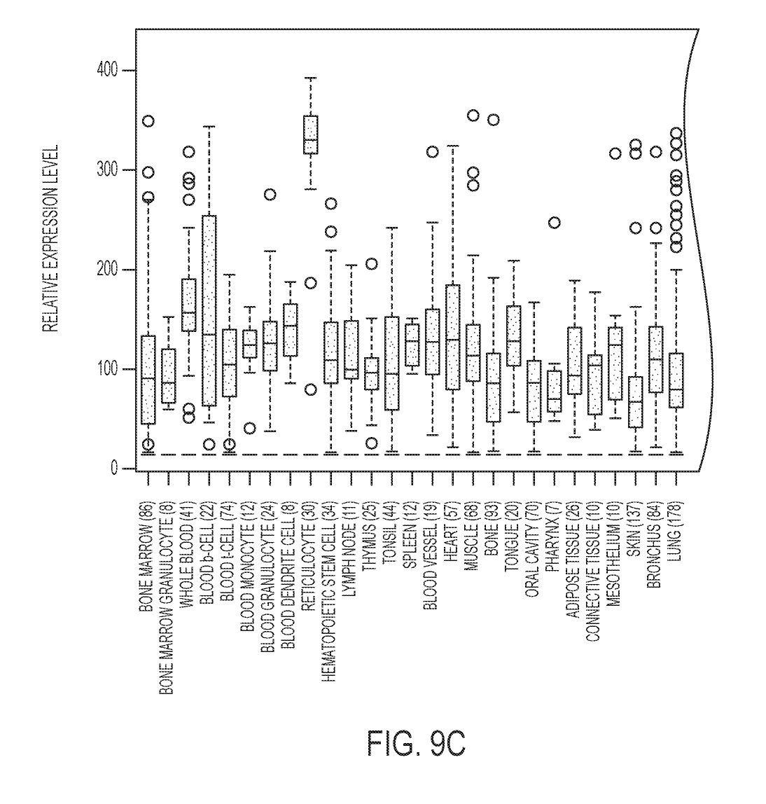

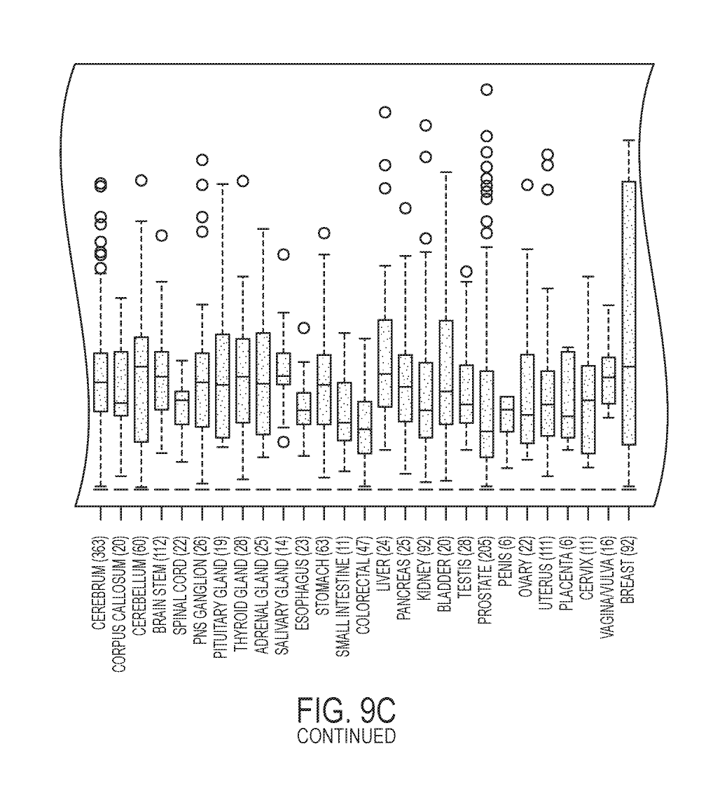

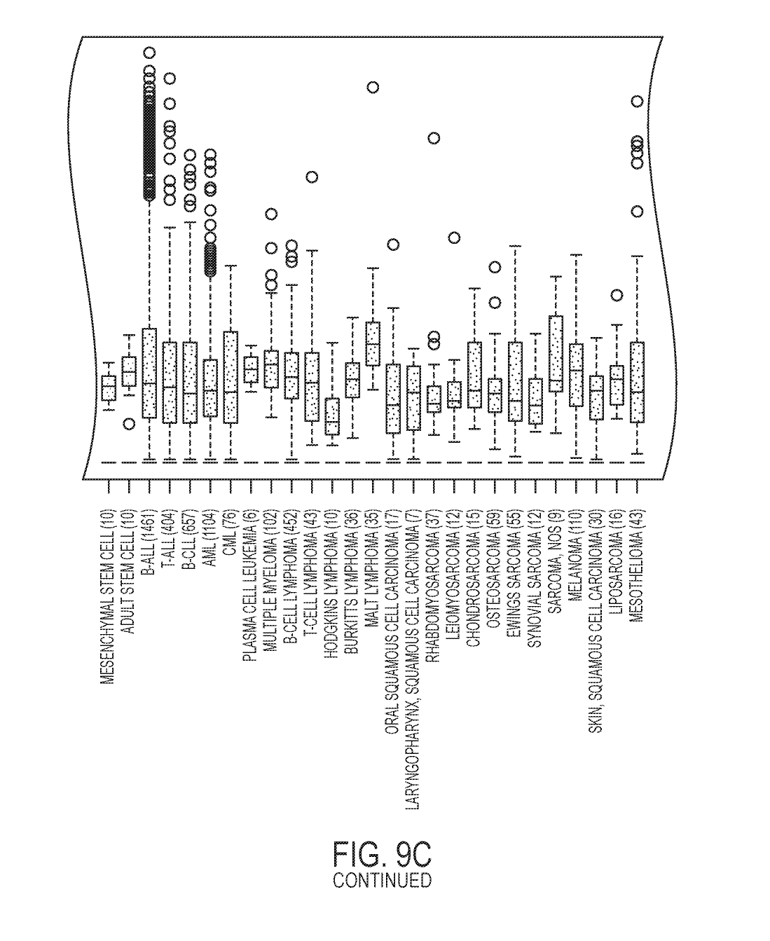

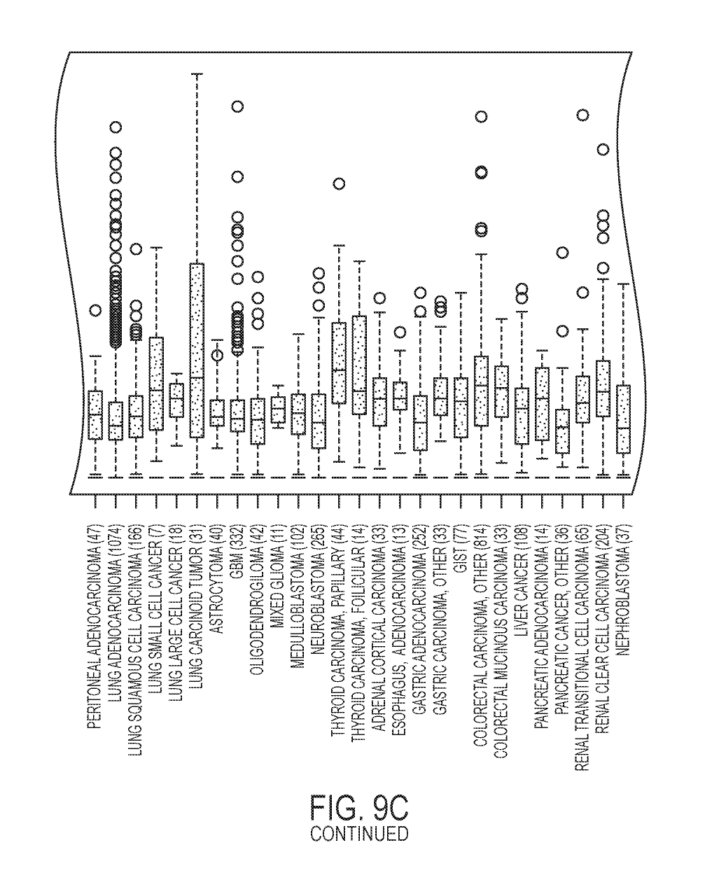

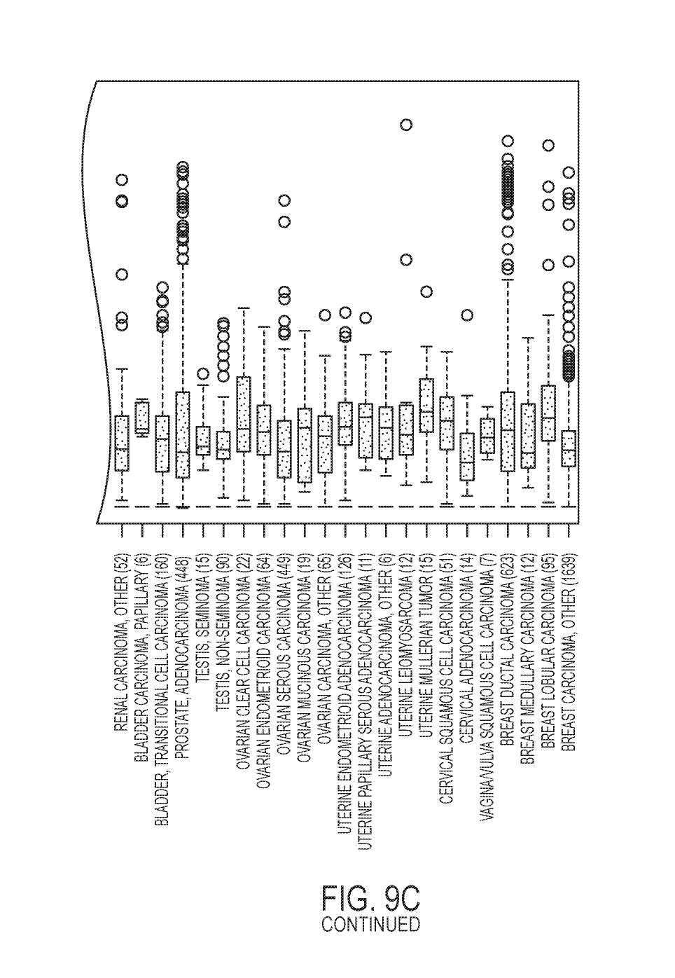

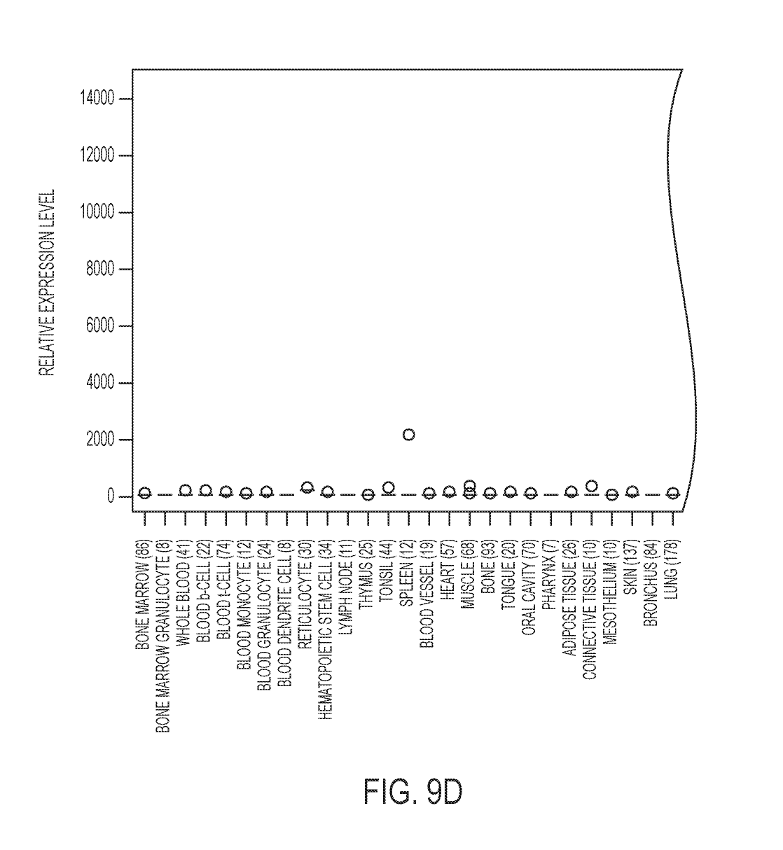

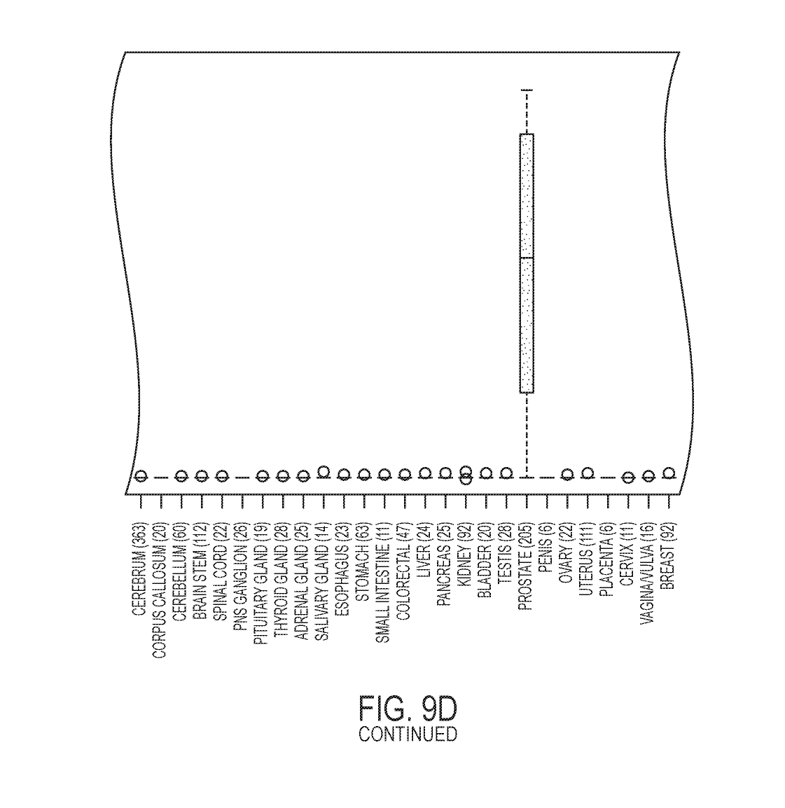

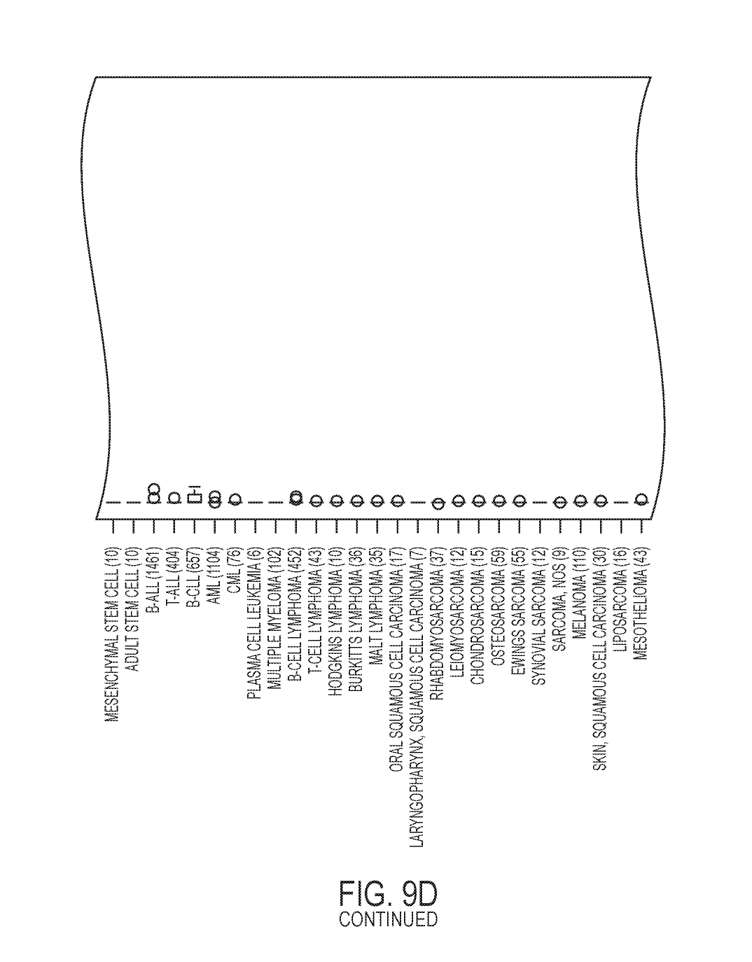

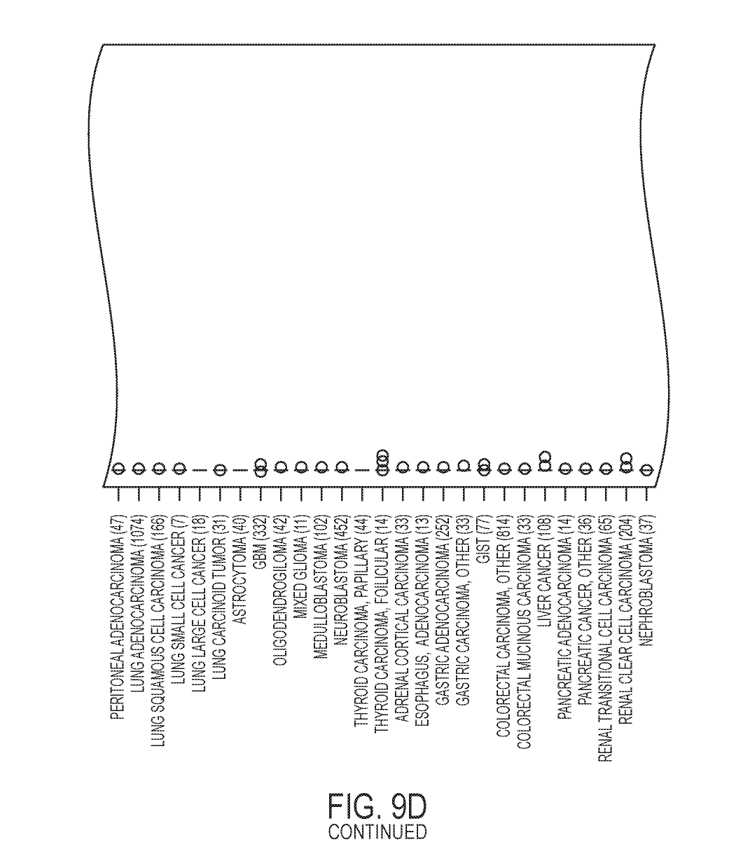

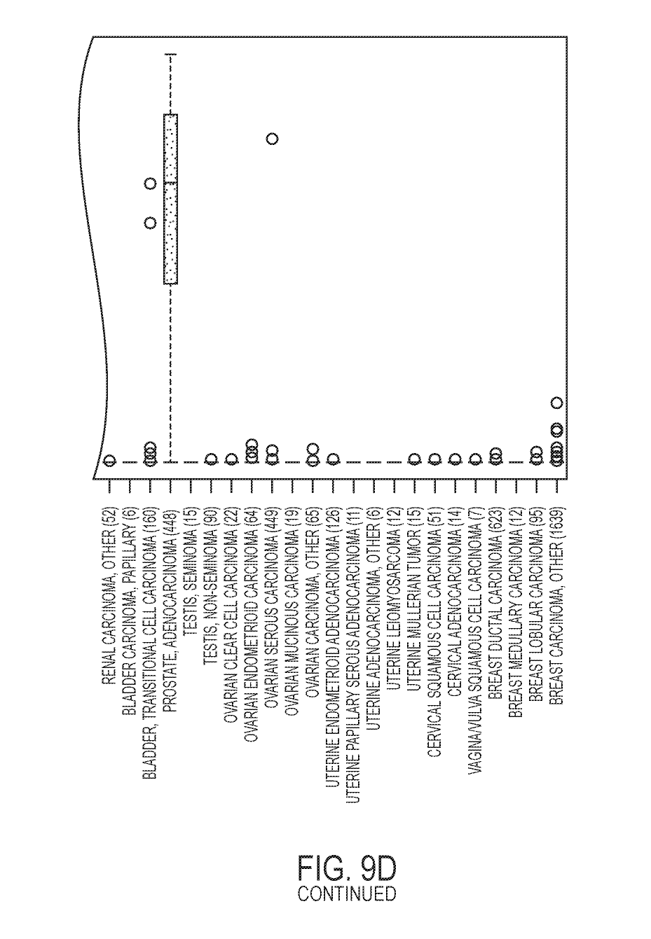

FIG. 8 shows a survival graph after injecting ²²⁵Ac-DOTA-hu11B6 in DHT-stimulated (i.e. Expression of KLK2) and in non-DHT stimulated mice (i.e. Non-KLK2 expression). FIGS. 7A-7B and FIG. 8 demonstrate the value of KLK2 and KLK3 induction—e.g., by administration of progesterone, testosterone, and/or, as shown here, by irradiation—prior to administration of a free-PSA and/or free hK2 antibody labelled with a radioisotope for radio-immunotherapy (RIT) of AR-positive breast cancer, in accordance with an illustrative embodiment of the invention.

Table 1 shows dissociation rate constants (k_{off}) for m11B6, hu11B6, and DFO-conjugated hu11B6. Based on the two measurement series taken for each antibody, no significant difference in the dissociation rate constants (k_{off}) was found between the hK2 targeting antibodies.

TABLE 1

| Antibody | k_{off} (10⁻⁵s⁻¹)Fc2 | k_{off} (10⁻⁵s⁻¹)Fc3 | k_{off} (10⁻⁵s⁻¹)Fc4 | Mean | Std dev |
|---|---|---|---|---|---|
| m11B6 | 1.9 | 4.9 | — | 3.4 | ±2.1 |
| hu11B6 | 6.4 | 6.9 | — | 6.7 | ±0.4 |
| hu11B6-DFO | 5.8 | 5.5 | — | 5.7 | ±0.2 |

Table 2 shows average association rate constant based on 15-18 measurements for each version of 11B6. Differences in rate constants (kₒₙ) of the tested antibodies were not significant.

TABLE 2

| | No. Of expts | Mean kₒₙ | |
|---|---|---|---|
| Antibody | fitted | (10⁵M⁻¹s⁻¹) | Std dev |
| m11B6 | 18/18 | 2.48 | ±0.85 |
| hu11B6 | 15/18 | 1.17 | ±0.38 |
| hu11B6-DFO | 18/18 | 1.11 | ±0.22 |

Table 3 shows dissociation rate constants (K_{D}) for the tested antibodies.

TABLE 3

| Antibody | Mean K_{D} 10⁻¹¹ M | Std dev |
|---|---|---|
| m11B6 | 19 | ±15 |
| hu11B6 | 65 | ±25 |
| hu11B6-DFO | 54 | ±13 |

Table 4 shows ⁸⁹Zr-11B6 biodistribution and the effect of blocking with cold antibody. Biodistribution values for each organ are shown as percent injected activity per gram at 320 h for different cell lines. Data are shown as mean±standard deviation with n≥3.

TABLE 4

| | | | | | | | Blocked | |
|---|---|---|---|---|---|---|---|---|
| | LNCaP | | VCaP | | DU145 | | (LNCaP) | |
| Organ | Avg. | ± | Avg. | ± | Avg. | ± | Avg. | ± |
| Blood | 3.05 | 0.61 | 2.28 | 1.11 | 6.48 | 1.90 | 4.01 | 1.25 |
| Tumor | 24.72 | 2.41 | 80.68 | 15.34 | 1.25 | 0.58 | 6.42 | 2.91 |
| Heart | 1.60 | 0.19 | 0.98 | 0.20 | 2.47 | 0.26 | 2.46 | 0.63 |
| Lung | 3.60 | 0.50 | 2.08 | 0.37 | 5.16 | 0.66 | 3.81 | 1.74 |
| Liver | 13.80 | 2.47 | 11.64 | 3.97 | 16.77 | 1.01 | 12.82 | 0.37 |
| Spleen | 6.56 | 2.86 | 8.58 | 2.00 | 4.39 | 0.33 | 6.33 | 1.08 |
| Stomach | 0.29 | 0.10 | 0.31 | 0.22 | 0.86 | 0.35 | 0.27 | 0.08 |
| Sm. Intest. | 0.52 | 0.07 | 0.48 | 0.14 | 3.37 | 4.45 | 0.41 | 0.07 |
| Lg. Intest. | 0.42 | 0.05 | 0.51 | 0.17 | 0.81 | 0.38 | 0.36 | 0.10 |
| Kidneys | 4.10 | 0.08 | 1.96 | 0.37 | 5.00 | 0.35 | 4.59 | 1.23 |
| Muscle | 0.38 | 0.16 | 0.44 | 0.12 | 0.60 | 0.13 | 0.57 | 0.24 |
| Bone | 5.52 | 2.18 | 1.40 | 0.56 | 1.23 | 0.32 | 6.33 | 3.42 |

Table 5 shows receptor status of breast cancer cell lines and secretion of hK2 in response to DHT. The status of estrogen and progesterone receptor and HER2 amplification, as well as the presence of AR for common breast cancer cell lines are given. These 13 BCa cell lines were tested by immunofluorimetric assay for the presence of hK2 protein secretion in culture supernatant. No cells produced the kallikrein without hormone stimulation, and only AR-positive cell lines were found to produce hK2 after the addition of the hormone.

TABLE 5

| | Hormone status | | | | hK2 |
|---|---|---|---|---|---|
| Human | | | | Andro- | produced |
| breast cancer | Estrogen | Progesterone | | gen | after AR/PR |
| line | receptor | receptor | HER2 | receptor | stimulation |
| AU565 | − | − | + | − | − |
| BT-20 | − | − | − | − | − |
| BT-474 | + | + | + | + | + |
| HCC1806 | − | − | − | − | − |
| MCF7 | + | + | − | − | − |
| MDAMB361 | + | + | + | − | − |
| MDAMB415 | + | − | − | − | − |
| MDAMB435 | − | − | − | − | − |
| MDAMB468 | − | − | − | − | − |
| MFM-223 | − | − | − | + | + |
| SK-BR-3 | − | − | + | − | − |
| T-47D | + | + | − | + | + |
| ZR-75-30 | + | − | + | − | − |

Table 6 shows data values from PET, bioluminescence and clinical chemistry measurements. The data is shown for each group pre- and post-castration, appended with average and standard deviation computations. Insufficient bloods for two animals in the PSA assay reduce the group size for this measurement to n=4.

TABLE 6

| | Measurement (units) | | | | | | | |
|---|---|---|---|---|---|---|---|---|
| | | | | | | | Average | |
| | ⁸⁹Zr-11B6 | | Radiance | | Total PSA | | 18F-NaF | |
| | (mean % IA/g) | | (p/s/cm2/sr) | | (ng/mL) | | (mean % IA/g) | |
| | | Post- | | Post- | | Post- | | Post- |
| | Untreated | Castration | Untreated | Castration | Untreated | Castration | Untreated | Castration |
| | 14.64782 | 6.861449 | 10640000 | 86260 | | 1.1595 | 7.26063 | 7.077255 |
| | 25.77738 | 8.707577 | 11030000 | 766700 | 4.0825 | | 6.85531 | 7.534436 |
| | 19.49468 | 9.22105 | 5439000 | 289600 | 3.841 | 2.5045 | 8.851907 | 10.70157 |
| | 13.95731 | 6.919245 | 9004800 | 84100 | 2.405 | 3.4995 | 9.11639 | 8.213374 |
| | 24.19821 | 7.811357 | 12136000 | 345910 | 10.539 | 18.9705 | 7.650328 | 7.01773 |
| Average: | 18.4693 | 7.9273 | 9028450 | 306665 | 5.216875 | 6.5335 | 7.946913 | 8.108873 |
| SD: | 5.3777 | 1.0542 | 2608328 | 278957 | 3.62452269 | 8.346592958 | 0.992123319 | 1.526253977 |

### Materials and Methods

Study Design

The present disclosure investigates the capacity of an antibody targeting the catalytically active site of a prostate-specific protease (in man) to delineate and guide treatment of primary and metastatic prostate and breast cancer. Binding properties and cellular interaction were evaluated in vitro and in vivo using fluorescent and radio conjugates. The internalization of this antibody, via the neonatal Fc receptor, following interaction with its secreted targeted antigen, was studied in detail and evaluated in a second antibody targeting another secreted antigen. Appending the positron-emitting zirconium-89 to the antibody for immunoPET was studied in subcutaneous, osseous and hepatic metastatic, and genetically engineered autochthonous prostate cancer models. Tumor uptake and uptake kinetics were measured using manually defined regions of interest at multiple time points from 4 h through to 320 h. Imaging studies in bone and GEM systems were designed to measure treatment effect on AR-activity with surgical and/or chemical castration. Breast cancer cell lines were evaluated for KLK2 expression and hK2 production with and without hormone stimulation. To study BCa hk2 production in vivo, BT474 xenografts with and without androgen stimulation were imaged by ⁸⁹Zr-11B6. Quantitative in vivo PET imaging data was assessed in addition to ex vivo autoradiography and gamma counting. PET study duration was sufficiently long to achieve 20E6 coincident events, Cohorts in treatment groups were randomized and no outliers were excluded.

All chemicals and reagents of the highest available purity were purchased from ThermoFisher Scientific, unless otherwise noted. Murine 11B6 was provided by Dr. Kim Pettersson, University of Turku, Finland, while humanized 11B6 (hu11B6) was developed by DiaProst Inc., Lund, Sweden and produced by Innovagen Inc., Lund, Sweden. Enzalutamide (MDV3100), manufactured by Medivation, was provided Dr. Charles Sawyers at MSKCC.

Preparation of Zirconium-89

Zirconium-89 was produced through the ⁸⁹Y(p,n)⁸⁹Zr transmutation reaction on an EBCO TR19/9 variable-beam energy cyclotron (Ebco Industries, Inc.) in accordance with previously reported methods. ⁸⁹Zr-oxalate was isolated in high radionuclidic and radio-chemical purity >99.9 with an effective specific activity of 195 to 497 MBq/μg (5.27-13.31 mCi/μg). Immediately prior to radiolabeling, ⁸⁹Zr[Zr]oxalate was neutralized with aliquots of NaCO₃ (1 M) to pH 7.

Preparation of Radiolabeled Construct

Prior to conjugation, all antibodies were exchanged into 2-[4-(2-hydroxyethyl)piperazin-1-yl]ethanesulfonic acid (HEPES; 0.1 M, pH 8) by repeated ultracentrifugation (Amicon Centriplus YM-50, Millipore) and gel purification (PD10, GE Healthcare). The zirconium chelator, Deferoxamine-p-SCN (Areva Med) was conjugated to the antibody using a molar excess of 7:1. After addition of the bifunctional chelate the reaction was pH adjusted to pH 8.5 with Na₂CO₃ shaken at 37° C. for 1 hour, then purified by repeated centrifugation as above, into phosphate buffered saline (PBS). DFO-conjugated 11B6 (400 μL) was mixed with neutralized ⁸⁹Zr[Zr] and mixed gently. The pH after mixture was cross-checked and adjusted to pH 7, if needed. The labeling reaction was allowed to proceed for 1 hour. The conjugate was then purified by repeated purification by ultrafiltration into sterile saline. Radiochemical yield was assessed after purification average yield was between 40% and 50%. Radiopurity was assessed by radio-instant thin layer chromatography. Briefly, ⁸⁹Zr-DFO-11B6 (⁸⁹Zr-11B6) was blotted (1 μL) on silica-impregnated paper and eluted with a solution of 50 mM diethylenetriaminepentaacetic acid. All labeling reactions achieved >99% radiochemical purity. Average specific activity of the final radiolabeled conjugate was 1.4 mCi/mg.

Preparation of Fluorescently Labeled Constructs

Prior to conjugation, all antibodies were purified as above. The near infrared fluorophore Cy5.5-NHS (GE Healthcare) was resuspended in methanol aliquoted and dried using a speedvac. Using a molar excess of 3:1, the antibody was labeled and the pH was adjusted to 8.5 with Na₂CO₃. The reaction was shaken at 22° C. for 4 hours, followed by gel purification (PD10) and ultrafiltration (Amicon). The number of dye molecules per antibody was evaluated using a spectrophotometer and calculated to be 1.3 (SpectraMax M5, Molecular Devices). The dye labeled antibody conjugate was prepared fresh for each experiment.

Cell Lines

LNCaP, DU-145, CWR22Rv1, MDAPCa2b, VCaP were purchased from American Type Culture Collection. The cell lines were cultured according to the manufacturer's instructions. LAPC4, LREX′ and LNCaP-AR-luc was previously developed and reported by the Sawyers laboratory.

Animal Studies

All animal experiments were conducted in compliance with institutional guidelines at Memorial Sloan-Kettering Cancer Center. For xenograft studies: male athymic BALB/c (nu/nu) mice (6-8 weeks old, 20-25 g) were obtained from Charles River. LNCaP, DU-145, CWR22Rv1, MDAPCa2b, LAPC4, and VCaP tumors were inoculated in the right flank by subcutaneous injection of 1-5×10⁶ cells in a 200 μL cell suspension of a 1:1 v/v mixture of media with Matrigel (Collaborative Biomedical Products, Inc.). Tumors developed after 3 to 7 weeks. Enzalutamide (ENZ, MDV3100) was dissolved in dimethyl sulfoxide (DMSO) so that the final DMSO concentration when administered to animals would be 5. The formulation of the vehicle is 1 carboxymethyl cellulose, 0.1 polysorbate 80, and 5 DMSO. Enzalutamide or vehicle was administered daily by gavage. Liver xenografts of the LREX′ line were implanted.

Flank xenografts of the BT474 cell line were established using established procedures. Briefly, 17β-estradiol pellets (0.72 mg/pellet) (Innovative Research of America, Sarasota, Fla.) were inserted subcutaneously prior to inoculation of 1×10⁶ cells in a 200 μL suspension of a 1:1 v/v mixture of media with Matrigel (n=6). For this study, female Balb/c nu/nu animals were used. Animals in the DHT-positive group were supplemented with an additional subcutaneous 12.5 mg DHT pellet (Innovative Research of America).

Preparation of Osseous Tumor Grafts

Male CB-17 severe combined immunodeficient (SCID) mice (6-8 weeks old) were anesthetized with a mixture of ketamine/xylazine, and a parapatellar incision was made in the left hindlimb. The tibia was punctured using a needle, and 1×10⁵ cells (VCaP-luc or LNCaP-AR) were injected into the cavity. The puncture was closed with bone wax, the incision sutured, and animals received a palliative dose of carprofen (5 mg/kg) once daily for 3 days post inoculation. Tumor development was followed with bioluminescence imaging and confirmed with CT.

Biodistribution Studies

Biodistribution studies were conducted to evaluate the uptake of ⁸⁹Zr-11B6 in human prostate cancer xenograft models. Mice received ⁸⁹Zr-11B6 [3.7-5.55 MBq (100-150 μCi), 300, 100, 50, or 15 μg of protein, in 150 μL sterile saline for injection] through intravenous tail-vein injection (t=0 hour). Animals (n=4-5 per group) were euthanized by CO₂ asphyxiation at 24, 72, 96, 120, 240 and 344 hours post-injection and blood was immediately harvested by cardiac puncture. Eleven tissues (including the tumor) were removed, rinsed in water, dried on paper, weighed, and counted on a gamma-counter for accumulation of ⁸⁹Zr radioactivity. Count data were corrected for background activity and decay and the tissue uptake [measured in units of percentage injected activity per gram (% IA/g)] for each sample was calculated by normalization to the total amount of activity injected.

Small-Animal Positron Emission Tomography Imaging

PET imaging experiments were conducted on a micro-PET Focus 120 scanner (Concorde Microsystems). In initial studies, mice (n=4) were administered formulations of ⁸⁹Zr-11B6 [3.7-5.55 MBq (100-150 μCi), 300, 100, 50, or 25 μg of protein, in 150 μL sterile saline for injection] through i.v. Tail-vein injection. Approximately 5 minutes before recording PET images, mice were anesthetized by inhalation of 1% to 2% isoflurane (Baxter Healthcare)/oxygen gas mixture and placed on the scanner bed. PET images were recorded at various time points between 1 and 344 hours post-injection. List-mode data were acquired using a γ-ray energy window of 350 to 750 keV and a coincidence timing window of 6 nanoseconds. PET image data were corrected for detector non-uniformity, dead time, random coincidences and physical decay. For all static images, scan time was adjusted to ensure between 15-25 million coincident events were recorded.

Data were sorted into 3-dimensional histograms by Fourier rebinning, and transverse images were reconstructed using a maximum a priori algorithm to a 256×256×95 (0.72×0.72×1.3 mm) matrix. The reconstructed spatial resolution for ⁸⁹Zr was 1.9 mm full-width half-maximum at the center of the field of view. The image data were normalized to correct for non-uniformity of response of the PET, dead-time count losses, positron branching ratio, and physical decay to the time of injection, but no attenuation, scatter, or partial-volume averaging correction was applied. An empirically determined system calibration factor [in units of (mCi/mL)/(cps/voxel)] for mice was used to convert voxel count rates to activity concentrations. The resulting image data were then normalized to the administered activity to parameterize images in terms of percent injected activity per gram (% IA/g). Manually defined 3-dimensional regions of interest (also referred to as volumes of interest) were used to determine the maximum and mean % IA/g (decay corrected to the time of injection) in various tissues. Images were analyzed using ASIPro VM software (Concorde Microsystems).

Small-Animal CT Imaging and Co-Registration

Animals that were scanned on both PET and X-ray computed tomography (CT) systems were placed on a custom built platform in a rigid body fixed position (using 0.1 mm polyethylene wrapping). The bed was placed into an integrated heated-air, aneshthesia bed (MultiCell, Mediso). The bed was fixed in place on the microPET gantry and imaged as above. The bed was then moved for CT imaging using the NanoSPECT/CT (Bioscan). General acquisition parameters were 55 kVp with a pitch of 1 and 240 projections in a spiral scan mode. The entire animal was scanned using a multiple field of view procedure (with an approximate field of view of 4×4×4 cm per bed position), commonly requiring three bed positions per scan. Total scan time was approximately 10 min. A Shepp-Logan filter was used during the reconstruction process to produce image matrices with isotropic volumes of 221 μm.

PET data was reconstructed using a 3D filtered back projection maximum a priori algorithm using a ramp filter with a cut-off frequency equal to the Nyquist frequency into a 128×128×95 matrix. Data was exported in raw format and the rigid body (3 degrees of freedom) co-registration between PET and CT data (and MR, if applicable) was performed in Amira 5.3.3 (FEI). Amira and FIJI was used to produce the majority of the figures herein.

Fluorescent Microscopy/Surgical Imaging/Confocal Microscopy

Micrographs were acquired using an Eclipse Ti inverted microscope (Nikon) equipped with a motorized stage (Prior Scientific Instruments Ltd.), X-cite light source (EXFO) and filter sets (Chroma). Images were acquired and processed using NIS-Elements (Nikon), FIJI (NIH) and MosaicJ (Phillipe Thévenaz, Biomedical Imaging Group, Swiss Federal Institute of Technology Lausanne). All fluorescent images were captured with a fixed exposure time (fluorophore dependent).

Laser scanning confocal microscopy used the TCS SP8 (Leica) in the Molecular Cytology Core Facility (MCCF) of MSKCC. Cells were plated on glass bottom dishes (NUNC) for 48 hours, washed and then incubated for the noted time with Cy5.5-IgG (control), Cy5.5-11B6 and/or excess blocking 11B6 in supplemented media. Samples were scanned for Cy5.5.

Cellular Internalization Assay

VCaP, LNCaP and BT474 (with and without DHT stimulation) cells, cultured according to ATCC guidelines were incubated with ⁸⁹Zr-11B6 containing media. Uptake mechanism studies used purified human non-specific IgG (400 μg/1 mL/well, Invitrogen), human TruStain FcX Fc receptor blocking (40 μL/1 mL/well, Biolegend) or h11B6 (Fab′)₂ (0.2 mg/l mL/well, DiaProst Corp.) added together with the radioactive antibody. Control wells contained 20-fold excess of unlabeled antibody (to test specificity). Antibody concentrations were selected in preliminary experiments; a 20-fold increase in the antibody concentration did not significantly increase the amount of antibody bound. Triplicate samples were periodically removed, and cells were washed with 1 mL PBS (w/o Ca²⁺ and Mg²⁺+0.2% BSA). Lysate generated (1 mL of 1M NaOH for 5 min) was gamma counted. Cell uptake was determined by calculating percent activity found in cell lysate [100*(cell lysate activity/total activity)].

Confocal laser scanning microscopy was performed on cells beginning 12 h after incubation with 1:200 of either Cy5.5-11B6, phAb-11B6 (Promega Cat. No. G9841) or control Cy5.5-IgG. For FcRn co-localization, cells were fixed, permeabilized and stained using anti-FcRn Alexa-488 (Fisher, Cat. No. NBP189128-FCGRT).

Affinity Tests of ⁸⁹Zr-DFO-11B6, DFO-11B6 and H435A-11B6

Biotinylated 11B6 (100 μL; 2 mg/L) was added to streptavidin-coated microtiter plates, followed byl h of incubation with shaking. The plate was washed, after which 20, 100, 200, 400 or 1000 μg of compound (antibody) in 100 μL of DELFIA Assay Buffer was added to the wells, in duplicates, to compete with the capture antibody. Samples containing 0.34 ng/ml, or 3.4 ng/ml, in 100 μL of DELFIA Assay Buffer was hereafter added to the wells. After 2 h incubation with shaking, the plate was washed, and the Eu³⁺ labeled tracer antibody 6H10 was added (200 μL; 0.5 mg/L). The plate was incubated for 1 h with shaking, and then washed. DELFIA Enhancement Solution (200 μL) was added, and 5 min later, the time-resolved fluorescence was measured.

Time-Resolved Immunofluorometric Assay of Free and Total hK2

Total hK2 was measured using an in-house research assay that has previously been described by Vaisanen et al. Briefly, streptavidin coated micro-titer plates were incubated with biotinylated catcher antibody 6H10, followed by washing and incubation with samples and standards. After another round of washing, europium labeled tracer antibody 7G1 is added. After incubation and washing steps, enhancement solution is added prior to reading the plates. Free hK2 is measured in a similar fashion with biotin labeled 11B6 as a capture antibody and Europium labeled 6H10 as tracer antibody. Both assays have a functional detection limit of 0.04 ng/ml.

Tissue Lysate Preparation and Total Protein Measurement

Prostate tissues, harvested from transgenic mice were homogenized in lysis buffer (50 mM Sodium Acetate, 2 mM EDTA, 1% Triton X-100, lx complete protease inhibitor (Roche), and 10 mM benzamidine), sonicated for ten seconds (550 Sonic Dismembrator, Fisher Scientfic) and centrifuged at 13,000 rpm for 10 min. The supernatant was saved for analysis of determine free and total hK2 levels. Total protein levels were determined in homogenates using the BioRad DC Protein assay.

RNA Isolation and Quantitative-PCR

Approximately 200 mm³ of tumor sample was placed in a FastPrep Lysing Matrix tube (MP Biomedicals). Tumors were then homogenizing in 500 μL of Trizol (Ambion) using a FastPrep-24 instrument (MP Biomedicals). For xenograft tumors, the samples were transferred to a new eppendorf tube where 100 μg of glycogen (Ambion) was added. The samples were mixed by inversion and allowed to sit at room temperature for 5 min. Chloroform (100 μL; OmniSolv) was added and the samples were shaken vigorously and incubated for 3 min. The samples were then centrifuged at 11,500 rpm at 4° C. for 15 min and the aqueous (top) phase was transferred to a new eppendorf tube. Isopropanol (250 μL) of was added to the sample by pipetting until a precipitate formed. The sample was then centrifuged at 11,500 rpm at 4° C. for 10 min. The pellet was washed with 75-80% EtOH in DEPC water (Ambion). RNA was then purified using RNeasy Mini Kit (Qiagen) or the PureLink RNA Mini Kit (Ambion). RNA quality and quantity was determined using a spectrophotometer at 260 and 280 nm (Nanodrop-2000, Thermo Scientific). cDNA was generated using the High Capacity cDNA Reverse Transcription Kit (Applied Biosystems; Life Technologies). Quantitative-PCR was done using QuantiFast Sybr Green PCR Kit and RT² qPCR primers (Qiagen) on a RealPlex⁴ Mastercycler system (Eppendorf). KLK2 expression was quantified relative to beta actin using the comparative CT method.

Single Cell Extractions of Prostatic Tissue

A suspension of single cells was derived from the excised mouse prostatic tissue (of animals dosed with 100 μg of Cy5.5-11B6) following mastication at 4° C., digestion for 3 h in collagenase/hyaluronidase in culture media (DMEM with 5% FBS) at 37° C., incubation in trypsin for 1 h at 4° C. followed by low speed centrifugation. The cell pellet was resuspended in 5 mg/mL dispase and 1 mg/mL DNase I and pipetted gently, before being passed through a 70 μm strainer (ThermoFisher). All reagents were purchased from Stem Cell Technologies unless otherwise noted. Aliquots of the suspension were placed between two glass coverslips and scanned on the Eclipse Ti, as above.

Statistical Analysis

Data are presented as means±standard of the mean, unless otherwise noted. Statistical significance was analyzed by nonparametric student t test. Pearson's correlation coefficients were used for assessing the strength of association between pairs of predefined variables. In all cases, differences in results were considered to be statistically significant when the computed P value was less than 0.05. All tests were two-tailed. Analyses were performed using Prism 6.0 (Graphpad).

Expression and Purification of hu11B6

HEK293 cells were expanded to a cell density of 1×10⁶ cells/mL in a 2 L suspension culture in FreeStyle 293 Expression Medium (Life Technologies). The plasmid DNA (expression vectors p11B6VLhV1hk and p11B6VHhV1hIgG₁) containing the nucleotide sequences for the heavy and light chains of hu11B6 IgG1/k was then mixed with the transfection agent and incubated for 10 min at room temperature (RT). The DNA transfection agent mix was slowly added to the cell culture while slowly swirling the flask. The transfected cell culture was then incubated at 37° C. with 8% CO₂ on an orbital shaker platform rotating atabout 135 rpm for seven days. Culture medium was harvested by centrifugation and filtered through 5 μm, 0.6 μm, and 0.22 μm filter systems. Antibodies were purified by Protein G chromatography, and the buffer was changed to PBS pH 7.4 by dialysis; subsequently, the antibodies were concentrated by ultrafiltration. Concentration was measured by absorbance. Overall yield was 13.1 mg (˜6.5 mg/L).

Tissue Histology and Autoradiography

After mice were euthanized, a tissue package containing prostate lobes, seminal vesicles, and prostatic urethra was surgically excised and incubated in Tissue-Tek optimal cutting temperature compound (Sakura Finetek USA, Inc.) on ice for 45 minutes, and then snap-frozen on dry ice in a cryomold. Sets of contiguous 15 or 100 μm-thick tissue sections were cut with a CM1950 cryostat microtome (Leica Microsystems Inc.) and arrayed onto SuperfrostPlus glass microscope slides. Sections stained for actin and DNA (100 μm sections) were incubated with 200 μL of 10 U/mL rhodamine-phalloidin (Life Sciences Inc.) in PBS for 2-3 hours at RT in a covered container to prevent evaporation, and then washed with PBS twice. DNA/nuclei staining was performed by incubating the slides for 10 min in 5 μg/mL DAPI in PBS, followed by a wash with PBS. Slides were then air-dried, and a drop of Mowiol A-48 (Calbiochem Inc.) was placed on the slide before adding a mounting cover glass. Slides were then stored at −20° C. Immunostaining for AR was performed by incubating slides with blocking solution (2% BSA in PBS) for 15 min at room temperature and staining with 1:200 dilution of anti-AR polyclonal antibody (NH27) for 45 min followed by Texas red-conjugated goat anti-rabbit antibody (ICN) for 45 min at room temperature. Stained slides were then washed and mounted.

Sections intended for autoradiography were fixed in 4% paraformaldehyde solution in phosphate-buffered saline (Affymetrix) for 5 minutes, washed twice, air-dried, and stained with hematoxylin and eosin (H&E). The immunohistochemical detection of Ki-67, AR (N-20), and c-MYC was performed at the Molecular Cytology Core Facility of Memorial Sloan Kettering Cancer Center using a Discovery XT processor (Ventana Medical Systems). Before staining, all sections were blocked for 30 minutes in 10% normal goat serum with 2% BSA in PBS. Sections stained for Ki-67 were incubated with 0.4 μg/mL of the primary antibody (rabbit polyclonal Ki-67 antibody; Vector Labs, cat.#: VP-K451) for 2 hours, followed by a 30-minute incubation with biotinylated goat anti-rabbit IgG (Vector Labs, cat.#:PK6101) at 1:200 dilution. Sections stained for AR (N-20) were incubated for 3 hours with a polyclonal rabbit antibody (Santa Cruz, cat.#: SC-816) at 1 μg/ml concentration, followed by 16 minutes of incubation with biotinylated goat anti-rabbit IgG (Vector labs, cat#:PK6101) at 1:200 dilution. C-MYC staining was performed by incubating sections for 5 hours with a primary anti-c-MYC antibody (N terminal, rabbit polyclonal, Epitomics, cat.#: P01106), followed by 60 minutes of incubation with biotinylated goat anti-rabbit IgG (Vector Labs, cat.#: PK6101) at 1:200 dilution. Blocker D, streptavidin-HRP, and DAB detection kit (Ventana Medical Systems) were used according to the manufacturer's instructions. Stained tissue sections were placed in a film cassette against a Fuji film BAS-MS2325 imaging plate (Fuji Photo Film Co.) to acquire digital autoradiograms. The slides were exposed for 48 hours, approximately 168 hours after injection of ⁸⁹Zr-DFO-11B6. Exposed phosphor plates were read by a Fujifilm BAS-180011 bio-imaging analyzer (Fuji Photo Film Co.), generating digital images with 50 μm pixel resolution. Digital images were obtained with an Olympus BX60 System Microscope (Olympus America, Inc.) equipped with a motorized stage (Prior Scientific, Inc.). Subsequently, H&E images were acquired to the same resolution as the DAR data. DAR images were manually aligned to the H&E images using rigid planar transforms.

Transgenic KLK2 Mouse Models

Site-directed mutagenesis of APLILSR to APLRTKR at positions 4, -3, and -2 the zymogen sequence of KLK2 was performed using a Quick Change Lightning Mutagenesis Kit (Stratagene). This enabled furin, a ubiquitously expressed protease in rodent prostate tissue, to efficiently cleave the short activation peptide at the cleavage site (−1 Arg/+1 Ile), resulting in functional hK2. Sequencing was performed to verify the genotype using the following primers: 5′-TTC TCT AGG CGC CGG AAT TA-3′ (forward), 3′-CCC GGT AGA ATT CGT TAA CCT-3′ (reverse). A transgenic mouse model was established by cloning the described construct into a SV40 T-antigen cassette downstream of the short rat probasin promoter (pb). This construct was microinjected into fertilized mouse embryos (C57BL/6) and implanted into pseudopregnant female mice. A cancer-susceptible transgenic mouse model with prostate specific hK2 expression was created by crossing the pb_KLK2 transgenic model with the Hi-MYC model (ARR2PB-Flag-MYC-PAI transgene). A schematic of the strategies used is included as FIG. 31. Integration of genes into the genome of the offspring was confirmed by Southern blot analysis and PCR. Mice were monitored closely in accordance with IACUC-established guidelines and RARC animal protocol (#04-01-002).

Castration- and Enzalutamide-Resistant Liver Metastasis Model

Previously surgically castrated mice with a body weight of 28-30 g were anesthetized by intraperitoneal injection of ketamine (75 mg/kg) and xylazine 2% (15 mg/kg). Anesthetized animals were placed in a supine position, draped, and prepared for sterile surgery. A 10 mm midline incision was made on the upper abdomen through the skin and peritoneum. The left lobe of the liver was separated from the caudate and median lobe, and was exposed and immobilized. A Hamilton syringe with a 26-gauge needle was used for injection of a 10 mixture of LREX′ tumor cells (10⁵ cells) and Matrigel (1:1). The puncture site was closed by gentle pressure for approximately 1 min with a moistened cotton-tipped applicator stick. After tumor cell inoculation, the liver lobe was repositioned anatomically. The abdominal wall was then closed in a two-layer technique with a resorbable suture for the fascia and subcutaneous tissue (5/0 vicryl, Ethicon) and a nonresorbable suture for the skin (5/0 prolene, Ethicon). A 0.05 mg dexamethasone pellet (60 day release) was subcutaneously implanted at the end of the procedure to confer enzalutamide resistance and activate the glutocorticoid receptor (47). Animals received postoperative analgesia by subcutaneous injection of carprofen (5 mg/kg) once daily for 3 days after surgery. Daily enzalutamide (10 mg/kg) treatment was given by gavage. Tumor development was followed with bioluminescence imaging and confirmed with MR imaging.

Antibody Humanization

The acceptor framework used for the grafting was derived from the human immunoglobulin germline genes showing the highest sequence similarity with the variable domains of the parental 11B6 antibody. The genes were identified by comparing the amino acid sequences of the mouse 11B6 variable light (V_{L}) and heavy (V_{H}) domains to the human immunoglobulin germline sequences in NCBI database. The germline V gene IGKV4-1*01 (GenBank: Z00023.1) together with the short IGKJ2 gene (GenBank: J00242.1) were selected to construct the V_{L} acceptor framework into which the CDRs of mouse 11B6 light chain were grafted. For the V_{H} acceptor framework, the V gene IGHV4-28*01 (GenBank: X05714.1) and J gene IGHJ1 (GenBank: AAB59411.1) were used. A 3D homology model of the mouse 11B6 was built to facilitate the evaluation of the influence of non-CDR residues on the CDR loop conformations. On the basis of the published data and visual inspection of the model, the following residues were adopted from the parental mouse 11B6: Leu4 in the light chain and Asn27, Thr30, Arg71, and Thr94 in the heavy chain. On the basis of structural analysis, certain CDR residues were obtained from the sequences of the human acceptor framework: an arginine was introduced in the position 54 in CDR-L2 to allow the formation of a salt bridge with another light chain residue Asp60, whereas Lys24 in CDR-L1 and Asn60 in CDR-H2 were included to maximize the content of human gene-derived amino acids in hu11B6, although they were predicted not to play a major role in antigen binding.

Codon optimized nucleotide sequences encoding hu11B6 variable heavy or light chains were designed, purchased as synthetic genes, and subcloned to obtain the mammalian expression vectors p11B6VLhV1hk (4300 bp) and p11B6VHhV1hIgG₁ (4900 bp) for the production human IgG₁/kappa antibody.

11B6 Immunohistochemistry

The murine 11B6 antibody was used on human tissue microarrays. Human tissue microarrays (US Biomax) included fine needle biopsies of normal prostate, primary adenocarcinoma, and metastatic foci. Four-μm sections were deparrafinized in xylene and rehydrated in decreasing ethanol dilutions. Endogenous peroxidase was blocked with 3% hydrogen peroxide buffer for 10 minutes. Antigen retrieval was performed by boiling in EDTA buffer (pH 9.0) for 20 min. Slides were subsequently incubated overnight in a humidified chamber with murine anti-hK2 (m11B6) at a 1:1000 dilution in 0.5% BSA/TBST followed by one hour incubation with Poly-HRP-anti-mouse/rabbit/rat IgG (Brightvision, Immunologic). The slides were developed with diaminobenzidine and lightly counterstained with hematoxylin and mounted.

FcRn Affinity Measurements

To test the effect of the H435A-11B6 antibody, which contains a point mutation (and the original 11B6 construct), surface plasmon resonance (SPR) was performed on a CMS chip using a Biacore 3000 instrument. The chip and all reagents were purchased from GE Healthcare; experiments were conducted in assay buffer (67 mM phosphate buffer, 0.15 M NaCl, 0.05% Tween-20) adjusted to either pH 6.0 or pH 7.4. At the lower pH, FcRn has the ability to bind to the Fc portion of intact immunoglobulins (IgG₁), but at the higher pH this affinity drops to enable release of the antibody (24). Human FcRn (hFcRn) was bound to the chip by following the manufacturer's guidelines, with carbodiimide (EDC) and N-hydroxysuccinimide (NETS) in reaction buffer (10 mM sodium acetate, pH 5.0) and washed after immobilization with running buffer. Channels were blocked by ethanolamine after activation and immobilization and EDC and NETS washed off. The affinity of each antibody for the FcRn was evaluated with a flow rate of 30 μL/min at a concentration of 50 nM in each buffer condition. If binding was observed, association and dissociation rates were measured using the bivalent fitting model (BIAevaluation Software, Biacore).

Characterization of h11B6 Affinity

After optimizing the experimental conditions, multiple binding measurements were performed for m11B6, hu11B6, DFO-hu11B6, and the antigen. From the collected data, the association and dissociation rate constants (kₒₙ and k_{off}) and the dissociation constants (K_{D}) were calculated.

### EQUIVALENTS

While systems, methods, and compositions have been particularly shown and described with reference to specific preferred embodiments, it should be understood by those skilled in the art that various changes in form and detail may be made therein without departing from the spirit and scope of the invention as defined by the appended claims.

What is claimed is:

1. A method of assessing androgen receptor activity in a subject, the method comprising:
administering, to the subject, a tracer-labelled hK2-specific or PSA-specific antibody; and
detecting the presence of the labeled antibody in a tissue of the subject.

2. The method of claim 1, wherein the tissue comprises breast tissue.

3. The method of claim 1 or 2, wherein the antibody comprises a murine or humanized antibody.

4. The method of any one of claims 1 to 3, wherein the antibody comprises murine or humanized 11B6, and/or murine or humanized 5A10.

5. The method of any one of the preceding claims, wherein the tracer comprises a radionuclide.

6. The method of claim 5, wherein the radionuclide is a member selected from the group consisting of ¹¹C, ⁶⁴Cu, ¹²⁴I, ¹¹¹In, ¹⁷⁷Lu, ¹⁵O, ¹⁸F, ⁶⁸Ga, ⁸⁹Zr, and ⁸²Rb.

7. The method of any one of the preceding claims, comprising administering hu11B6 labeled with ⁸⁹Zr or administering ⁸⁹Zr-DFO-hu11B6.

8. The method of any one of the preceding claims, wherein detecting is performed via PET imaging, CT imaging, SPECT imaging, and/or in vivo imaging.

9. The method of claim 8, comprising detecting the presence and/or activity of the androgen receptor (AR) axis.

10. The method of any one of the preceding claims, comprising detecting the presence of the labeled antibody in the tissue at a time frame selected from the group consisting of at least 24 hours after administration of the labeled antibody to the subject, at least 48 hours after administration of the labeled antibody to the subject, at least 100 hours after administration of the labeled antibody to the subject, and at least 120 hours after administration of the labeled antibody to the subject.

11. The method of claim 10, wherein the labeled antibody accumulates and internalizes in tumor cells, thereby allowing visualization/tracking over long periods of time.

12. The method of any one of the preceding claims, wherein the tissue has metastasized to bone.

13. The method of any one of the preceding claims, comprising detecting the presence of the labeled antibody in the tissue over a period of multiple time intervals.

14. The method of claim 13, wherein the detecting is for real-time monitoring/visualization.

15. The method of claim 13 or 14, comprising detecting the presence of the labeled antibody in the tissue at multiple times, including at least one detection after a time selected from the group consisting of at least 24 hours following administration of the labeled antibody, after at least 48 hours following administration of the labeled antibody, after at least 100 hours following administration of the labeled antibody, and after at least 120 hours following administration of the labeled antibody.

16. The method of any one of the preceding claims, further comprising one or more of (i) to (vi), as follows:
(i) identifying the presence of cancer in the subject;
(ii) localizing a cancer in the subject;
(iii) quantitatively assessing androgen receptor pathway activity in the subject/cancer;
(iv) planning radiation dose(s) in a course of treatment of the subject;
(v) determining one or more pharmacodynamics parameters for the subject; and
(vi) evaluating treatment efficacy.

17. The method of claim 16, wherein the cancer comprises a member selected from the group consisting of breast cancer (BCa), AR-positive breast cancer, triple negative breast cancer (TN-BCa), and any metastasis of BCa. Ar-positive breast cancer, and TN-BCa.

18. The method of claim 16 or 17, wherein the determining of one or more pharmacodynamics parameters for the subject is in conjunction with treatment of the subject with one or more drugs.

19. The method of any one of claims 16 to 18, wherein the evaluating comprises evaluating therapy response.

20. The method of any one of claims 16 to 19, comprising monitoring AR-upregulation of KLK2 and/or KLK3.

21. The method of claim 20, wherein the AR-upregulation of KLK2 and/or KLK3 is in response to external irradiation.

22. A method of assessing androgen receptor activity in a subject, the method comprising:
administering, to the subject, a tracer-labelled hK2-specific or PSA-specific antibody; and
detecting the presence of the labeled 11B6 in a tissue of the subject.

23. The method of claim 22, wherein the tissue comprises breast tissue.

24. The method of claim 22 or 23, wherein the tracer-labelled hK2-specific or PSA-specific antibody comprises a murine or humanized antibody.

25. The method of claim 24, wherein the murine or humanized antibody comprises a murine or humanized 11B6 (hu11B6), and/or murine or humanized 5A10 (hu5A10).

26. The method of any one of claims 22 to 25, wherein the tracer comprises a fluorophore.

27. The method of claim 26, comprising administering hu11B6 labeled with a tag comprising a member selected from the group consisting of a near infrared fluorophore and a Cy5.5.

28. The method of any one of claims 22 to 27, wherein the detecting is performed via fluorescent imaging or in vivo imaging.

29. The method of any one of claims 22 to 28, comprising detecting the presence and/or activity of the androgen receptor (AR) axis.

30. The method of any one of claims 22 to 29, further comprising one or more of (i) to (vi), as follows:
(i) identifying the presence of cancer in the subject;
(ii) localizing the cancer in the subject;
(iii) quantitatively assessing androgen receptor pathway activity in the subject/cancer;
(iv) planning radiation dose(s) in a course of treatment of the subject;
(v) determining one or more pharmacodynamics parameters for the subject; and
(vi) evaluating treatment efficacy.

31. The method of claim 30, wherein the cancer comprises a member selected from the group consisting of breast cancer (BCa), AR-positive breast cancer, triple negative breast cancer (TN-BCa), and any metastasis of BCa, AR-positive breast cancer, and TN-BCa.

32. The method of claim 30 or 31, wherein the determining of one or more pharmacodynamics parameters for the subject is determined in conjunction with treatment of the subject with one or more drugs.

33. The method of any one of claims 30 to 32, comprising monitoring AR-upregulation of KLK2 and/or KLK3.

34. The method of claim 33, wherein the AR-upregulation of KLK2 and/or KLK3 is in response to external irradiation.

35. A method of treating AR-positive breast cancer with one or more agents/treatments selected from the group consisting of:
(i) a radionuclide-labelled hK2-specific or PSA-specific antibody; and
(ii) at least one member selected from the group consisting of progesterone, testosterone, and external irradiation,
which method comprises administering the one or more agents/treatments to a subject suffering from or susceptible to AR-positive breast cancer, so that the subject is receiving therapy with a combination of (i) and (ii) above.

36. The method of claim 35, wherein the radionuclide comprises a member selected from the group consisting of ⁹⁰Y, ¹³¹I, ²¹¹At, ¹¹¹In, ¹⁷⁷Lu, ²²⁷Th, ¹⁴⁹Tb, ²¹²Bi, ²¹³Bi, ²²⁵Ac, ⁸²Rb, and ²²³Ra.

37. The method of claim 35 or 36, wherein the radionuclide-labelled hK2-specific or PSA-specific antibody comprises a member selected from the group consisting of a humanized 11B6 (hu11B6), humanized 5A10 (hu5A10), hu11B6 labeled with an alpha-particle-emitting radionuclide, hu11B6 labeled with ²²⁵Ac, and ²²⁵Ac-DOTA-hu11B6.

38. A method of treating AR-positive breast cancer or any metastasis of AR-positive breast cancer, the method comprising administering, to a subject suffering from or susceptible to the disease or condition, a radionuclide-labelled hK2-specific or PSA-specific antibody.

39. The method of claim 38, wherein the radionuclide comprises a member selected from the group consisting of ⁹⁰Y, ¹³¹I, ²¹¹At, ¹⁴⁹Tb, ²¹²Bi, ²¹³Bi, ²²⁵Ac, ¹¹¹In, ¹⁷⁷Lu, ²²⁷Th, and ²²³Ra.

40. A composition comprising one or more agents selected from the group consisting of:
(i) a radionuclide-labelled hK2-specific or PSA-specific antibody; and
(ii) at least one member selected from the group consisting of progesterone, testosterone, and external irradiation,
for use in a method of treating AR-positive breast cancer in a subject suffering from or susceptible to AR-positive breast cancer,
wherein the treating comprises:
delivering a combination of (i) and (ii) above to the subject.

41. A composition comprising one or more agents selected from the group consisting of:
(i) a radionuclide-labelled hK2-specific or PSA-specific antibody; and
(ii) at least one member selected from the group consisting of progesterone, testosterone, and external irradiation,
for use in therapy.

42. A composition comprising one or more agents selected from the group consisting of:
(i) a radionuclide-labelled hK2-specific or PSA-specific antibody; and
(ii) at least one member selected from the group consisting of progesterone, testosterone, and external irradiation,
for use in a method of in vivo diagnosis of AR-positive breast cancer in a subject in a subject suffering from or susceptible to AR-positive breast cancer,
wherein the in vivo diagnosis comprises:
delivering a combination of (i) and (ii) above to the subject.

43. A composition comprising one or more agents selected from the group consisting of:
(i) a radionuclide-labelled hK2-specific or PSA-specific antibody; and
(ii) at least one member selected from the group consisting of progesterone, testosterone, and external irradiation,
for use in in vivo diagnosis.

44. A composition comprising one or more agents selected from the group consisting of:
(i) a radionuclide-labelled hK2-specific or PSA-specific antibody; and
(ii) at least one member selected from the group consisting of progesterone, testosterone, and external irradiation,
for use in
(a) a method of treating AR-positive breast cancer in a subject or
(b) a method of in vivo diagnosis of AR-positive breast cancer in a subject,
wherein the method comprises:
delivering a combination of (i) and (ii) above to the subject.

45. The composition of any one of claims 40 to 44, wherein the radionuclide comprises a member selected from the group consisting of ⁹⁰Y, ¹³¹I, ²¹¹At, ¹¹¹In, ¹⁷⁷Lu, ²²⁷Th, ¹⁴⁹Tb, ²¹²Bi, ²¹³Bi, ²²⁵Ac, ⁸²Rb, and ²²³Ra.

46. The composition of any one of claims 40 to 45, wherein the radionuclide-labelled hK2-specific or PSA-specific antibody comprises a member selected from the group consisting of a humanized 11B6 (hu11B6), humanized 5A10 (hu5A10), hu11B6 labeled with an alpha-particle-emitting radionuclide, hu11B6 labeled with ²²⁵Ac, and ²²⁵Ac-DOTA-hu11B6.

47. A composition comprising a radionuclide-labelled hK2-specific of PSA-specific antibody for use in a method of treating AR-positive breast cancer or any metastasis of AR-positive breast cancer in a subject suffering from or susceptible to the disease or condition, wherein the treating comprises delivering the composition to the subject.

48. A composition comprising a radionuclide-labelled hK2-specific of PSA-specific antibody for use in therapy.

49. A composition comprising a radionuclide-labelled hK2-specific of PSA-specific antibody for use in a method of in vivo diagnosis of AR-positive breast cancer or any metastasis of AR-positive breast cancer in a subject suffering from or susceptible to the disease or condition, wherein the in vivo diagnosis comprises delivering the composition to the subject.

50. A composition comprising a radionuclide-labelled hK2-specific of PSA-specific antibody for use in in vivo diagnosis.

51. A composition comprising a radionuclide-labelled hK2-specific of PSA-specific antibody for use in
(a) a method of treating AR-positive breast cancer or any metastasis of AR-positive breast cancer in a subject or
(b) a method of in vivo diagnosis of AR-positive breast cancer or any metastasis of AR-positive breast cancer in a subject,
wherein the method comprises delivering the composition to the subject.

52. The composition of claim 47 or 51, wherein the radionuclide comprises a member selected from the group consisting of ⁹⁰Y, ¹³¹I, ²¹¹At, ¹⁴⁹Tb, ²¹²Bi, ²¹³Bi, ²²⁵Ac, ¹¹¹In, ¹⁷⁷Lu, ²²³Ra, and ²²⁷Th.
